(19) United States
(12) Patent Application Publication
(10) Pub. No.: US 2009/0099169 A1
(43) Pub. Date: Apr. 16, 2009

(54) Benzofuran Compounds As EP1 Receptor Antagonists

(75) Inventors: Mairi Gibson, Essex (GB); Adrian Hall, Essex (GB); David Nigel Hurst, Essex (GB); Derek Anthony Rawlings, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXO GROUP LIMITED, Greenford, Middlesex (GB)

(21) Appl. No.: 12/295,379

(22) PCT Filed: Apr. 3, 2007
(86) PCT No.: PCT/EP2007/053206

§ 371 Date: Sep. 30, 2008

(30) Foreign Application Priority Data
Apr. 5, 2006 (GB) 0606876.1

(51) Int. Cl. A61K 31/5377; C07D 407/06; A61K 31/341; C07D 405/12; A61K 31/4155; C07D 405/14; A61K 31/4523; C07D 413/12

(52) U.S. Cl. 514/233.5; 549/467; 514/469; 546/196; 514/326; 544/153; 548/364.4; 514/406

(57) ABSTRACT

Compounds of formula (I) or a pharmaceutically acceptable derivative thereof:

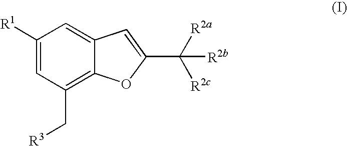

wherein R¹, R^{2a}, R^{2b}, R^{2c}, and R³ are as defined in the specification, a process for the preparation of such compounds, pharmaceutical compositions comprising such compounds and the use of such compounds in medicine.

This invention relates to benzofuran compounds, to processes for their preparation, to pharmaceutical compositions containing them and to their use in medicine, in particular their use in the treatment of conditions mediated by the action of PGE₂ at the EP₁ receptor.

Prostaglandin receptors, including the EP₁₋₄, DP, FP IP and TP receptors are the effector proteins for the products (prostaglandins) downstream of COX-1/2 activation (PGE₂, PGD2, PGF2a, PGI2 and thromboxane respectively). The NSAIDS (non-steroidal anti-inflammatory drugs) are indiscriminate cyclooxygenase inhibitors and reduce the levels of these prostaglandins. This in turn reduces the action of the prostaglandins at their respective receptors. In view of the relatively large number of receptors affected, the pharmacology of the NSAIDS is complex.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion.

We have now found a novel group of compounds which bind with high affinity to the EP₁ receptor. These compounds are antagonists of the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S, Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. In The American Physiological Society (1994, 267, R289-R-294), studies suggest that PGE₂-induced hyperthermia in the rat is mediated predominantly through the EP₁ receptor.

The TP (also known as TxA₂) receptor is a prostanoid receptor subtype stimulated by the endogenous mediator thromboxane. Activation of this receptor results in various physiological actions primarily incurred by its platelet aggregatory and smooth muscle constricting effects, thus opposing those of prostacyclin receptor activation.

TP receptors have been identified in human kidneys (G. P. Brown et al, Prostaglandins and other lipid mediators, 1999, 57 179-188) in the glomerulus and extraglomerular vascular tissue. Activation of TP receptors constricts glomerular capillaries and suppresses glomerular filtration rates (M. D. Breyer et al, Current Opinion in Nephrology and Hypertension, 2000, 9, 23-29), indicating that TP receptor antagonists could be useful for renal dysfunction in glomerulonephritis, diabetes mellitus and sepsis.

Activation of TP receptors induces bronchoconstriction, increase in microvascular permeability, formation of mucosal oedema and mucus secretion, typical characteristic features of bronchial asthma (T. Obata et al, Clinical Review of Allergy, 1994, 12(1), 79-93). TP antagonists have been investigated as potential asthma treatments resulting in, for example, orally active Seratrodast (AA-2414) (S. Terao et al, Yakugaku Zasshi, 1999, 119(5), 377-390). Ramatroban is another TP receptor antagonist currently undergoing phase III clinical trials as an anti-asthmatic compound.

Antagonists at the TP receptor have been shown to have a gastroprotective effect. In rats it has been shown that SQ 33961 and BM 13505 inhibit gastric lesions induced by taurocholate acid, aspirin or indomethacin (E. H. Ogletree et al, Journal of Pharmacology and Experimental Therapeutics, 1992, 263(1), 374-380.

Certain compounds of the present invention also exhibit antagonism at the TP receptor and are therefore indicated to be useful in treating conditions mediated by the action of thromboxane at the TP receptor. Such conditions include those disclosed in WO 2004/039807 (Merck Frosst Canada & Co) which is incorporated herein by reference, and include respiratory diseases e.g. asthma, allergic diseases, male erectile dysfunction, thrombosis, renal disorders and gastric lesions.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005), WO2005/108369 (17 Nov. 2005), WO 2006/066968 (29 Jun. 2006), WO 2006/114272 (2 Nov. 2006), WO 2006/114274 (2 Nov. 2006) and WO 2006/114313 (2 Nov. 2006) disclose compounds as being useful in the treatment of prostaglandin mediated diseases.

P. Lacombe et al (220th National Meeting of The American Chemical Society, Washington D.C., USA, 20-24 August, 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} August 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

DT 2602340 A1 discloses certain benzyl picolinic acid derivatives as hypotensive agents and dopamine β-hydroxylase inhibitors.

EP514217 (19 Nov. 1992), EP514198 (19 Nov. 1992), EP514193 (19 Nov. 1992), EP514192 (19 Nov. 1992), EP505954 (30 Sep. 1992), WO9209600 (11 Jun. 1992) and EP434249 (26 Jun. 1992) disclose benzofuran derivatives as angiotensin antagonists.

Accordingly the present invention provides one or more chemical entities selected from compounds of formula (I):

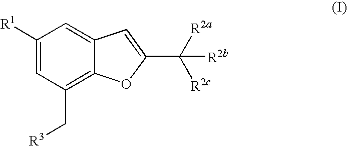

wherein
R¹ is hydrogen, halogen, CF₃, CN, SO₂CH₃ or CH₃;
R^{2a} is hydrogen, C₁₋₄ alkyl, or CF₃;
R^{2b} is C₁₋₄ alkyl or CF₃; and
R^{2c} is hydrogen or CH₃;
or R^{2a} and R^{2b} together with the carbon atom to which they are attached form a C₃₋₆ cycloalkyl group, and R^{2c} is hydrogen;

### R³ is:

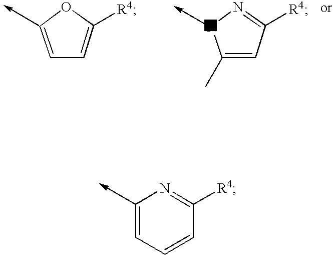

R⁴ is CO₂H, NHCO₂R⁵, CONR^{6a}R^{6b}, CONHSO₂R⁷, NHCONR⁸R⁹, NHCOR¹⁰, imidazole or tetrazole;
or R⁴ is an imidazole ring fused to give an optionally substituted bicyclic or tricyclic ring system;
R⁵ is C₂₋₆ alkyl, optionally substituted CH₂-phenyl or optionally substituted CH₂aliphatic heterocycle;
R^{6a} is hydrogen; and
R^{6b} is hydrogen; NR¹¹R¹²; C₁₋₆alkyl optionally substituted by NR¹¹R¹²; phenyl optionally substituted by halogen, CH₂OH, CH₂NR¹¹R¹², or optionally substituted CH₂aliphatic heterocycle; or optionally substituted (CH₂)ₙaliphatic heterocycle wherein n is 0, 1 or 2;
or R^{6a} and R^{6b} together with the nitrogen atom to which they are attached form an optionally substituted aliphatic heterocycle;
R⁷ is C₁₋₄alkyl, phenyl or heteroaryl;
R⁸ is hydrogen or C₁₋₄alkyl;
R⁹ is C₁₋₄alkyl;
R¹⁰ is C₃₋₆cycloalkyl optionally substituted by OH; C₁₋₆alkyl; or optionally substituted (CH₂)ₘaliphatic heterocycle wherein m is 0, or 1; or

### R¹⁰ is

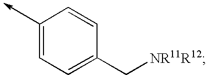

R¹¹ is hydrogen or C₁₋₄alkyl; and
R¹² is hydrogen or C₁₋₄alkyl;
or derivatives thereof;
provided that:
when R^{2c} is CH₃, then R⁴ is CO₂H; and
when R¹ is SO₂CH₃, then R⁴ is CO₂H.

In one aspect R¹ is hydrogen, Cl, Br, SO₂CH₃ or CN. In another aspect R¹ is hydrogen, Cl, Br, or CN. In a further aspect R¹ is Cl.

In one aspect R^{2c} is hydrogen.

In one aspect R^{2a}, R^{2b} and R^{2c} together with the carbon atom to which they are attached form a group selected from propyl, iso-propyl, tert-butyl, cyclopropyl or cyclohexyl. In another aspect R^{2a}, R^{2b} and R^{2c} together with the carbon to which they are attached form a group selected from isopropyl or C₃₋₆cycloalkyl.

In one aspect R³ is

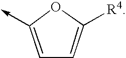

In another aspect R³ is

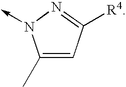

In a further aspect R³ is

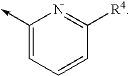

In one aspect R⁴ is CO₂H, NHCO₂R⁵, CONR^{6a}R^{6b}, or NHCOR¹⁰;

or R⁴ is an imidazole ring fused to give an optionally substituted bicyclic or tricyclic ring system. In another aspect R⁴ is CO₂H, NHCO₂R⁵, CONR^{6a}R^{6b}, NHCOR¹⁰ or optionally substituted benzimidazole. In a further aspect R⁴ is CO₂H.

When R⁴ forms a fused imidazole ring group, such groups include benzimidazole optionally substituted by CH₂OH, or CH₂NR^{x}R^{y} wherein R^{x} and R^{y} are independently selected from hydrogen and optionally substituted C₁₋₄alkyl, or R^{x} and R^{y} together with the nitrogen atom to which they are attached form an aliphatic heterocyclyl ring optionally containing another heteroatom selected from O, NH, NC₁₋₄alkyl, or S. In one aspect R^{x} and R^{y} are independently selected from hydrogen, CH₃ and C₂H₅; or R^{x} and R^{y} together with the nitrogen atom to which they are attached form an aliphatic heterocycle. When R^{x} and R^{y} together with the nitrogen atom to which they are attached form an aliphatic heterocycle, heterocycles include pyrrolidine or piperazine optionally substituted by methyl or ethyl. Suitably CH₂NR^{x}R^{y} is CH₂NHCH₃, CH₂N(CH₃)₂, CH₂pyrrolidine, or CH₂piperazinylmethyl. When R⁴ is substituted benzimidazole, preferably the substituent is on the benzimidazole 5-position.

In one aspect R⁵ is C₂₋₆ alkyl or CH₂aliphatic heterocycle. In another aspect R⁵ is tert-butyl or CH₂piperidin-4-yl.

In one aspect R^{6b} is hydrogen; NR¹¹R¹²; C₁₋₄alkyl optionally substituted by NR¹¹R¹²; phenyl optionally substituted by halogen, CH₂OH, CH₂NR¹¹R¹², or optionally substituted CH₂aliphatic heterocycle; or R^{6b} is optionally substituted (CH₂)ₙaliphatic heterocycle wherein n is 0, 1 or 2. When R^{6b} is optionally substituted (CH₂)ₙaliphatic heterocycle wherein n is 0, 1 or 2, in one aspect the aliphatic heterocycle is linked via a nitrogen atom. In another aspect, when R^{6b} is optionally substituted (CH₂)ₙaliphatic heterocycle wherein n is 0, 1 or 2, the aliphatic heterocycle is linked via a carbon atom.

In another aspect R^{6b} is hydrogen; dimethylamino; isopropyl; C₁₋₆alkyl optionally substituted by NR¹¹R¹² (e.g. CH₂CH₂N(CH₃)₂, CH₂C(CH₃)₂NH₂); phenyl optionally substituted by one or two substituents selected from fluorine, CH₂OH, CH₂N(CH₃)₂; and CH₂N(C₂H₅)₂; tetrahydropyran; piperidine optionally substituted by CH₃ or C₂H₅; CH₂pyrrolidine optionally substituted by OH; CH₂piperidine optionally substituted by OH, OCH₃ or OC₂H₅; CH₂morpholine; CH₂tetrahydropyran, or CH₂piperidine optionally substituted by CH₃ or C₂H₅. When R^{6b} is phenyl substituted by CH₂NR¹¹R¹² (e.g. CH₂N(CH₃)₂ or CH₂N(C₂H₅)₂) then the substituent is preferably attached to the para-position of the phenyl ring.

In one aspect R¹⁰ is cyclohexyl optionally substituted by OH; isopropyl; tetrahydropyran or tetrahydrofuran; or

### R¹⁰ is

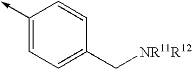

wherein CH₂NR¹¹R¹² is CH₂N(C₂H₅)₂ or CH₂pyrrolidine.

In one aspect the present invention provides one or more chemical entities selected from compounds of formula (Ia):

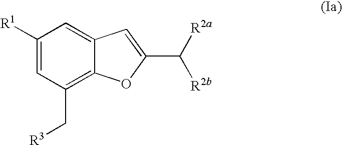

wherein
R¹ represents hydrogen, halogen, —CF₃ or methyl;
R^{2a} and R^{2b} independently represent C₁₋₄ alkyl, —CF₃ or R^{2a} and R^{2b} together with the carbon atom to which they are attached form a C₃₋₅ cycloalkyl group;
R³ represents

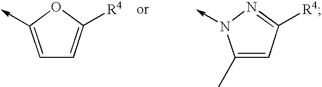

R⁴ represents —COOH, —NH—COO—R⁵, —CO—NH—R⁶, —CO—NH—SO₂—R⁷, tetrazole or R⁴ is an imidazole ring fused to give an optionally substituted bicyclic or tricyclic ring system;
R⁵ represents optionally substituted aliphatic heterocyclylalkyl;
R⁶ represents an aryl, aliphatic heterocyclyl or an -aryl-CH₂-aliphatic heterocyclyl group each of which may be optionally substituted;
R⁷ represents C₁₋₆alkyl, aryl or heteroaryl;
or derivatives thereof.

Optional substituents for aryl, aliphatic heterocyclyl or aliphatic heterocyclylalkyl moieties as a group or part of a group are selected from optionally substituted C₁₋₆alkyl, optionally substituted C₁₋₆alkylamino, hydroxy, optionally substituted C₁₋₆alkoxy and halogen.

Examples of fused imidazole groups include benzimidazole, imidazo[1,2-a]pyridine, imidazo[1,2-a]pyrazine, imidazo[1,2-a]pyrimidine, imidazo[4,5-b]pyridine, imidazo[4,5-b]pyrazine, imidazo[4,5-c]pyridine and purine, all of which may be optionally substituted. Suitable optional substituents include one or two substituents selected from halogen, e.g. Cl or F; 4-C₁₋₄alkylpiperazinyl e.g. 4-methylpiperazin-1-yl; OC₁₋₄alkyl, e.g. OCH₃; HOC₁₋₄alkyl, e.g. HOCH₂; CH₂NHC₁₋₄alkyl; CH₂N(C₁₋₄alkyl)₂; CH₂heterocyclyl, e.g. CH₂pyrrolidine, CH₂morpholine, CH₂C₁₋₄alkylpiperidine.

Suitably R¹ represents halogen (e.g. chlorine).

In one embodiment, R^{2a} and R^{2b} independently represent C₁₋₄ alkyl (e.g. methyl). In a further embodiment, R^{2a} and R^{2b} both represent methyl.

Suitably R⁴ represents optionally substituted-benzimidazolyl (e.g. -benzimidazolyl optionally substituted by an HOC₁₋₄alkyl (e.g. HOCH₂), CH₂NHC₁₋₄alkyl (e.g. —CH₂—N(H)(Me), CH₂N(C₁₋₄alkyl)₂ (e.g. —CH₂—N(Me)₂) or CH₂heterocyclyl group (e.g. —CH₂-pyrrolidin-1-yl or —CH₂-4-methyl-piperazin-1-yl)).

Suitably R⁴ also represents —COOH, —NH—COO—R⁵ or —CO—NH—R⁶.

In one embodiment R⁴ represents —CO—NH—R⁶ (e.g. —CO—NH-piperidin-1-yl or —CO—NH-morpholin-4-yl). In a further embodiment R⁴ represents —CO—NH—R⁶ (e.g. —CO—NH-morpholin-4-yl).

In one embodiment, R⁵ represents CH₂-piperidin-4-yl.

In one embodiment, R⁶ represents an aliphatic heterocyclyl group (e.g. piperidin-1-yl or morpholin-4-yl) or an aryl group (e.g. phenyl optionally substituted by one or more halogen atoms, e.g. 2,4-difluorophenyl or an optionally substituted C₁₋₆alkylamino, e.g. 4-methylaminomethylphenyl or 4-ethylaminomethylphenyl) or an -aryl-CH₂-aliphatic heterocyclyl group (e.g. -phenyl-CH₂-piperidinyl optionally substituted by a hydroxy group or -phenyl-CH₂-pyrrolidinyl optionally substituted by a hydroxy group).

In another aspect the present invention provides one or more chemical entities selected from compounds of formula (Ib):

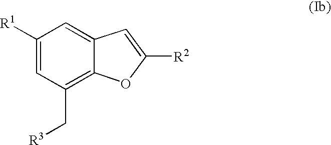

wherein
R¹ is hydrogen, Cl, Br, or CN;
R² is isopropyl, propyl or C₃₋₆ cycloalkyl;

### R³ is

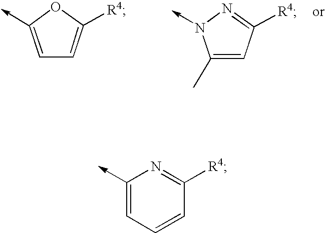

R⁴ is CO₂H, NHCO₂R⁵, CONR^{6a}R^{6b}, CONHSO₂R⁷, NHCONR⁸R⁹, NHCOR¹⁰, imidazole or tetrazole;
or R⁴ is an imidazole ring fused to give an optionally substituted bicyclic or tricyclic ring system;
R⁵ is C₂₋₆ alkyl or optionally substituted CH₂aliphatic heterocycle;
R^{6a} is hydrogen; and
R^{6b} is hydrogen; NR¹¹R¹²; C₁₋₆alkyl optionally substituted by NR¹¹R¹²; phenyl optionally substituted by halogen, CH₂OH, CH₂NR¹¹R¹², or optionally substituted CH₂aliphatic heterocycle; or optionally substituted (CH₂)ₙaliphatic heterocycle wherein n is 0, 1 or 2;
or R^{6a} and R^{6b} together with the nitrogen atom to which they are attached form an optionally substituted aliphatic heterocycle;
R¹⁰ is C₃₋₆cycloalkyl optionally substituted by OH; C₁₋₆alkyl; or optionally substituted (CH₂)ₘaliphatic heterocycle wherein m is 0, or 1; or

### R¹⁰ is

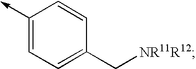

R¹¹ is hydrogen or C₁₋₄alkyl; and
R¹² is hydrogen or C₁₋₄alkyl;
or derivatives thereof.

In one aspect R² is isopropyl or C₃₋₆cycloalkyl.

In one aspect R³ is

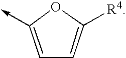

In another aspect R³ is

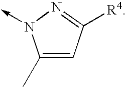

In a further aspect R³ is

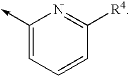

### R⁴ is CO₂H, NHCO₂R⁵, CONR^{6a}R^{6b}, NHCOR¹⁰;

or R⁴ is an imidazole ring fused to give an optionally substituted bicyclic or tricyclic ring system;

In one aspect R⁴ is CO₂H, NHCO₂R⁵, CONR^{6a}R^{6b}, NHCOR¹⁰ or optionally substituted benzimidazole. In a further aspect R⁴ is CO₂H.

Compounds of formula (I) include the compounds of examples 1 to 72 and derivatives thereof.

Particular Examples of compounds of Formula (I) include the compounds of Examples 1, 3, 7, 14, 15, 16, 18, 19, 22, 24, 27, 28 and 29.

A particular example of a compound of formula (I) is 1-{[5-chloro-2-(1-methylethyl)-1-benzofuran-7-yl]methyl}-5-methyl-N-4-morpholinyl-1H-pyrazole-3-carboxamide or a derivative thereof, particularly a pharmaceutically acceptable derivative thereof.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

The term “aliphatic heterocyclyl” or “aliphatic heterocycle” as a group or as part of a group means an aliphatic five or six membered ring which contains 1 or 2 heteroatoms selected from nitrogen, oxygen or sulfur and is unsubstituted or substituted by, for example, up to three substituents, preferably one or two substituents. Unless otherwise stated, the aliphatic heterocycle may be linked by a carbon or nitrogen atom.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

Compounds of formula (I) can be prepared as set forth in the following scheme and in the examples. The following processes form another aspect of the present invention.

For example, compounds of formula (Ia) wherein R³ represents

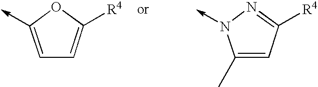

and R⁴ is CO₂H hereinafter referred to as compounds of formula (Ia)^{a} and (Ia)^{b}, respectively, may be prepared by the general route shown in Scheme I below:

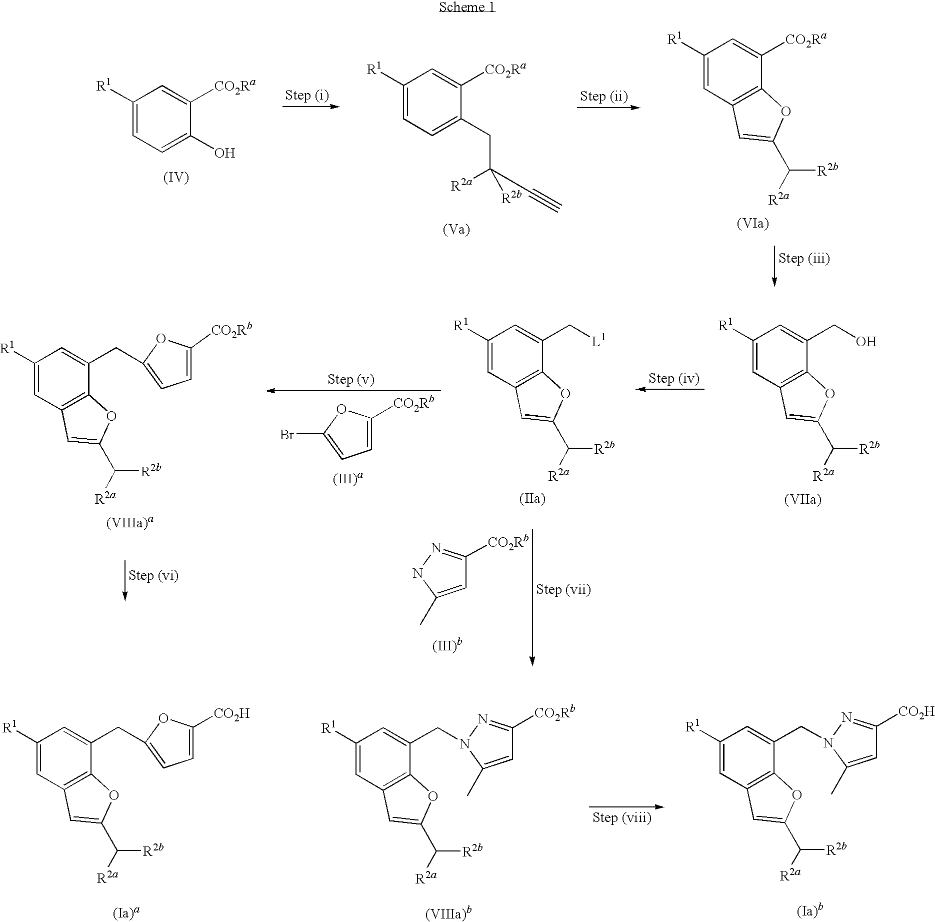

wherein L¹ is a halo group (selected for example from bromo and iodo) or mesylate or tosylate; R¹, R^{2a} and R^{2b} are as defined for compounds of Formula (Ia); and R^{a} and R^{b} are protecting groups selected from alkyl groups (e.g. methyl or ethyl).

Step (i) typically comprises reacting a compound of formula (IV) with 2-methyl-3-butyn-2-ol with suitable reagents such as trifluoroacetic anhydride and 1,8-diazabicyclo[5.4.0]undec-7-ene (DBU) in acetonitrile in the presence of CuCl₂ under suitable conditions, such as at −10° C. under argon.

Step (ii) typically comprises the use of suitable reagents such as caesium fluoride in N,N-diethylaniline at suitable conditions, such as at 180° C. under argon.

Step (iii) typically comprises the use of a suitable reducing agent such as lithium aluminium hydride in the presence of a suitable solvent such as anhydrous tetrahydrofuran (THF).

Step (iv) typically comprises the conversion of the alcohol moiety into a leaving group, for example by reaction with a reagent such as phosphorus tribromide in the presence of a suitable solvent such as dichloromethane/hexane at suitable conditions, such as room temperature.

Step (v) typically comprises incubation of a compound of formula (IIa) with a mixture of dibromoethane, zinc dust and tetrahydrofuran (THF) at 60° C. under argon prior to reaction with a compound of formula (III)^{a} in the presence of a suitable catalyst such as tetrakis(triphenylphosphine)palladium(0).

Step (vii) typically comprises reaction of a compound of formula (IIa) with a compound of formula (III)^{b} in the presence of a base, such as potassium carbonate, in a solvent, such as N,N-dimethylformamide (DMF), at ambient temperature.

Steps (vi) and (viii) typically comprise a deprotection reaction which will be known to the skilled person. Conditions for the deprotection of an ester to give the corresponding carboxylic acid are known to those skilled in the art and include heating in the presence of a suitable base, e.g. aqueous sodium hydroxide, in a solvent e.g. an alcohol.

Compounds of Formula (III)^{a} and (III)^{b} are commercially available made by known transformations from commercially available intermediates.

Compounds of formula (IV) are commercially available, or may be readily made by known transformations from commercially available intermediates.

Compounds of formula (I) wherein R³ is

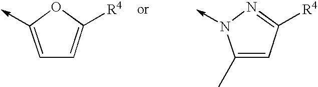

and R⁴ is CO₂H, hereinafter referred to as compounds of formula (I)^{a} and (I)^{b}, respectively, may be prepared by the general route shown in Scheme II below:

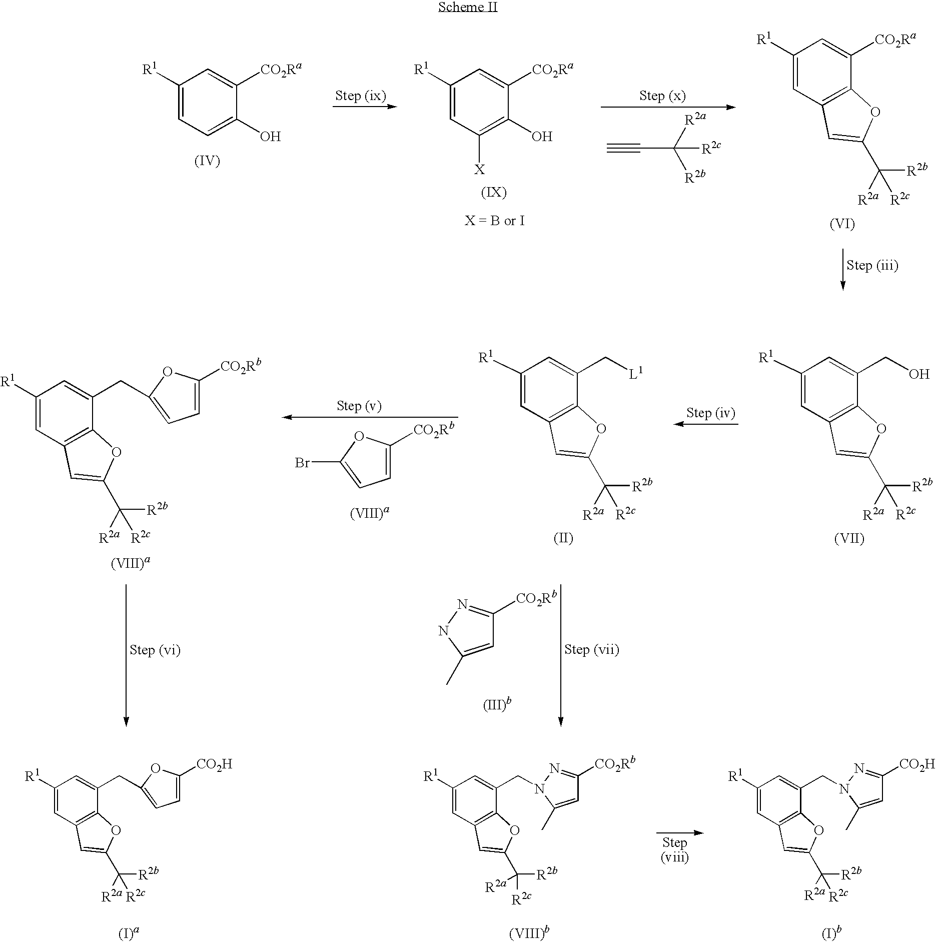

wherein L¹ is a halo group (selected for example from bromo and iodo), or mesylate or tosylate; R¹, R^{2a}, R^{2b} and R^{2c} are as defined for compounds of formula (I); and R^{a} and R^{b} are protecting groups selected from alkyl groups (e.g. methyl or ethyl).

Step (ix) typically involves reacting a compound of formula (IV) with a halogenating reagent, for example N-bromosuccinimide (NBS) or N-iodosuccinimide (NIS).

Step (x) typically involves reacting a compound of formula (IX) with an appropriately substituted acetylene in a solvent such as N,N-dimethylformamide (DMF) in the presence of a palladium and copper catalyst, for example (Ph₃P)₂PdCl₂ and CuI, in the presence of a base, for example triethylamine, at an appropriate temperature, for example room temperature or an elevated temperature, for example 70-80° C.

Remaining steps (iii), (iv), (v), (vi), (vii) & (viii) may be performed using the procedures described above for Scheme I.

Compounds of Formula (III)^{a}, (III)^{b} and (IV) are commercially available or may be made by known transformations from commercially available intermediates.

Substituted acetylenes are commercially available, or may be made by known transformations from commercially available intermediates.

Alternatively, compounds of formula (I) wherein R³ is

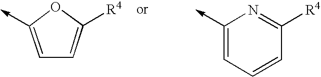

and R⁴ is CO₂H, hereinafter referred to as compounds of Formula (I)^{a} and (I)^{c} respectively may be prepared according to the route described in Scheme III.

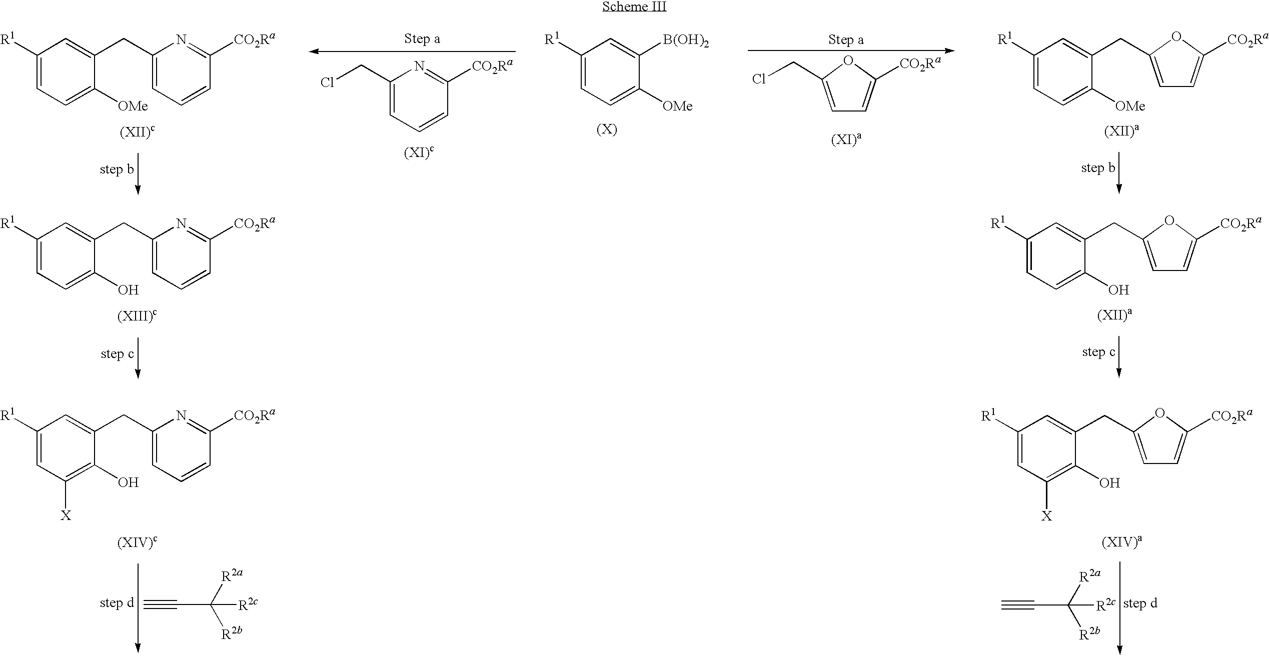

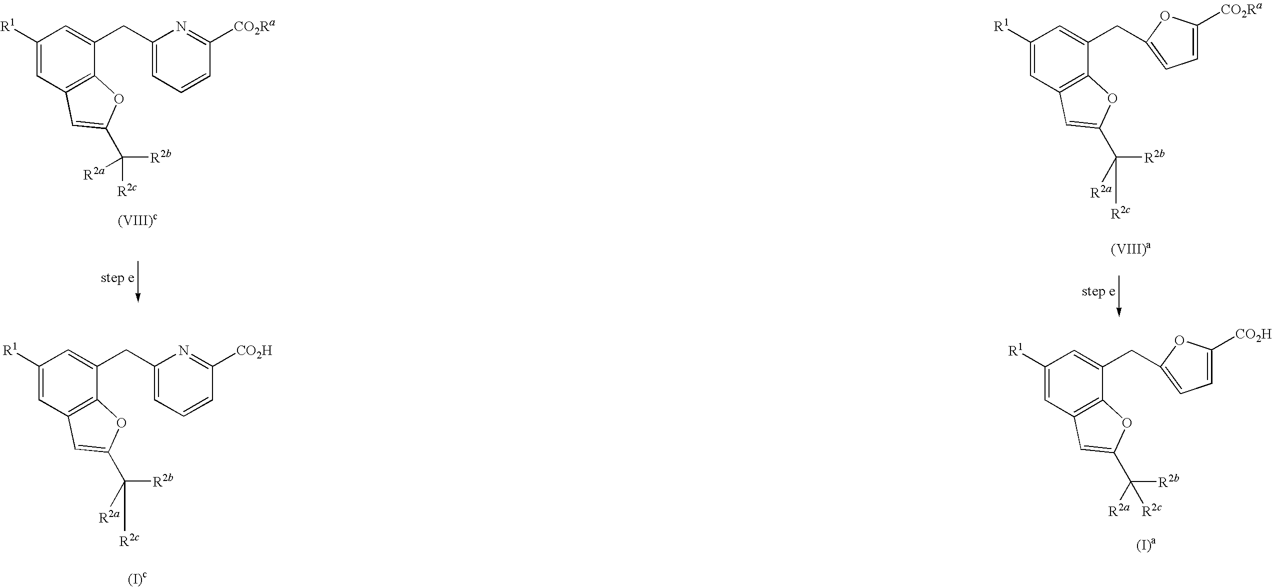

wherein X is bromo or iodo, R¹, R^{2a}, R^{2b} and R^{2c} are as defined for compounds of Formula (I); and R^{a} is a protecting group selected from alkyl groups (e.g. methyl or ethyl).

Step (a) typically comprises the reaction of a compound of Formula (X) with a compound of Formula (XI)^{a} or (XI)^{c} in a suitable solvent, such as ethanol/toluene, in the presence of a palladium catalyst, for example Pd(PPh₃)₄ and a base, such as potassium carbonate, at an appropriate temperature, for example 90° C.

Step (b) typically involves reaction of a compound of formula (XII)^{a} or (XII)^{c} with boron tribromide in a suitable solvent, such as dichloromethane, at an appropriate temperature, for example room temperature.

Step (c) typically involves reacting a compound of formula (XIII)^{a} or (XIII)^{c} with a halogenating reagent, for example N-bromosuccinimide (NBS) or N-iodosuccinimide (NIS) in a suitable solvent, e.g. DMF.

Step (d) typically involves reaction of a compound of Formula (XIV)^{a} or (XIV)^{c} with an appropriately substituted acetylene in a solvent such as N,N-dimethylformamide in the presence of a palladium and copper catalyst, for example (Ph₃P)₂PdCl₂ and CuI, and a base, for example triethylamine at an appropriate temperature, for example room temperature or an elevated temperature, for example 70-80° C.

Deprotection step (e) typically comprises methods which will be known to the skilled person. Conditions for the deprotection of an ester such as a compound of formula (VIII)^{a} or (VIII)^{c} to give the corresponding carboxylic acid of formula (I)^{a} or (I)^{c} are known to those skilled in the art and include heating in the presence of a suitable base, e.g. aqueous sodium hydroxide, in a solvent e.g. an alcohol.

Compounds of the Formula (XI)^{a} and (XI)^{c} are commercially available or can be prepared from the corresponding alcohol derivative by procedures known to those skilled in the art, for example by treatment of the alcohol derivative with thionyl chloride, or by the methods described in the Examples.

Alternatively compounds of Formula (I)^{a} and (I)^{c} may be prepared in accordance with the route outlined in Scheme IV:

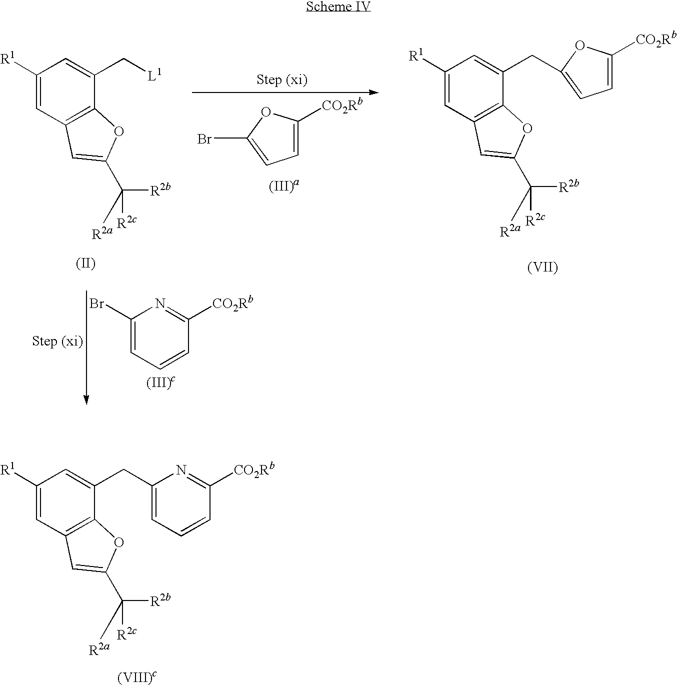

wherein L¹ is a halo group (selected for example from bromo and iodo), or mesylate or tosylate; R¹, R^{2a}, R^{2b} and R^{2c} are as defined for compounds of formula (I); and R^{b} is a protecting group selected from alkyl groups (e.g. methyl or ethyl).

Step (xi) typically involves activating a compound of formula (II), for example by reacting with zinc in the presence of chlorotrimethylsilane (TMSCl), in a solvent, such as tetrahydrofuran at a suitable temperature, for example room temperature, followed by reaction with a compound of formula (III)^{a} or (III)^{c} in the presence of a palladium catalyst, for example Pd(PPh₃)₄ at an appropriate temperature, for example 50° C.

Compounds of Formula (III)^{c} are commercially available.

Compounds of Formula (VIII)^{a} may be converted to a compound of Formula (I)^{a} and compounds of Formula (VIII)^{c} may be converted to a compound of Formula (I)^{c} by deprotection of the ester group as described for example in Step e of Scheme III above.

It will be recognised to those skilled in the art that the compounds of formula (I) can be derived from the carboxylic acid intermediates of formula (I)^{a} and (I)^{b}. Compounds of formula (I) wherein R⁴ is CONR^{6a}R^{6b} or CONHSO₂R⁷ can be prepared by activation of the carboxylic acid, for example by forming the acid chloride (for example by reaction of the carboxylic acid with thionyl chloride) followed by reaction with an amine or a sulfonamide respectively. Compounds of formula (I) wherein R⁴ is NHCO₂R⁵ may be accessed by using the Curtius reaction (P. A. S. Smith, Org. React. 3, 337-449 (1946) and J. H. Saunders, R. J. Slocombe, Chem. Rev. 43, 205 (1948)). Compounds of Formula (I) wherein R⁴ is NHCOR¹⁰ may also be accessed by using the aforementioned Curtius reaction followed by deprotection of the resulting carbamate and reaction of the amine with a carboxylic acid derivative such as an acid chloride in accordance with known methods. Compounds of Formula (I) wherein R⁴ is NHCONR⁸R⁹ may also be accessed by using the aforementioned Curtius reaction followed by deprotection of the resulting carbamate and reaction of the amine with an isocyanate or 1,1′-carbonyldiimidazole in accordance with known methods. It will be recognised to those skilled in the art that a carboxylic acid group may be converted to an imidazole group by a sequence of well known functional group transformations such as those described in the Examples and those described in A. R. Katritzky, C. W. Rees ‘Comprehensive Heterocyclic Chemistry’, Pergamon (1984). Tetrazoles may be formed from carboxylic acids by converting the carboxylic acid to the primary amides, for example by reaction with oxalyl chloride followed by ammonia, followed by dehydration of the amide to the nitrile, for example by heating in phosphorous oxychloride, followed by reaction with azide.

Compounds of Formula (I) wherein R⁴ is an imidazole moiety fused to give an optionally substituted bicyclic or tricyclic ring system [hereinafter referred to as compounds of formula 1^{d}] may be prepared from compounds of formula (XV) following the methods described in, for example, A. Czarny et al, J. Het. Chem., 1996, 33(4), 1393-1398 and according to the following Scheme V:

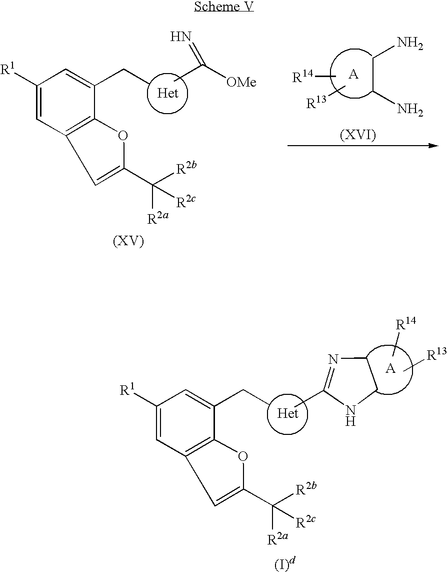

wherein R¹, R^{2a}, R^{2b} and R^{2c} are as defined for compounds of formula (I); Het represents the ring systems as defined in R³, A represents e.g. phenyl, pyridine, pyridazine, pyrazine, pyrimidine, quinoline, or thiophene, particularly phenyl, and R¹³ and R¹⁴ each represent hydrogen or a substituent.

Suitable reaction conditions for the preparation of a compound of formula (I)^{d} include heating the compounds of formula (XV) with compounds of formula (XVI) together in a suitable solvent e.g. ethanol.

Compounds of formula (XV) may be prepared from the corresponding carboxylic acid of formula (I)^{a}, (I)^{b} or (I)^{c} by known methods. Suitable methods include the reaction of a compound of formula (I)^{a}, (I)^{b} or (I)^{c} with thionyl chloride then ammonia, then phosphorus oxychloride, then sodium methoxide in methanol.

Alternatively, for example, compounds wherein R⁴ is benzimidazolyl may typically be prepared by reacting a compound of formula (I)^{a}, (I)^{b} or (I)^{c} with 1,2-phenylenediamine or a suitably substituted analogue, N-(3-dimethylaminopropyl)-N′-ethylcarbodiimide hydrochloride and 1-hydroxybenzotriazole (HOBT) in the presence of a suitable solvent such as dichloromethane, followed by dehydration according to standard conditions known to the skilled person, for example, according to procedures described in the Examples.

Diamines of formula (XVI) are commercially available, or may be prepared by known methods.

Compounds of formula (I) wherein R⁴ is benzimidazolyl [referred to as compounds of formula (I)^{d}] may also be prepared from the reaction of a diamine of formula (XVI) with a compound of formula (XVII):

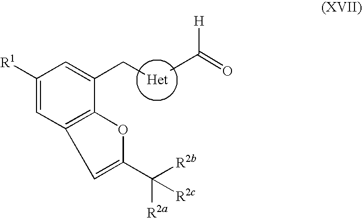

wherein R¹, R^{2a} and R^{2b} and R^{2c} are as defined for compounds of formula (I) and Het represents the ring systems as defined by R³ as defined for compounds of formula (I).

Compounds of formula (XVII) may be prepared from compounds of formula (I)^{a}, (I)^{b} or (I)^{c} by known methods, for example by reaction of a compound of formula (I)^{a}, (I)^{b} or (I)^{c} with lithium aluminium hydride in a suitable solvent, e.g. tetrahydrofuran to give the corresponding methanol, followed by conversion to the corresponding carbaldehyde using Dess-Martin periodinane.

Compounds of formula (I) wherein R⁴ is benzimidazole (i.e. compounds of formula (I)^{d}) may be functionalised on the benzimidazole ring using methods described in the Examples and in the Scheme VI below:

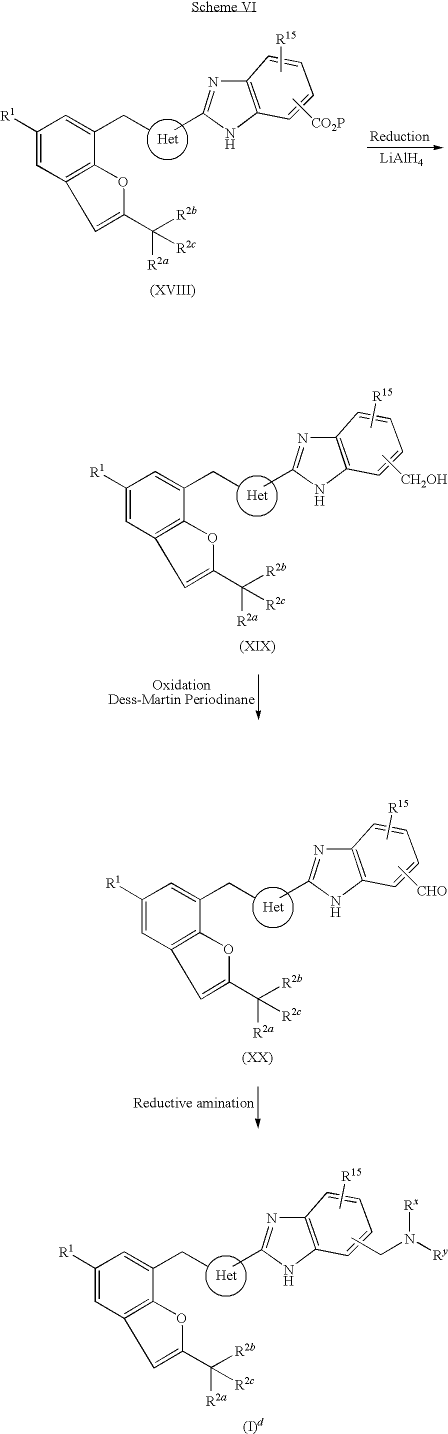

wherein P is a protecting group; R¹, R^{2a}, R^{2b} and R^{2c} are as defined for compounds of formula (I), Het represents the ring systems as defined in R³, of compounds of formula (I), R¹⁵ is hydrogen or a substituent, R^{x} and R^{y} are independently selected from hydrogen and optionally substituted C₁₋₄alkyl, or R^{x} and R^{y} together with the nitrogen atom to which they are attached form a heterocyclyl ring optionally containing another heteroatom selected from O, NH, NC₁₋₄alkyl, or S.

Compounds of formula (I) wherein R⁴ is CONR^{6a}R^{6b} when R^{6a} is hydrogen and R^{6b} is optionally substituted phenyl may be functionalised in accordance with the methods outlined in the Scheme VI above.

Compounds of formula (I) wherein R⁴ is NHCO₂R⁵ may typically be prepared by the aforementioned Curtius reaction for example by reacting a compound of formula (I)^{a}, (I)^{b} or (I)^{c} with an appropriate alcohol in the presence of toluene, triethylamine and diphenylphosphoryl azide at a suitable temperature such as 80° C. It will be appreciated by those skilled in the art that when the alcohol contains certain functional groups that a protecting group may be necessary.

Compounds of formula (I) wherein R⁴ is CONR^{6a}R^{6b} may typically be prepared by first reacting a compound of formula (I)^{a} or (I)^{b} with thionyl chloride or oxalyl chloride in dichloromethane and/or N,N-dimethylformamide to prepare a carbonyl chloride derivative, followed by reaction with an appropriate amino compound (such as an amine, aniline, aminoaryl or heterocyclic derivative thereof) in a suitable solvent such as dichloromethane and a suitable base such as triethylamine and/or pyridine.

Accordingly the present invention also provides a process for the preparation of a compound of formula (I) or a derivative thereof:

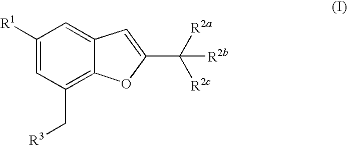

wherein
R¹ is hydrogen, halogen, CF₃, CN, SO₂CH₃ or CH₃;
R^{2a} is hydrogen, C₁₋₄ alkyl, or CF₃;
R^{2b} is C₁₋₄ alkyl or CF₃; and
R^{2c} is hydrogen or CH₃;
or R^{2a} and R^{2b} together with the carbon atom to which they are attached form a C₃₋₆ cycloalkyl group, and R^{2c} is hydrogen;

### R³ is:

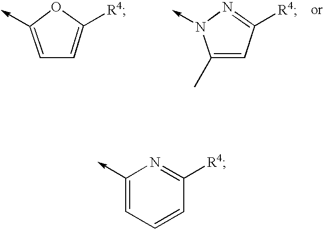

R⁴ is CO₂H, NHCO₂R⁵, CONR^{6a}R^{6b}, CONHSO₂R⁷, NHCONR⁸R⁹, NHCOR¹⁰, imidazole or tetrazole;
or R⁴ is an imidazole ring fused to give an optionally substituted bicyclic or tricyclic ring system;
R⁵ is C₂₋₆ alkyl, optionally substituted CH₂phenyl or optionally substituted CH₂aliphatic heterocycle;
R^{6a} is hydrogen; and
R^{6b} is hydrogen; NR¹¹R¹²; C₁₋₆alkyl optionally substituted by NR¹¹R¹²; phenyl optionally substituted by halogen, CH₂OH, CH₂NR¹¹R¹², or optionally substituted CH₂aliphatic heterocycle; or optionally substituted (CH₂)ₙaliphatic heterocycle wherein n is 0, 1 or 2;
or R^{6a} and R^{6b} together with the nitrogen atom to which they are attached form an optionally substituted aliphatic heterocycle;
R⁷ is C₁₋₄alkyl, phenyl or heteroaryl;
R⁸ is hydrogen or C₁₋₄alkyl;
R⁹ is C₁₋₄alkyl;
R¹⁰ is C₃₋₆cycloalkyl optionally substituted by OH; C₁₋₆alkyl; or optionally substituted (CH₂)ₘaliphatic heterocycle wherein m is 0, or 1; or

### R¹⁰ is

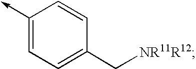

R¹¹ is hydrogen or C₁₋₄alkyl; and
R¹² is hydrogen or C₁₋₄alkyl;
comprising:
converting a compound of formula (II):

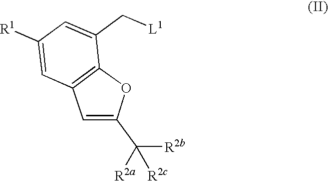

wherein L¹ is a leaving group selected for example from bromo, iodo, tosyl and mesyl; and
R¹, R^{2a}, R^{2b} and R^{2c} are as defined for compounds of formula (I);
to a compound (VIII):

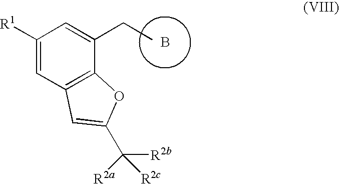

Wherein R¹, R^{2a}, R^{2b} and R^{2c} are as defined for compounds of formula (I) and B is:

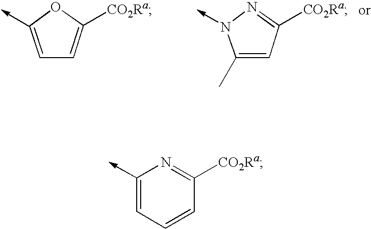

wherein R^{a} is a protecting group, e.g. methyl or ethyl;
and in any order, and if required;
converting the group CO₂R^{a} to a group R⁴; and
converting one group R⁴ to another group R⁴; and/or
effecting deprotection; and/or
forming a derivative thereof.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T.W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opoids (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO0/26216, WO0/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

In addition to activity at the EP₁ receptor, certain compounds of the present invention and pharmaceutically acceptable derivatives thereof exhibit antagonism of the TP receptor and are therefore indicated to be useful in treating conditions mediated by the action of thromboxane at the TP receptor. Conditions mediated by the action of thromboxane at the TP receptor include renal disorders, asthma, or gastric lesions.

In certain situations it is envisaged that the administration of a compound exhibiting antagonism of TP receptors in combination with a compound exhibiting antagonism of EP₁ receptors may be advantageous.

Certain compounds of the invention are selective for EP₁ over EP₃.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

It will be appreciated to those skilled in the art that where compounds are named as hydrochloride salts the stoichiometry of the isolated reaction products is undetermined due to the nature of their preparation. Compounds have therefore been named as hydrochlorides and denoted as xHCl, where x is 0-3 and represents the stoichiometry of said salt.

### ABBREVIATIONS

AcOH (acetic acid), Bn (benzyl), BOC (tert-butoxy), Bu, Pr, iPr, Me, Et (butyl, propyl, isopropyl, methyl, ethyl), DBU (1,8-diazabicyclo[5.4.0]undec-7-ene), DMSO (dimethyl sulfoxide), DCM/MDC (dichloromethane), DME (ethylene glycol dimethyl ether), DMF (N,N-dimethylformamide), DMP (Dess-Martin periodinane), DPPA (diphenyl phosphoryl azide), EDAC/EDC (N-(3-dimethylaminopropyl)-N′-ethylcarbodiimide), EDTA (ethylenediaminetetraacetic acid), EtOAc (ethyl acetate), EtOH (ethanol), Et₂O (diethyl ether), HOBT/HOBt (1-hydroxybenzotriazole), HPLC (High pressure liquid chromatography), IPA (isopropanol), LCMS (Liquid chromatography/Mass spectroscopy), MDAP (Mass Directed Auto Preparation), MeOH (methanol), ML (mother liquor), NMR (Nuclear Magnetic Resonance (spectrum)), NMP (n-methylpyrrolidone), Ph (phenyl), PhCH₃ (toluene), i-PrOH (isopropanol), pTSA (para-toluene sulfonic acid), RT/Rt (retention time), SM (starting material), SPE (Solid Phase Extraction-silica cartridge chromatography), TBAF (tetrabutylammonium fluoride), TBME (tertiary butyl methyl ether), TEA (triethylamine), TFAA (trifluoacetic anhydride), THF (tetrahydrofuran), s, d, dd, t, q, m, br (singlet, doublet, double doublet, triplet, quartet, multiplet, broad.)

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

The terms “Biotage®”, “Biotage 75” and “Biotage SP4®” when used herein refer to commercially available automated purification systems using pre-packed silica gel cartridges.

### LCMS

The following LCMS conditions were used during the preparation of the examples.

### Software

Waters MassLynx version 4.0 SP2

### Column

The column used is a Waters Atlantis, the dimensions of which are 4.6 mm×50 mm. The stationary phase particle size is 3m.

### Solvents

A: Aqueous solvent=Water+0.05% Formic Acid

B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

All retention times are measured in minutes.

### Preparation of Intermediates

### Description 1

### 1,1-dimethylethyl [trans-4-(ethyloxy)cyclohexyl]carbamate (D1)

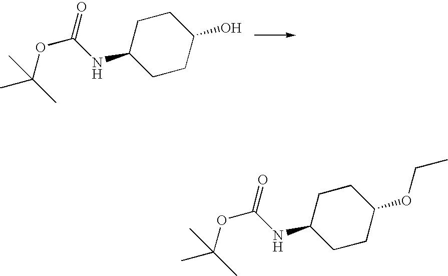

A solution of 1,1-dimethylethyl (trans-4-hydroxycyclohexyl)carbamate (0.300 g, 1.39 mmol) and sodium hydride (0.122 g, 3.07 mmol) in dry THF (5.6 ml) was stirred at room temperature under an atmosphere of argon for 45 minutes. After this time, ethyl iodide (0.111 ml, 1.39 mmol) was added and the reaction solution was stirred at room temperature for 17 hours (overnight). The reaction was monitored by LC-MS. The reaction mixture was cooled to 0° C. and quenched by the slow addition of sodium hydroxide (5 ml, 2M) and ammonium chloride (5 ml, sat. aq solution). The reaction solution was stirred, warming to room temperature over 30 minutes. The organics were extracted using EtOAc (3×50 ml). Brine was added to encourage the separation. The combined organics were dried over magnesium sulfate, filtered and concentrated under reduced pressure to give a cream solid. The solid was chromatographed [SiO₂, EtOAc:Hexane 0-10%] to give 1,1-dimethylethyl [trans-4-(ethyloxy)cyclohexyl]carbamate (0.045 g, 13%).

### Description 2

### trans-4-(ethyloxy)cyclohexanamine (D2)

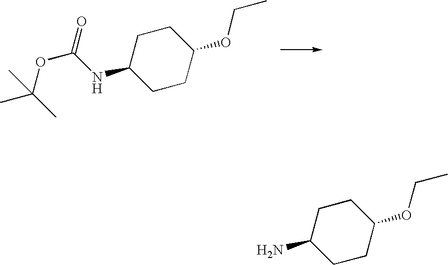

A solution of 1,1-dimethylethyl [trans-4-(ethyloxy)cyclohexyl]carbamate (0.045 g, 0.185 mmol) in hydrochloric acid in dioxane (4M, 1 ml) for 1 hour. The reaction mixture was concentrated under reduced pressure to give trans-4-(ethyloxy)cyclohexanamine (0.037 g).

### Description 3

### Methyl 2-hydroxy-5-(methylsulfonyl)benzoate (D3)

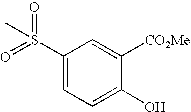

Boron tribromide (30.12 g, 120 mmol) was added carefully to an ice-cooled, stirred solution of methyl 2-(methyloxy)-5-(methylsulfonyl)benzoate (9.76 g, 40 mmol) in dichloromethane (200 ml) producing a gummy precipitate. Stirred for 30 minutes and the solution was poured onto ice and the gummy precipitate dissolved in ethyl acetate/water (200 ml of each). The ethyl acetate and the dichloromethane solutions were dried (magnesium sulphate) combined and evaporated. The residue was dissolved in methanol (150 ml) and sulphuric acid (10 ml) and refluxed under argon for 20 hours. The resulting solution was cooled, evaporated, dissolved in ethyl acetate/water (200 ml of each) and the organic phase washed with saturated sodium bicarbonate, dried (magnesium sulphate) and evaporated to give the title compound as a white solid (7.9 g).

LC/MS: Rt=2.11 min, [M+H]⁺ 231.2

### Description 4

### Methyl 6-(chloromethyl)-2-pyridinecarboxylate (D4)

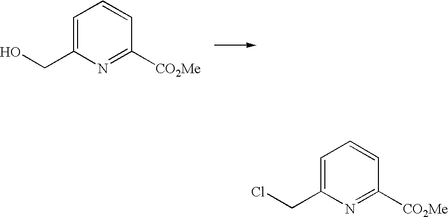

Thionyl chloride (1.785 g, 15 mmol) was added to a solution of methyl 6-(hydroxymethyl)-2-pyridinecarboxylate (2.088 g, 12.5 mmol) in dichloromethane (50 ml) and left at room temperature for one hour. The resulting solution was washed with 1M potassium carbonate solution, dried (magnesium sulphate) and evaporated to give the title compound as a colourless oil (2.32 g).

LC/MS: Rt=2.01 min, [M+H]⁺ 186.1, 188.1

### Description 5

### Ethyl 6-{[5-chloro-2-(methyloxy)phenyl]methyl}-2-pyridinecarboxylate (D5)

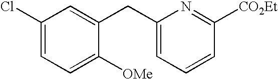

A mixture of 5-chloro-2-methoxyphenylboronic acid (2.33 g, 12.5 mmol), methyl 6-(chloromethyl)-2-pyridinecarboxylate (2.32 g, 12.5 mmol), potassium carbonate (6.9 g, 50 mmol) and tetrakis(triphenylphosphine)palladium(0) (724 mg, 0.625 mmol) in 1:1 ethanol/toluene (100 ml) was stirred and heated at 90° C. under argon for 3 hours. The mixture was cooled, diluted with water (200 ml) and ether (50 ml) and the organic phase dried (magnesium sulphate), evaporated and purified by flash chromatography on a Biotage® column eluting with 1:4 ethyl acetate/hexane. The title compound was isolated as a colourless oil (1.8 g).

LC/MS: Rt=3.19 min, [M+H]⁺ 306.1, 308.1

### Description 6

### Ethyl 5-{[5-chloro-2-(methyloxy)phenyl]methyl}-2-furancarboxylate (D6)

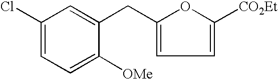

Prepared in a similar manner to above but using ethyl 5-chloromethyl-2-furancarboxylate instead of methyl 6-(chloromethyl)-2-pyridinecarboxylate.

LC/MS: Rt=3.43 min, [M+H]⁺ 295.2, 297.2

### Description 7

### Ethyl 6-[(5-chloro-2-hydroxyphenyl)methyl]-2-pyridinecarboxylate (D7)

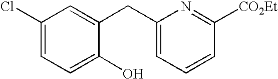

Boron tribromide (4.52 g, 18 mmol) was added carefully to a solution of ethyl 6-{[5-chloro-2-(methyloxy)phenyl]methyl}-2-pyridinecarboxylate (1.8 g, 5.89 mmol) in dichloromethane (25 ml) and left at room temperature for 4 hours. A further (4.52 g, 18 mmol) of boron tribromide were added and after 16 hours the solution was poured onto ice. Ethyl acetate (100 ml) was added and the organic phase was dried (magnesium sulphate) and evaporated to give a pale yellow foam (1.66 g) which was dissolved in ethanol (40 ml) and sulphuric acid (3 ml) and refluxed for 6 hours. The resulting solution was cooled, evaporated, dissolved in ethyl acetate/water (60 ml of each), basified with potassium carbonate and the organic phase dried, (magnesium sulphate) and evaporated to give a white solid which was triturated with 2:1 hexane/ether to give the title compound as a white solid (1.09 g).

LC/MS: Rt=3.07 min, [M+H]⁺ 292.2, 294.2

### Description 8

### Ethyl 5-[(5-chloro-2-hydroxyphenyl)methyl]-2-furancarboxylate (D8)

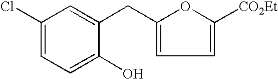

Prepared in a similar manner to above but using ethyl 5-{[5-chloro-2-(methyloxy)phenyl]methyl}-2-furancarboxylate instead of ethyl 6-{[5-chloro-2-(methyloxy)phenyl]methyl}-2-pyridinecarboxylate.

LC/MS: Rt=3.03 min, [M+H]⁺ 281.2, 283.2

### Description 9a

### Methyl 3-bromo-5-chloro-2-hydroxybenzoate (D9a)

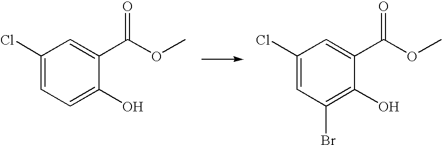

Solution of methyl-5-chlorosalicylate (1.50 g, 8.06 mmol) in dry DMF (16.0 ml) was stirred at room temperature under an atmosphere of argon. N-bromosuccinimide (1.497 g, 8.46 mmol) was added and the solution stirred for 20 hours at room temperature. After this time LC/MS was consistent with product. Solution was diluted with EtOAc and washed with water (×3). Organics were dried over MgSO₄, filtered and concentrated under reduced pressure to give a yellow coloured solid (2.135 g). Residue was used directly for subsequent steps.

### Description 9b

### Methyl 3-bromo-5-chloro-2-hydroxybenzoate (D9b)

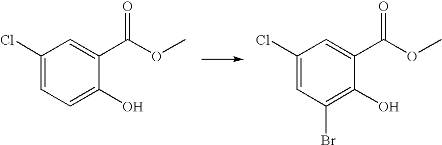

N-Bromosuccinimide (NBS) (47.7601 g) added to a stirred solution of methyl-5-chloro-salicylate (50.0223 g) in DMF (500 mL) at room temperature. Stirred overnight. Solid collected by filtration (batch 1). Further solid formed from filtrate when solid was dried on sinter. Solid collected (batch 2). Filtrate diluted with Et₂O and washed with H₂O. Et₂O layer dried (Na₂SO₄), filtered and concentrated to a low volume—solid collected and washed with cold Et₂O to give batch 3.

Batch 1 yield=34.38 g as a white solid, LCMS Rt=3.42 min, [ES⁻] 265, 267
Batch 2 yield=20.29 g as a white solid, LCMS Rt=3.42 min, [ES⁻] 265, 267
Batch 3 yield=13.38 g as a white solid, LCMS Rt=3.43 min, [ES⁻] 263, 265, 267

Total yield=68.05 g (96%) white solid

### Description 10

### Methyl 5-cyano-2-hydroxy-3-iodobenzoate (D10)

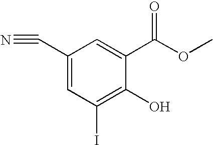

Methyl 2-hydroxy-5-cyanobenzoate (10 g, 0.056 mol) and N-iodosuccinimide (12.7 g) were stirred in DMF (˜100 ml) under argon at room temperature for ˜66 hours (over the weekend). LC/MS showed one peak, product. The cloudy orange mixture was diluted with EtOAc (˜200 ml), HCl (100 ml, 0.1N) added, a lot of solid precipitate—doesn't go into solution (not very soluble in EtOAc)—filtered off to give the title compound as a white solid (˜9.7 g) which contained some DMF (by NMR analysis). The organic phase was washed with water (100 ml), sodium thiosulfate solution (10%, 100 ml), brine (80 ml), dried (MgSO₄) and evaporated to give further title compound as a yellowish solid (˜7.8 g). Confirmed by NMR and LC/MS—some DMF present.

### Description 11

### Ethyl 6-[(5-chloro-2-hydroxy-3-iodophenyl)methyl]-2-pyridinecarboxylate (D1)

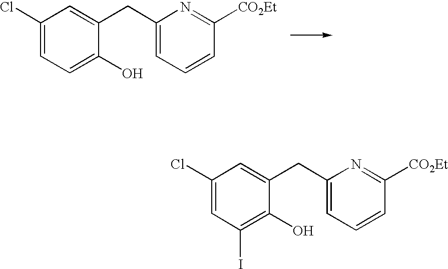

N-Iodosuccinimide (900 mg, 4 mmol) was added to a stirred solution of ethyl 6-[(5-chloro-2-hydroxyphenyl)methyl]-2-pyridinecarboxylate in DMF (6 ml) and stirred for 18 hours. The resulting solution was diluted with water (50 ml) and ethyl acetate (50 ml) and the organic phase washed with 5% sodium thiosulphate solution (50 ml) and water (3×25 ml) then dried (magnesium sulphate), evaporated and purified by flash chromatography on a Biotage® column eluting with 1:4 ethyl acetate/hexane. The title compound was isolated as a white solid (1.34 g).

LC/MS: Rt=3.70 min, [M+H]⁺ 418.0, 420.0

The following compound was prepared in a similar manner to above by reaction of N-iodosuccinimide with the appropriate phenol.

| Description | Structure | Name | LC/MS |
|---|---|---|---|
| D12 | 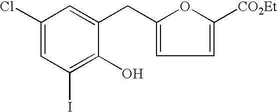 | Ethyl 5-[(5-chloro-2-hydroxy-3-iodophenyl)methyl]-2-furancarboxylate | Rt = 3.37 min,[M + H]⁺ 407.0 |

### Description 13

### Methyl 5-chloro-2-[(1,1-dimethyl-2-propyn-1-yl)oxy]benzoate (D13)

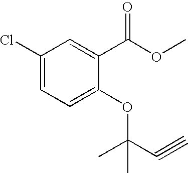

Trifluoroacetic anhydride (6.1 g, 29.06 mmol) was added over 15 minutes to a stirred solution of 2-methyl-3-butyn-2-ol (2.62 g, 31.23 mmol) and DBU (5.52 g, 36.3 mmol) in acetonitrile (40 ml) at −10° C. under argon. The solution was allowed to warm to 0° C. for 30mins then added dropwise to a solution of methyl 5-chloro-2-hydroxybenzoate (4.5 g, 24.13 mmol), DBU (3.29 g, 23.16 mmol) and copper(II) chloride (32 mg, 0.24 mmol) in aceonitrile (40 ml) at −10° C. under argon. The solution was allowed to warm to 0° C., stirred for 2 hours and left in the fridge for 3 days. Saturated ammonium chloride (100 ml) was added and the mixture extracted twice with ether. The combined ether extracts were washed with 2M hydrochloric acid and 2M sodium hydroxide then dried (magnesium sulphate), evaporated and purified by flash chromatography on silica gel eluting with ethyl acetate/hexane (3:97) to give the title compound as a colourless oil (5.35 g).

¹H NMR (CDCl₃) δ: 1.66 (6H, s), 2.58 (1H, s), 3.88 (3H, s), 7.38 (1H, dd), 7.50 (1H, d), 7.75 (1H, d).

### Description 14

### Methyl 5-bromo-2-[(1,1-dimethyl-2-propyn-1-yl)oxy]benzoate (D14)

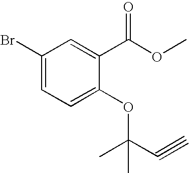

To a solution of 2-methyl-3-butyn-2-ol (6 mL, 61.91 mmol) and DBU (10.8 mL) in dry acetonitrile (80 mL) was added dropwise at −5° C. under argon trifluoroacetic anhydride (TFAA) (8 mL, 57.55 mmol). The solution was warmed to 0° C. and stirred for 30mins, then added dropwise under argon to a solution of methyl 5-bromo-2-hydroxybenzoate (11.14 g, 48.21 mmol), DBU (6.5 mL) and CuCl₂ (64 mg, 0.48 mmol) in dry acetonitrile (80 ml) at −10° C. under argon. The solution was stirred at 2° C. for 2 hours then at room temperature for 15 hours. Saturated ammonium chloride solution (200 mL) was added and the mixture extracted with ether (2×200 mL). The combined ether extracts were washed with 2N hydrochloric acid (50 mL) and 2N sodium hydroxide (50 mL) dried over magnesium sulphate and evaporated down under reduced pressure (u.r.p.). The mixture was dissolved in hexane (400 mL) and the solution washed with 2N NaOH (3×25 mL) to remove the unreacted phenol. The hexane layer was dried over magnesium sulphate and evaporated down under reduced pressure (u.r.p.) to give the straw-coloured oil title compound (12.36 g, 86.3%).

LC/MS [M+1] 297,299.

### Description 15

### Methyl 5-chloro-2-[(1-ethyl-2-propyn-1-yl)oxy]benzoate (D15)

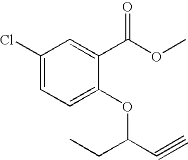

To a solution of 1-pentyn-3-ol (5.35 mL, 62.0 mmol) and DBU (10.8 mL) in dry acetonitrile (80 mL) at −5° C. under argon was added dropwise trifluoroacetic anhydride (TFAA) (8 mL, 57.55 mmol). The solution was warmed to 0° C. and stirred for 30mins, then added dropwise under argon to a solution of methyl 5-chlorosalicylate (9.0 g, 48.23 mmol), DBU (6.5 mL) and CuCl₂ (64 mg, 0.48 mmol) in dry acetonitrile (80 ml) at −10° C. The solution was stirred for 2 hours at 2-3° C. then at room temperature for 15 hours. Saturated ammonium chloride solution (200 mL) was added and the mixture extracted with ether (2×200 mL). The combined extracts were washed with 2N hydrochloric acid (50 mL) and 2N sodium hydroxide (50 mL) dried over magnesium sulphate and evaporated down under reduced pressure (u.r.p.). The mixture was dissolved in hexane (400 mL) and the solution washed with 2N NaOH (3×25 mL) to remove the unreacted phenol. The hexane layer was dried over magnesium sulphate and evaporated down under reduced pressure (u.r.p.) to give the pale buff solid title compound (9.58 g, 78.6%).

LC/MS [M+1] 253, 255.

### Description 16a

### Methyl 5-chloro-2-(1-methylethyl)-1-benzofuran-7-carboxylate (D16a)

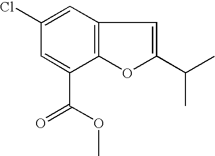

A mixture of methyl 5-chloro-2-[(1,1-dimethyl-2-propyn-1-yl)oxy]benzoate (505 mg, 2 mmol) and cesium fluoride (304 mg, 2 mmol) in N,N-diethylaniline (4 ml) was stirred and heated at 180° C. under argon for one hour. The resulting mixture was cooled, diluted with ether/2M hydrochloric acid and the organic phase washed with 2M hydrochloric acid, dried (magnesium sulphate) and purified by flash chromatography on silica gel eluting with ethyl acetate/hexane (1:49) to give the title compound as a pale yellow oil (96 mg).

LC/MS: Rt=3.74 min, [MH⁺] 253.2, 255.2

### Description 16b

### Methyl 5-chloro-2-(1-methylethyl)-1-benzofuran-7-carboxylate (D16b)

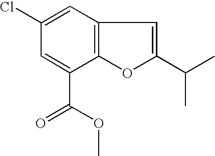

A mixture of methyl 5-chloro-2-[(1,1-dimethyl-2-propyn-1-yl)oxy]benzoate (10.6263 g, 42.07 mmol; may be prepared as described in D1) and cesium fluoride (6.41 g, 42.20 mmol) in N,N-diethylaniline (84 ml) was stirred and heated at 180° C. under argon for six hours then cooled to room temperature. Diluted with ether and 2M hydrochloric acid (˜250 ml each). Layers separate. Organic phase dried (Na₂SO₄), filtered and concentrated. Larger volume than expected (PhNEt₂ still present). Diluted with Et₂O and washed with 2M HCl (×2), dried (Na₂SO₄), filtered and concentrated. The residue was purified by chromatography on silica gel with hexane containing EtOAc (0-5%). Required fractions evaporated to give the title compound. Mixed fractions combined, evaporated and repurified as above. Required fractions evaporated to give the title compound. Yield of title compound 5.2706 g (50%).

LC/MS: Rt 3.64 min, [MH⁺] 253, 255.

### Description 17

### Methyl 5-bromo-2-(1-methylethyl)-1-benzofuran-7-carboxylate (D17)

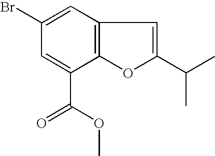

Methyl 5-bromo-2-[(1,1-dimethyl-2-propyn-1-yl)oxy]benzoate (12.36 g, 41.59 mmol) and cesium fluoride (6.32 g, 41.59 mmol) in N,N-diethylaniline (90 ml) were stirred under Ar at 165° C. for 1.5 hours. The mixture was allowed to cool and ether (220 mL) added. The mixture was washed with 2N hydrochloric acid (220 mL then 180 mL) and the organic layer dried over magnesium sulphate and evaporated down under reduced pressure (u.r.p.). The residue was purified by flash chromatography on silica gel (Biotage®) using 2% ethyl acetate in hexane to give the title compound as a pale yellow oil (5.76 g, 46.6%).

LC/MS: [M+1] 297, 299

### Description 18

### Methyl 5-chloro-2-propyl-1-benzofuran-7-carboxylate (D18)

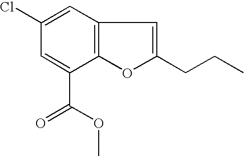

Methyl 5-chloro-2-[(1-ethyl-2-propyn-1-yl)oxy]benzoate (9.58 g, 37.91 mmol) and cesium fluoride (5.76 g, 37.91 mmol) in N,N-diethylaniline (82 ml) were stirred under Ar at 165° C. for 22 hours. The mixture was allowed to cool and ether (200 mL) added. The mixture was washed with 2N hydrochloric acid (200 mL then 160 mL) and the organic layer dried over magnesium sulphate and evaporated down under reduced pressure (u.r.p.). The residue was purified by flash chromatography on silica gel (Biotage®) using 2% ethyl acetate in hexane to give the title compound as a straw coloured oil (7.20 g, 75.2%) which solidified on standing.

LC/MS: [M+1] 253, 255.

### Description 19

### Methyl 5-chloro-2-cyclopropyl-1-benzofuran-7-carboxylate (D19)

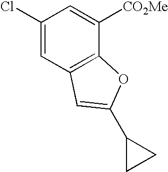

Solution of methyl 3-bromo-5-chloro-2-hydroxybenzoate (2.135 g, 8.06 mmol) in dry DMF (30.0 ml) was stirred at room temperature under an atmosphere of argon. Cyclopropyl acetylene (1.5 ml, 16.12 mmol, 70% in PhCH₃), Et₃N (2.243 ml), CuI (0.140 g, 0.806 mmol) and (Ph₃P)₂PdCl₂ (0.568 g, 0.806 mmol) were added to the stirred solution. The mixture was heated to 75° C. for 3 hours. After this time, LC/MS showed little starting material and potential product. Mixture was allowed to cool to room temperature and left to stand for 72 hours (over weekend). Mixture was diluted with EtOAc and washed with water (×3). Organics were dried over MgSO₄, filtered and concentrated under reduced pressure to give a brown coloured oily solid. The residue was chromatographed [SiO₂, Hexane/EtOAc 0-15%] to give the title compound (1.034 g).

LC/MS Rt=3.55 mins, [MH⁺] 251, 253.

The following compounds were prepared in a similar manner using the appropriate acetylene.

| | | | LCMS |
|---|---|---|---|
| Description | Structure | Name | Data |
| D20 | 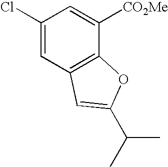 | Methyl 5-chloro-2-(1-methylethyl)-1-benzofuran-7-carboxylate | Rt = 3.70mins [MH⁺]253, 255 |
| D21Heated at75° C. over-night (17 h) | 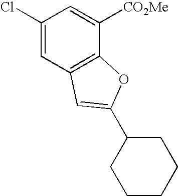 | Methyl 5-chloro-2-cyclohexyl-1-benzofuran-7-carboxylate | Rt 3.97min [M + H]293, 295 |

### Description 22

### Methyl 5-chloro-2-(1,1-dimethylethyl)-1-benzofuran-7-carboxylate (D22)

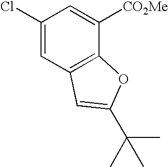

Methyl 3-bromo-5-chloro-2-hydroxybenzoate (3.00 g, 11.30 mmol) dissolved in dry DMF (45 ml). CuI (215 mg, 1.13 mmol), (Ph₃P)₂PdCl₂ (793 mg, 1.13 mmol), 3,3-dimethyl-1-butyne (2.85 ml, 22.60 mmol) and Et₃N (3.15 ml, 22.60 mmol) were added. Reaction solution stirred at room temperature under an atmosphere of argon for 17 hours (overnight). Sample removed for LC/MS analysis—appeared to be complete. Reaction mixture was diluted with EtOAc and washed with water. Aqueous layer washed with further EtOAc. Combined organics were dried over MgSO₄, filtered and concentrated to give a brown/black oil. The residue was chromatographed [SiO₂, Hexane/EtOAc 0-15%] to give the title compound (2.43 g, 81%)

LC/MS Rt=3.76 mins, [M+H] 267, 269.

The following compound was prepared in a similar manner using the appropriate reactants.

| Description | Structure | Name | LC/MS data |
|---|---|---|---|
| D23 | 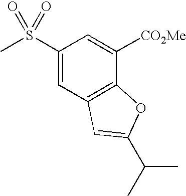 | Methyl 2-(1-methylethyl)-5-(methylsulfonyl)-1-benzofuran-7-carboxylate | Rt 2.86 min, [MH⁺]297.2 |

### Description 24

### Methyl 5-cyano-2-(1-methylethyl)-1-benzofuran-7-carboxylate (D24)

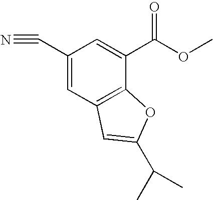

3-Methyl-1-butyne (6.6 ml, 64 mmol) was added to a mixture of methyl 5-cyano-2-hydroxy-3-iodobenzoate (9.7 g, 32 mmol), CuI (609 mg, 3.2 mmol), Pd (PPh₃)₂Cl₂ (2.24 g, 3.2 mmol) and TEA (8.89 ml, 64 mmol) in DMF (60 ml) under argon. The reaction mixture was stirred at room temperature for 18 hours, diluted with water (200 ml) and extracted with ethyl acetate (100ml×3). The combined extracts were washed with water (100ml×2), dried (MgSO₄) and evaporated. The residue was purified on the SP4 using 10-25% of ethyl acetate in hexane to give the title compound as solid (7.4 g).

LC/MS Rt=3.24 min, [MH⁺] 244.2

### Description 25

### 5-Chloro-2-(1-methylethyl)-1-benzofuran-7-yl]methanol (D25)

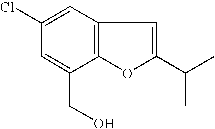

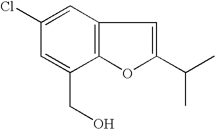

1M Lithium aluminium hydride in THF (6 ml, 6 mmol) was added dropwise to a solution of methyl 5-chloro-2-(1-methylethyl)-1-benzofuran-7-carboxylate (1.5 g, 5.94 mmol) in anhydrous THF (30 ml) over two minutes. The resulting solution was stirred for 15 minutes then quenched by careful addition of 2M hydrochloric acid followed by ether. The organic phase was washed with brine, dried (magnesium sulphate) and evaporated to give the title compound as a colourless oil (1.37 g)

¹H NMR (CDCl₃) δ: 1.34 (6H, d), 1.92 (1H, t), 3.07 (1H, m), 4.95 (2H, d), 6.33 (1H, d), 7.23 (1H, d), 7.38 (1H, d).

### Description 26

### 5-Bromo-2-(1-methylethyl)-1-benzofuran-7-yl]methanol (D26)

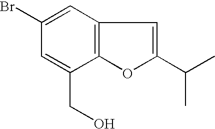

1M Lithium aluminium hydride in THF (20 ml, 20.0 mmol) was added dropwise to a solution of methyl 5-bromo-2-(1-methylethyl)-1-benzofuran-7-carboxylate (5.76 g, 19.38 mmol) in dry THF (100 ml) under argon with ice water cooling. Addition took about seven minutes. The solution was stirred at room temperature for 30 minutes then quenched by careful addition of a few mLs of 2N hydrochloric acid. Ether (400 mL) was added and the solution washed with brine (300 mL). The brine solution was washed with ether (200 mL) and the combined ether layers washed with brine (100 mL), dried over magnesium sulphate) and evaporated down under reduced pressure (u.r.p.) The residue was purified by chromatography on silica gel (Biotage SP4®) using 2-20% EtOAc in hexane to give the title compound as a pale yellow oil (4.10 g, 78.5%).

LC/MS [M+1] 251, 253 (M−18)

### Description 27

### (5-Chloro-2-propyl-1-benzofuran-7-yl)methanol (D27)

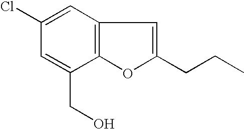

To a stirring solution of methyl 5-chloro-2-propyl-1-benzofuran-7-carboxylate (7.00 g, 27.7 mmol) in dry THF under argon was added with ice cooling 1M lithium aluminium hydride in THF (28 ml, 28.0 mmol). Addition took 20 minutes. The solution was stirred at room temperature for 1 hour then quenched with a few mLs of 2N hydrochloric acid. Ether (550 mL) was added and the mixture washed with brine (400 mL) The brine solution was extracted with ether (250 mL) and the combined ether layers washed with brine (150 mL), dried over magnesium sulphate and evaporated down under reduced pressure (u.r.p.). The residue was purified by chromatography on silica gel (Biotage SP4®) using 2-20% EtOAc in hexane to give the title compound as a pale yellow oil (5.30 g, 85.2%).

LC/MS [M+1] 207, 209.

### Description 28

### (5-Chloro-2-cyclopropyl-1-benzofuran-7-yl)methanol (D28)

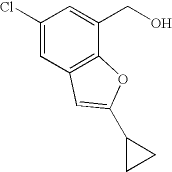

Solution of methyl 5-chloro-2-cyclopropyl-1-benzofuran-7-carboxylate (1.034 g, 4.14 mmol) in dry THF (15 ml) was stirred at 0° C. under an atmosphere of argon. LiAlH₄ (4.14 ml, 4.14 mmol, 1M in THF) was added dropwise to the stirred solution. Solution was stirred at 0° C. for ¼ hour. Water was added dropwise to the solution to quench the LiAlH₄. Solution was diluted with EtOAc and washed with water (×2). Organc is were dried over MgSO₄, filtered and concentrated under reduced pressure to give a yellow oil. (0.885 g)

LC/MS Rt=2.94 mins [MH⁺] 223

The following compounds were prepared in a similar manner using the appropriate ester derivative

| description | Structure | Name | LC/MS data |
|---|---|---|---|
| D29 | 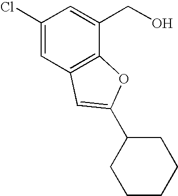 | (5-Chloro-2-cyclohexyl-1-benzofuran-7-yl)methanol | Rt 3.52 min, [M + H]247, 249 |
| D30 | 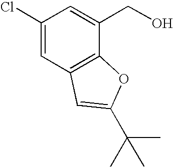 | [5-Chloro-2-(1,1-dimethylethyl)-1-benzofuran-7-yl]methanol | Rt 3.21 min |

### Description 31

### 7-(Hydroxymethyl)-2-(1-methylethyl)-1-benzofuran-5-carbonitrile (D31)

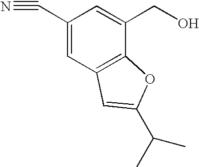

Methyl 5-cyano-2-(1-methylethyl)-1-benzofuran-7-carboxylate (7.22 g, 29.7 mmol) was dissolved in tetrahydrofuran (100 ml) cooled to −10° C. and LiAlH₄ (14.5 ml, 1 M in Et₂O, 14.5 mmol) was slowly added under argon. The reaction mixture was kept cold for ½ h, then warmed to room temperature and stirred for ½ hour. More LiAlH₄ (2.9 ml) was added and the mixture was stirred for a further ½ hour. Quenched with water (200 ml), diluted with ethyl acetate, the solid formed was filtered on celite and the filtrate extracted with ethyl acetate. The combined organics were dried (MgSO₄) and evaporated; the residue was purified on the Biotage SP4® using 10-25% of ethyl acetate in hexane to afford the title compound as yellow solid (4.5 g).

LC/MS Rt=2.81 min, [MH⁺] 216.2

### Description 32

### [2-(1-Methylethyl)-5-(methylsulfonyl)-1-benzofuran-7-yl]methanol (D32)

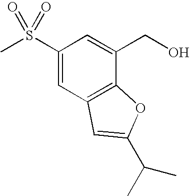

0.5 ml of 2.3M LiAlH₄ (1.15 mmol) were added to a stirred solution of 258 mgs (0.87 mmol) of methyl 2-(1-methylethyl)-5-(methylsulfonyl)-1-benzofuran-7-carboxylate in 5 ml of dry THF under argon. Stirred for 30 minutes then quenched by careful addition of 10 ml 2M NaOH followed by 30 ml of EtOAc. Organics dried (MgSO4) and evaporated to give the title compound (230 mg) as a yellow oil.

LC/MS Rt=2.44 min, [MH⁺] 269.2.

### Description 33

### 7-(Bromomethyl)-5-chloro-2-(1-methylethyl)-1-benzofuran (D33)

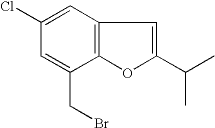

Phosphorus tribromide (1.54 g, 5.68 mmol) was added to a solution of 5-chloro-2-(1-methylethyl)-1-benzofuran-7-yl]methanol (1.28 g, 5.68 mmol; may be prepared as described in D3) in dichloromethane/hexane (1:1, 30 ml) and left at room temperature for one hour. The solution was washed with water, dried (magnesium sulphate), evaporated and purified by flash chromatography on silica gel eluting with ethyl acetate/hexane (1:49) to give the title compound as a white solid (880 mg).

¹H NMR (CDCl₃) δ: 1.36 (6H, d), 3.10 (1H, m), 4.70 (2H, s), 6.33 (1H, d), 7.21 (1H, d), 7.39 (1H, d).

### Description 34

### 7-(Bromomethyl)-5-bromo-2-(1-methylethyl)-1-benzofuran (D34)

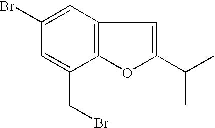

To a solution of 5-Bromo-2-(1-methylethyl)-1-benzofuran-7-yl]methanol (4.10 g, 15.23 mmol) in DCM and hexane (1:1, 50 mL) was added phosphorus tribromide (0.74 mL, 7.87 mmol) and the solution stirred at room temp. under argon for 4 hours. The solution was washed with water (25 mL), dried over magnesium sulphate and evaporated down under reduced pressure (u.r.p.). The residue was purified by chromatography on silica gel using 0-4% EtOAc in hexane to give the title compound (3.40 g, 67.2%) as a pale cream solid.

### Description 35

### 7-(Bromomethyl)-5-chloro-2-propyl-1-benzofuran (D35)

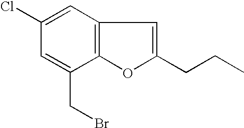

To a stirring solution of (5-chloro-2-propyl-1-benzofuran-7-yl)methanol (5.30 g, 23.59 mmol) in CH₂Cl₂ (65 mL) was added phosphorus tribromide (2.22 mL, 23.59 mmol) and the solution stirred under argon for 3.5 hours. It was washed with water (65 mL), dried over magnesium sulphate and evaporated down under reduced pressure (u.r.p.). The residue was purified by chromatography on silica gel (Biotage SP4®) using 0-4% EtOAc in hexane to give the title compound as a pale buff oil (1.70 g, 25.1%).

### Description 36

### 5-Chloro-7-(chloromethyl)-2-cyclopropyl-1-benzofuran (D36)

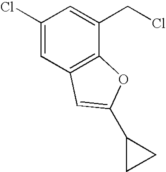

Solution of (5-chloro-2-cyclopropyl-1-benzofuran-7-yl)methanol (0.885 g, 3.99 mmol) in SOCl₂ (0.873 ml, 11.96 mmol) and DCM (8.0 ml) was stirred at room temperature for 2 hours. After one further hour stirring at room temperature LC/MS showed predominantly product. Solvent was removed under reduced pressure and residue used directly in subsequent steps.

LC/MS Rt=3.67 mins

The following compounds were prepared in a similar manner using the appropriate starting material.

| Description | Structure | Name | LC/MS data |
|---|---|---|---|
| D37 | 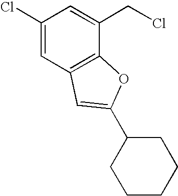 | 5-Chloro-7-(chloromethyl)-2-cyclohexyl-1-benzofuran | Rt 4.14 min |
| D38 | 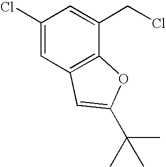 | 5-Chloro-7-(chloromethyl)-2-(1,1-dimethylethyl)-1-benzofuran | Rt 3.91 min |

### Description 39

### 5-Chloro-2-cyclohexyl-7-(iodomethyl)-1-benzofuran (D39)

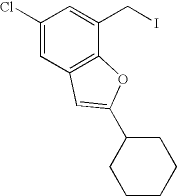

5-Chloro-7-(chloromethyl)-2-cyclohexyl-1-benzofuran dissolved in acetone. NaI added. Reaction stirred at 50° C. under an atmosphere of argon for 1 hour. Sample removed for LC/MS analysis. Heated to 60° C. under argon for 10 minutes. Reaction complete—no starting material present by LC/MS, new peak at Rt 4.32 min. Reaction mixture (dark orange)—some Me₂CO appeared to have evaporated off—cooled to room temperature. Diluted with Et₂O, filtered (solid removed) and concentrated to give the title compound (747 mg, 88%) as a brown solid.

LC/MS Rt=4.26 mins

### Description 40

### [2-(1-methylethyl)-5-(methylsulfonyl)-1-benzofuran-7-yl]methyl Methanesulfonate (D40)

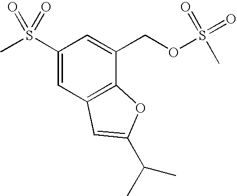

Methanesulphonyl chloride (MsCl) (115 mg, 1 mmol) was added to a solution of Et₃N (152 mg, 1.5 mmol) and [2-(1-methylethyl)-5-(methylsulfonyl)-1-benzofuran-7-yl]methanol (230 mg, 0.86 mmol) in DCM (5 ml). Left at room temperature for 30 minutes then diluted with DCM (20 ml) and washed with water (20 ml). Dried (MgSO₄) and evaporated to give the title compound (295 mg) as a yellow oil.

LC/MS Rt 2.75 min.

### Description 41

### [5-Cyano-2-(1-methylethyl)-1-benzofuran-7-yl]methyl Methanesulfonate (D41)

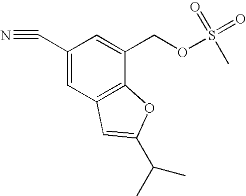

7-(hydroxymethyl)-2-(1-methylethyl)-1-benzofuran-5-carbonitrile (˜4.5 g, 0.021 mol) was dissolved in DCM (˜70 ml), cooled to 0° C., TEA (3.49 ml) added followed by methane sulfonyl chloride (1.95 ml, 0.025 mol). The solution (yellow) was left at 0° C. for 15 minutes then warmed to room temperature ans stirred for 40 minutes. LC/MS analysis indicated 94% product. Water (˜40 ml) added, acidified with 2M HCl, extracted with more DCM (˜70 ml). Organics dried (MgSO₄) and evaporated to give the title compound (6.21 g, 100%) as a yellow solid.

LC/MS Rt 3.06 min

### Description 42

### Methyl 5-{[5-chloro-2-(1-methylethyl)-1-benzofuran-7-yl]methyl}-2-furancarboxylate (D42)

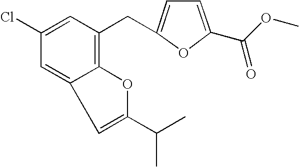

1,2-Dibromoethane (0.02 ml, 0.23 mmol) was added to a stirred suspension of zinc dust (227 mg, 3.48 mmol) in THF (10 ml) under argon and heated at 60° C. for three minutes then cooled to 35° C. and chlorotrimethylsilane (0.06 ml, 0.47 mmol) was added. The mixture was stirred at room temperature for thirty minutes and 7-(bromomethyl)-5-chloro-2-(1-methylethyl)-1-benzofuran (259 mg, 0.9 mmol) was added. After a further thirty minutes at room temperature methyl 5-bromo-2-furoate (185 mg, 0.9 mmol) and tetrakis(triphenylphosphine)palladium(0) (58 mg, 0.05 mmol) were added. The mixture was heated at 50° C. for six hours, cooled, diluted with ether/water and the organic phase, dried (magnesium sulphate), evaporated and purified by flash chromatography on silica gel eluting with ethyl acetate/hexane (3:47) to give the title compound as a colourless oil (55 mg).

LC/MS: Rt=3.91 min, [MH⁺] 333.2

### Description 43

### Ethyl 1-{[5-chloro-2-(1-methylethyl)-1-benzofuran-7-yl]methyl}-5-methyl-1H-pyrazole-3-carboxylate (D43)

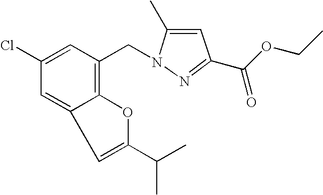

To a solution of ethyl 5-methyl-1H-pyrazole-3-carboxylate (385 mg) in dimethylformamide (10 ml) was added 7-(bromomethyl)-5-chloro-2-(1-methylethyl)-1-benzofuran (720 mg; may be prepared as described in D4) and potassium carbonate (380 mg) and the solution stirred at ambient temperature for 18 hours. It was concentrated in vacuo and water (20 ml) and ethyl acetate (30 ml) added. The organic layer was washed with brine (10 ml), dried (MgSO₄) and concentrated in vacuo. The residue was purified by Biotage® chromatography eluting with:

(a) 9:1 hexane:ethyl acetate to afford the regioisomer ethyl 1-{[5-chloro-2-(1-methylethyl)-1-benzofuran-7-yl]methyl}-3-methyl-1H-pyrazole-5-carboxylate (289 mg, 32%) as a pale buff oil.

LC/MS [MH+]=361/363, Rt=3.74 min.

(b) 4:1 hexane:ethyl acetate to afford the title compound (532 mg, 59%) as a white solid.

LC/MS [MH+]=361/363, Rt=4.09 min.

### Description 44

### Ethyl 1-{[5-bromo-2-(1-methylethyl)-1-benzofuran-7-yl]methyl}-5-methyl-1H-pyrazole-3-carboxylate (D44)

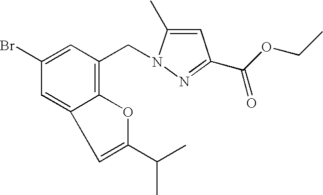

Ethyl 5-methyl-1H-pyrazole-3-carboxylate (1.58 g, 10.24 mmol), 7-(bromomethyl)-5-bromo-2-(1-methylethyl)-1-benzofuran (3.40 g, 10.24 mmol) and potassium carbonate (1.55 g, 11.21 mmol) were stirred in dimethylformamide (40 ml) at room temperature under argon for 18 hours. DMF was removed under reduced pressure (u.r.p.) and EtOAc (80 ml) and water (60 ml) added. The organic layer was separated, washed with brine (30 ml), dried over MgSO₄ and evaporated down under reduced pressure (u.r.p.). The residue was purified by chromatography (SP4 Biotage®) on silica gel using 5-40% EtOAc in hexane to give the title compound (2.52 g, 60.7%) as a pale buff solid.

LC/MS [M+1]=405, 407.

### Description 45

### Ethyl 1-[(5-chloro-2-propyl-1-benzofuran-7-yl)methyl]-5-methyl-1H-pyrazole-3-carboxylate (D45)

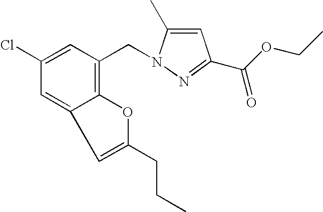

Ethyl 5-methyl-1H-pyrazole-3-carboxylate (911 mg, 5.911 mmol), 7-(bromomethyl)-5-chloro-2-propyl-1-benzofuran (1.70 g, 5.911 mmol) and potassium carbonate (899 mg, 6.505 mmol) were stirred in dimethylformamide (23 ml) at room temperature under argon for 8 hours. DMF was removed under reduced pressure (u.r.p.) and EtOAc (40 ml) and water (30 ml) added. The organic layer was separated, washed with brine (15 ml), dried over MgSO₄ and evaporated down under reduced pressure (u.r.p.). The residue was purified by chromatography (Biotage®) on silica gel using 9:1 hexane:EtOAc (to elute the regioisomer 695 mg, 32.6%) then 4:1 hexane:EtOAc to give the title compound (1.32 g, 62.0%) as a white solid.

LC/MS [M+1]=361, 363.

### Description 46

### Ethyl 1-[(5-chloro-2-cyclopropyl-1-benzofuran-7-yl)methyl]-5-methyl-1H-pyrazole-3-carboxylate (D46)

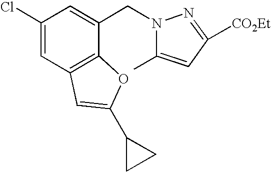

Solution of 5-chloro-7-(chloromethyl)-2-cyclopropyl-1-benzofuran (3.99 mmol) in dry DMF (16.0 ml) was stirred at room temperature under an atmosphere of argon. Ethyl-3-methyl-5-carboxylate (0.738 g, 4.79 mmol) and K₂CO₃ (0.827 g, 5.99 mmol) were added and the mixture stirred at room temperature for 1 hour. After this time more K₂CO₃ (0.827 g, 5.99 mmol) was added and the mixture stirred overnight at room temperature. Mixture was then heated to 80° C. for 3 hours. Solution was allowed to cool to room temperature and left stirring overnight. Mixture was diluted with EtOAc and organics washed with water (×3). Organics were dried over MgSO₄, filtered and concentrated under reduced pressure to give brown oil. The residue was chromatographed [SiO₂, Hexane EtOAc, 25-35%] to give the title compound (0.473 g).

LC/MS Rt=3.54 mins, [MH⁺] 359, 361.

### Description 47

### Ethyl 5-methyl-1-{[2-(1-methylethyl)-5-(methylsulfonyl)-1-benzofuran-7-yl]methyl}-1H-pyrazole-3-carboxylate (D47)

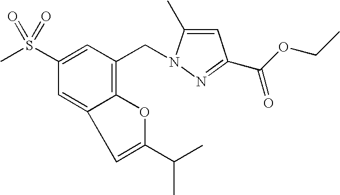

A mixture of [2-(1-methylethyl)-5-(methylsulfonyl)-1-benzofuran-7-yl]methyl methanesulfonate (295 mg, 0.83 mmol), K₂CO₃ (276 mg, 2 mmol) and ethyl 3-methylpyrazole-5-carboxylate (154 mg, 1 mmol) in DMF (4 ml) was stirred at room temperature for 20 hours then left to stand for 20 hours. Diluted with water & EtOAc (30 ml each) and the organic phase washed with water (3×15 ml), dried (MgSO₄), evaporated and purified by chromatography on silica gel (Biotage) with 1:2 EtOAc/hexane to give two products. The product that eluted first was identified as the regioisomeric analogue (125 mg), by NMR, as a white solid. The compound that eluted second was identified as the title compound (101 mg), by NMR and LC/MS, as a white solid.

LC/MS Rt 3.02 min, [MH+]=405.2.

### Description 48

### Ethyl 5-{[5-chloro-2-(1-methylethyl)-1-benzofuran-7-yl]methyl}-2-furancarboxylate (D48)

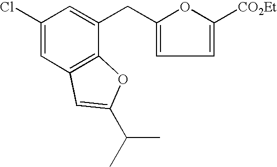

A mixture of ethyl 5-[(5-chloro-2-hydroxy-3-iodophenyl)methyl]-2-furancarboxylate (6.71 g, 16.5 mmol), triethylamine (2.244 g, 33 mmol), 3-methylbut-1-yne (3.333 g, 33 mmol), CuI (330 mg, 1.65 mmol) and (Ph₃P)₂PdCl₂ (1.138 g, 1.65 mmol) in DMF (50 ml) was stirred at room temperature under argon for 30 minutes then heated at 70° C. for 4 hours. Cooled, diluted with water (200 ml) and ether (150 ml) and the organic phase washed with water (3×50 ml) then dried (MgSO₄), evaporated and chromatographed on silica gel (Biotage®) with 6% EtOAc in hexane followed by 45:55 DCM-hexane to give the title compound (4.05 g) as a brownish oil. Left over the weekend in hexane (25 ml) but would not crystallise so evaporated (vacced off) and used as was.

LC/MS Rt 3.96 min, [MH+] 347.2—not pure but OK for next step.

The following compound was prepared in a similar manner.

| Description | Structure | Name | LC/MS data |
|---|---|---|---|
| D49 | 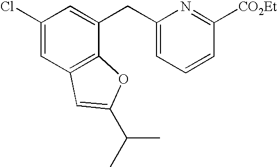 | Ethyl 6-{[5-chloro-2-(2-methylethyl)-1-benzofuran-7-yl]methyl}-2-pyridinecarboxylate | Rt = 3.74 min[M + H]⁺ 358.2 |

### Description 50

### Ethyl 1-[(5-chloro-2-cyclohexyl-1-benzofuran-7-yl)methyl]-5-methyl-1H-pyrazole-3-carboxylate

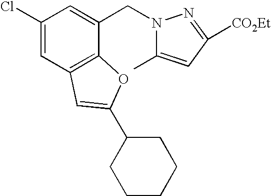

5-Chloro-2-cyclohexyl-7-(iodomethyl)-1-benzofuran (772 mg, 2.06 mmol) dissolved in dry DMF (8 ml). Pyrazole derivative (350 mg, 2.27 mmol) and K₂CO₃ (313 mg, 2.27 mmol) added. Reaction solution stirred at room temperature for 30 minutes. Sample removed for LC/MS analysis—starting material still present. Left at room temperature under an atmosphere of argon for 17 hours (overnight). Sample removed for LC/MS analysis—reaction complete. Reaction mixture diluted with EtOAc and washed with water (3×10 ml). Organics (yellow) dried over MgSO₄, filtered and concentrated to give a yellow oil. The residue was chromatographed [SiO₂, Hexane EtOAc, 0-15%] to give the title compound (366 mg, 44%.

LC/MS Rt=3.90 mins, [M+H] 401, 403.

### Description 51

### Ethyl 1-{[5-chloro-2-(1,1-dimethylethyl)-1-benzofuran-7-yl]methyl}-5-methyl-1H-pyrazole-3-carboxylate (D51)

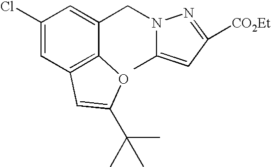

Prepared in a similar manner to ethyl 1-[(5-chloro-2-cyclohexyl-1-benzofuran-7-yl)methyl]-5-methyl-1H-pyrazole-3-carboxylate using 5-chloro-2-(1,1-dimethylethyl)-7-(iodomethyl)-1-benzofuran, except 1.5 equivalents of NaI were added.

LC/MS Rt=3.66 mins, [M+H] 375, 377.

### Description 52

### 2-(Trimethylsilyl)ethyl(1-{[5-chloro-2-(1-methylethyl)-1-benzofuran-7-yl]methyl}-5-methyl-1H-pyrazol-3-yl)carbamate (D52)

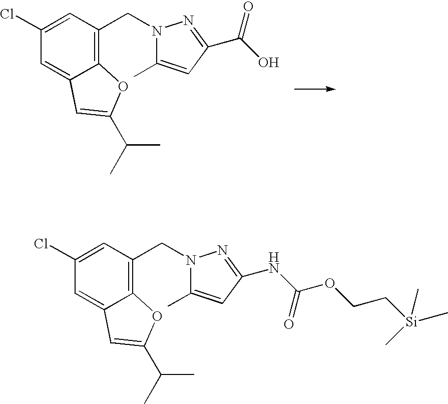

A slurry of 1-{[5-chloro-2-(1-methylethyl)-1-benzofuran-7-yl]methyl}-5-methyl-1H-pyrazole-3-carboxylic acid (0.500 g, 1.502 mmol) and diphenyl phosphoryl azide (0.36 ml, 1.653 mmol) in dry PhCH₃ (6 ml) was stirred at room temperature under an atmosphere of argon for 30 minutes. After this time, 2-(trimethylsilyl)ethanol (0.32 ml, 2.250 mmol) and triethylamine (0.25 ml, 1.803 mmol) were added. The reaction mixture was stirred at 90° C. under an atmosphere of argon for 17 hours. The reaction was monitored by LC/MS. The reaction mixture was allowed to cool and partitioned between EtOAc and NaHCO₃ (sat. aq solution). The organics were washed with water, dried over magnesium sulfate, filtered and concentrated under reduced pressure to give an orange oil. The residue was chromatographed [SiO₂, EtOAc:Hexane 0-40%] to give 2-(trimethylsilyl)ethyl (1-{[5-chloro-2-(1-methylethyl)-1-benzofuran-7-yl]methyl}-5-methyl-1H-pyrazol-3-yl)carbamate (0.448 g, 67%).

LC/MS Rt=4.03 min, [MH⁺] 448,450

### Description 53

### 2-(Trimethylsilyl)ethyl(1-{[5-cyano-2-(1-methylethyl)-1-benzofuran-7-yl]methyl}-5-methyl-1H-pyrazol-3-yl)carbamate (D53)

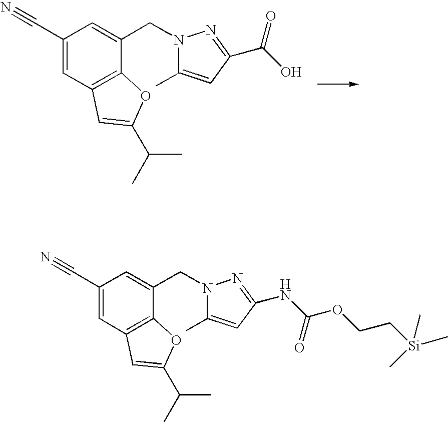

To a suspension of 1-{[5-cyano-2-(1-methylethyl)-1-benzofuran-7-yl]methyl}-5-methyl-1H-pyrazole-3-carboxylic acid (2.13 g) in dry toluene (30 ml) was added diphenylphosphoryl azide (1.56 ml) and the mixture stirred under argon at room temperature for thirty minutes. 2-Triethylsilyl ethanol (1.41 ml) and triethylamine (1.1 ml) were added and stirring continued for thirty minutes. The reaction mixture was then heated at 90° C. for four and a half hours. The reaction mixture was diluted with ethyl acetate (250 ml) and washed with saturated sodium bicarbonate (200 ml) and water (2×200 ml) then dried (MgSO₄) and evaporated to afford an orange solid. The solid was dissolved in dichloromethane and applied to a Biotage Si 40+M column and purified using the Biotage SP4® (gradient method) to afford the title compound as a white solid (1.815 g).

LC/MS Rt=3.88 min. Molecular ion observed [MH+] 439, consistent with molecular formula C₂₃H₃₀N₄O₃Si

### Description 54

### 1-{[5-Chloro-2-(1-methylethyl)-1-benzofuran-7-yl]methyl}-5-methyl-1H-pyrazol-3-amine (D54)

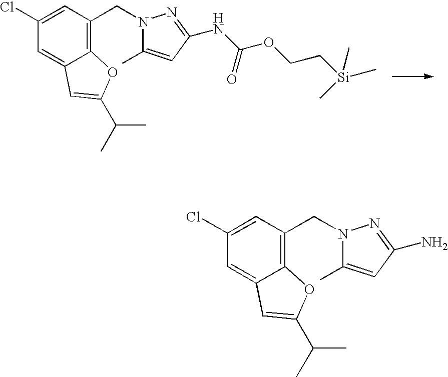

A solution of 2-(trimethylsilyl)ethyl (1-{[5-chloro-2-(1-methylethyl)-1-benzofuran-7-yl]methyl}-5-methyl-1H-pyrazol-3-yl)carbamate (0.448 g, 1.00 mmol) and tetrabutyl ammonium fluoride in THF (1M, 2 ml) in dry THF (2.3 ml) was stirred at 50° C. under an atmosphere of argon for 2 hours. The reaction was monitored by LC-MS. The reaction mixture was allowed to cool and concentrated under reduced pressure. The resulting residue was partitioned between EtOAc and water. The organics were washed with water (2×20 ml), dried over magnesium sulfate, filtered and concentrated under reduced pressure to give 1-{[5-chloro-2-(1-methylethyl)-1-benzofuran-7-yl]methyl}-5-methyl-1H-pyrazol-3-amine (0.304 g, 100%).

LC/MS Rt=2.76 min, [MH⁺] 304,306

### Description 55

### 7-[(3-Amino-5-methyl-1H-pyrazol-1-yl)methyl]-2-(1-methylethyl)-1-benzofuran-5-carbonitrile (D55)

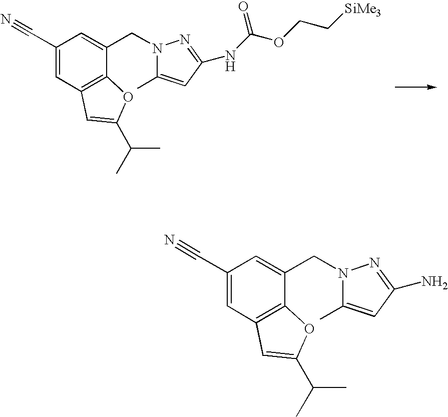

To a solution of 2-(trimethylsilyl)ethyl (1-{[5-cyano-2-(1-methylethyl)-1-benzofuran-7-yl]methyl}-5-methyl-1H-pyrazol-3-yl)carbamate (1.81 g) in dry tetrahydrofuran (10 ml) under argon was added tetrabutylammonium fluoride solution (1M in tetrahydrofuran, 8.3 ml) and the reaction mixture heated at 50° C. for one and three quarter hours. The mixture was cooled and the tetrahydrofuran evaporated. The residue was dissolved in ethyl acetate (30 ml) and washed with water (3×60 ml) then dried (MgSO₄) and evaporated to afford the title compound as a white solid (1.144 g).

LC/MS Rt=2.51 min. Molecular ion observed [MH+] 295, consistent with molecular formula C₁₇H₁₈N₄O

### Description 56

### 1-{[5-Chloro-2-(1-methylethyl)-1-benzofuran-7-yl]methyl}-N-[4-hydroxymethyl)phenyl]-5-methyl-1H-pyrazole-3-carboxamide (D56)

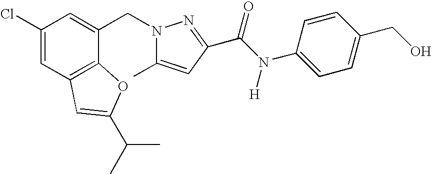

A solution of 1-{[5-chloro-2-(1-methylethyl)-1-benzofuran-7-yl]methyl}-5-methyl-1H-pyrazole-3-carboxylic acid (E3; 0.40 g, 1.20 mmol) in dry DMF (4 ml) was stirred at room temperature under an atmosphere of argon. EDAC (0.28 g, 1.44 mmol), HOBt (0.19 g, 1.44 mmol) and 4-aminobenzyl alcohol (0.18 g, 1.44 mmol) were added to the stirred solution. The solution was stirred at room temperature under an atmosphere of argon for 72 hours (over the weekend). The reaction was monitored by LC-MS. Reaction solution was diluted with EtOAc. Organics were washed with an aqueous solution of potassium carbonate. Organics were separated and washed with further water (3×50 ml). Organics were dried over magnesium sulfate, filtered and concentrated under reduced pressure to give a brown solid (486 mg). The crude brown solid was washed with methanol. The resulting cream precipitate was filtered off to give the title compound (350 mg, 66%).

LC/MS Rt=3.33 min [MH⁺]: 438, 440

### Description 57

### 1-{[5-Chloro-2-(1-methylethyl)-1-benzofuran-7-yl]methyl}-N-[4-hydroxymethyl)phenyl]-5-methyl-1H-pyrazole-3-carboxamide (D57)

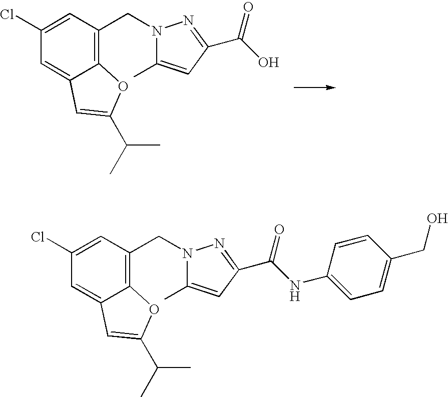

A mixture of 1-{[5-chloro-2-(1-methylethyl)-1-benzofuran-7-yl]methyl}-5-methyl-1H-pyrazole-3-carboxylic acid (0.650 g, 1.95 mmol), EDAC (0.449 g, 2.34 mmol), HOBt (0.317 g, 2.34 mmol) and 4-aminobenzylalcohol (0.289 g, 2.34 mmol) in dry DMF (6.5 ml) was stirred at room temperature under an atmosphere of argon for 17 hours (overnight). The reaction was monitored by LC-MS. The reaction mixture partitioned between EtOAc and K₂CO₃ (sat. aq solution). The organics were washed with water (3×50 ml), dried over magnesium sulfate, filtered and concentrated under reduced pressure to give a cream solid, 1-{[5-chloro-2-(1-methylethyl)-1-benzofuran-7-yl]methyl}-N-[4-(hydroxymethyl)phenyl]-5-methyl-1H-pyrazole-3-carboxamide (0.794 g, 93%).

LC/MS Rt=3.30 min, [MH⁺] 438,440

### Description 58

### N-(1-{[5-chloro-2-(1-methylethyl)-1-benzofuran-7-yl]methyl}-5-methyl-1H-pyrazol-3-yl)-4-(hydroxymethyl)benzamide (D58)

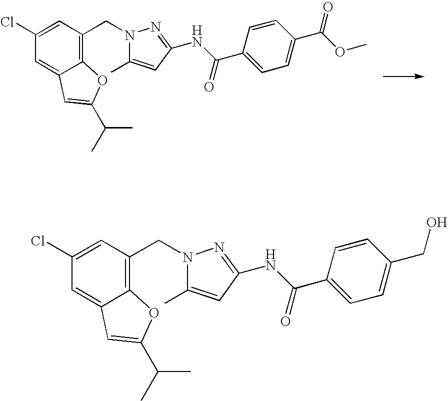

A solution of methyl 4-{[(1-{[5-chloro-2-(1-methylethyl)-1-benzofuran-7-yl]methyl}-5-methyl-1H-pyrazol-3-yl)amino]carbonyl}benzoate (0.460 g, 0.989 mmol) and lithium aluminium hydride in diethyl ether (1M, 2 ml) in dry THF (6.6 ml) was stirred at 0° C. under an atmosphere of argon for 2 hours. The reaction was monitored by LC-MS. The reaction mixture was quenched by the slow addition of water (50 ml) and stirred, warming to room temperature over 10 minutes. After this time, the reaction mixture was diluted with water and the organics were extracted using EtOAc (×3). The combined organics were dried over magnesium sulfate, filtered and concentrated under reduced pressure to give a white solid, N-(1-{[5-chloro-2-(1-methylethyl)-1-benzofuran-7-yl]methyl}-5-methyl-1H-pyrazol-3-yl)-4-(hydroxymethyl)benzamide (0.350 g, 81%)

LC/MS Rt=3.20 min, [MH⁺] 438, 440

### Description 59

### 1-{[5-Chloro-2-(1-methylethyl)-1-benzofuran-7-yl]methyl}-N-(4-formylphenyl)-5-methyl-1H-pyrazole-3-carboxamide (D59)

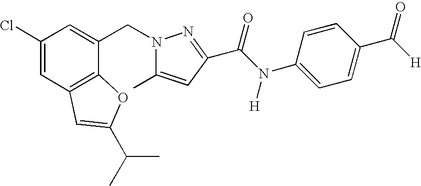

A solution of 1-{[5-chloro-2-(1-methylethyl)-1-benzofuran-7-yl]methyl}-N-[4-(hydroxymethyl)phenyl]-5-methyl-1H-pyrazole-3-carboxamide (D7; 0.34 g, 0.78 mmol) in dry DCM (4 ml) was stirred at 0° C. under an atmosphere of argon. Dess-Martin periodinane (0.33 g, 0.78 mmol) was added to the stirred solution. The reaction mixture was allowed to warm from 0° C. to room temperature while stirring over a period of 2 hours. The reaction was monitored by LC-MS. The reaction mixture was cooled to 0° C. and quenched by the slow addition of sodium bicarbonate solution (20 ml) and sodium thiosulfate solution (20 ml). The resulting solution was stirred at 0° C. under an atmosphere of argon for 1 hour. The organics were extracted with DCM (×3). Brine was added to aid the separation. The dark coloured interface was retained in the aqueous layer. The combined organics were dried over magnesium sulfate, filtered and concentrated under reduced pressure to give the title compound as a pale brown solid (310 mg, 92%).

LC/MS Rt=3.72 min [MH⁺]: 436, 438

### Description 60

### 1-{[5-Bromo-2-(1-methylethyl)-1-benzofuran-7-yl]methyl}-N-(4-formylphenyl)-5-methyl-1H-pyrazole-3-carboxamide (D60)

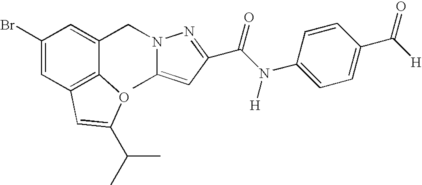

To a stirring solution of 1-{[5-bromo-2-(1-methylethyl)-1-benzofuran-7-yl]methyl}-N-[4-(hydroxymethyl)phenyl]-5-methyl-1H-pyrazole-3-carboxamide (714 mg, 1.48 mmol) in dry DCM (10 ml) was added Dess-Martin periodinane (DMP) (628 mg, 1.48 mmol) and the mixture stirred at room temp. under argon for 3 hours. The mixture was washed with saturated NaHCO₃ solution (10 ml) and sodium thiosulfate solution and the aqueous layer extracted with DCM (2×5 ml). The combined DCM layers were washed with brine (7.5 ml), dried over magnesium sulphate and evaporated down under reduced pressure (u.r.p.). The residue was purified by chromatography on silica gel (Biotage SP4) using 5-40% EtOAc in hexane to give the title compound as a pale buff solid (139 mg). Further chromatography using 9:1 DCM:methanol gave further title compound (544 mg) as a pale buff yellow solid. Total yield 683 mg (96.1%).

LC/MS [M+1]: 480, 482

### Description 61

### 5-{[5-Chloro-2-(1-methylethyl)-1-benzofuran-7-yl]methyl}-N-(4-formylphenyl)-2-furancarboxamide (D61)

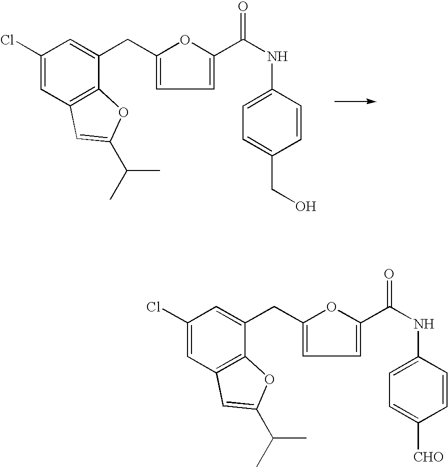

Dess-Martin periodinane (848 mg, 2 mmol) was added to a stirred solution of 5-{[5-chloro-2-(1-methylethyl)-1-benzofuran-7-yl]methyl}-N-[4-(hydroxymethyl)phenyl]-2-furancarboxamide (560 mg, 1.32 mmol) in dichloromethane (15 ml) under argon and stirred for 2 hours. The resulting solution was washed with a solution of sodium thiosulphate pentahydrate (5 g) in saturated sodium bicarbonate (50 ml), dried (magnesium sulphate), evaporated and purified by flash chromatography on a Biotage column eluting with 1:3 ethyl acetate/hexane to give the title compound as colourless gum (494 mg).

LC/MS: Rt=3.80 min, [M+H]⁺ 422.1, 424.1

### Description 62

### N-(1-{[5-chloro-2-(1-methylethyl)-1-benzofuran-7-yl]methyl}-5-methyl-1H-pyrazol-3-yl)-4-formylbenzamide (D62)

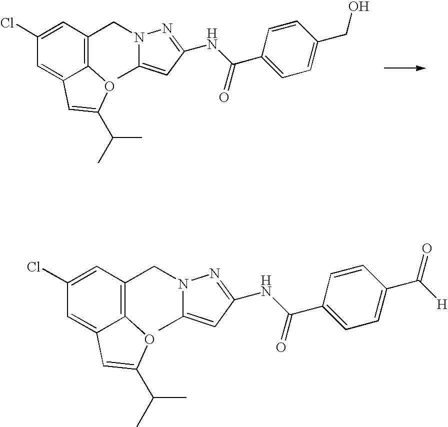

To a solution of N-(1-{[5-chloro-2-(1-methylethyl)-1-benzofuran-7-yl]methyl}-5-methyl-1H-pyrazol-3-yl)-4-(hydroxymethyl)benzamide (0.350 g, 0.800 mmol) in dry DCM (4 ml) at 0° C. under an atmosphere of argon, Dess-Martin periodinane (0.340 g, 0.800 mmol) was added. The reaction mixture was stirred for 30 minutes, warming from 0° C. to room temperature. The reaction mixture was stirred at room temperature for 2 hours. The reaction was monitored by LC-MS. The reaction mixture was cooled to 0° C. and quenched using NaHCO₃ (20 ml, sat. aq soln) and Na₂S₂O₃ (20 ml, 10% aq. soln). The organics were extracted using DCM. The combined organics were dried over magnesium sulfate, filtered and concentrated under reduced pressure to give a cream solid, N-(1-{[5-chloro-2-(1-methylethyl)-1-benzofuran-7-yl]methyl}-5-methyl-1H-pyrazol-3-yl)-4-formylbenzamide (0.350 g, 100%).

LC/MS Rt=3.46 min, [MH⁺] 436,438

### Description 63

### Methyl 4-{[(1-{[5-chloro-2-(1-methylethyl)-1-benzofuran-7-yl]methyl}-5-methyl-1H-pyrazol-3-yl)amino]carbonyl}benzoate (D63)

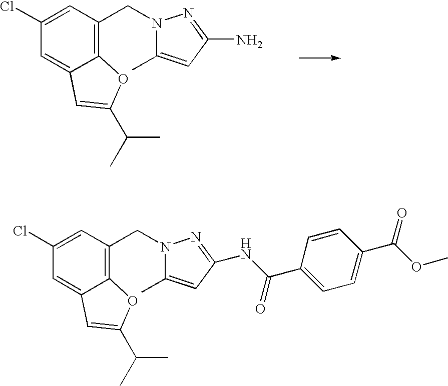

A mixture of 1-{[5-chloro-2-(1-methylethyl)-1-benzofuran-7-yl]methyl}-5-methyl-1H-pyrazol-3-amine (0.334 g, 1.10 mmol), EDAC (0.254 g, 1.32 mmol), HOBt (0.179 g, 1.32 mmol) and 4-[(methyloxy)carbonyl]benzoic acid (0.238 g, 1.32 mmol) in dry DMF (4 ml) was stirred at room temperature under an atmosphere of argon for 17 hours (overnight). The reaction was monitored by LC-MS. The reaction mixture was partitioned between EtOAc and NaHCO₃ (sat. aq solution). The organics were washed with water (3×20 ml), dried over magnesium sulfate, filtered and concentrated under reduced pressure to give a colourless oil. The residue was chromatographed [SiO₂, EtOAc:Hexane 10-50%] to give methyl 4-{[(1-{[5-chloro-2-(1-methylethyl)-1-benzofuran-7-yl]methyl}-5-methyl-1H-pyrazol-3-yl)amino]carbonyl}benzoate (0.465 g, 91%)

LC/MS Rt=3.60 min, [MH⁺] 466,468

### Description 64

### 1-{[5-chloro-2-(1-methylethyl)-1-benzofuran-7-yl]methyl}-5-methyl-1H-pyrazole-3-carbonyl Chloride (D64)

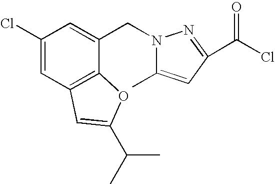

Solution of 1-{[5-chloro-2-(1-methylethyl)-1-benzofuran-7-yl]methyl}-5-methyl-1H-pyrazole-3-carboxylic acid (0.450 g, 1.36 mmol) in dry DCM (5.5 ml) was stirred at room temperature under an atmosphere of argon. SOCl₂ (0.294 ml, 4.06 mmol) was added and the solution heated to 45-50° C. for 1½ hours. After this time LC/MS showed no starting material. Solvent was removed under reduced pressure to give a yellow coloured oil. Residue was used directly in the next step.

The following compounds were prepared in a similar manner using the appropriate acid, or sodium salt of the acid, and thionyl chloride at temperature of 40-60° C. for up to 2½ hours.

| Description | Structure | Name |
|---|---|---|
| D65 | 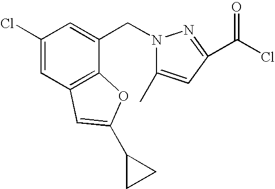 | 1-[(5-Chloro-2-cyclopropyl-1-benzofuran-7-yl)methyl]-5-methyl-1H-pyrazole-3-carbonyl chloride |
| D66 | 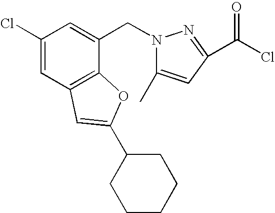 | 1-[(5-Chloro-2-cyclohexyl-1-benzofuran-7-yl)methyl]-5-methyl-1H-pyrazole-3-carbonyl chloride |

### Description 67

### 1,1-Dimethylethyl 4-{[(1-{[5-chloro-2-(1-methylethyl)-1-benzofuran-7-yl]methyl}-5-methyl-1H-pyrazol-3-yl)carbonyl]amino}-1-piperidinecarboxylate (D67)

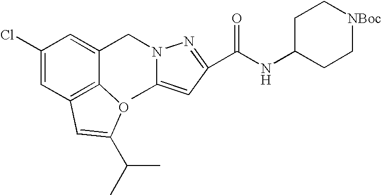

Solution of 1-{[5-chloro-2-(1-methylethyl)-1-benzofuran-7-yl]methyl}-5-methyl-1H-pyrazole-3-carbonyl chloride (0.68 mmol) in dry DCM (2.7 ml) was stirred at room temperature under an atmosphere of argon. Et₃N (0.114 ml, 0.82 mmol) was added to the solution. 1,1-dimethylethyl 4-amino-1-piperidinecarboxylate (0.164 g, 0.82 mmol) was added to the mixture and stirring continued for 2 hours. After this time, solution was diluted with Et₂O and washed with water (×3). Organics were dried over MgSO₄, filtered and concentrated under reduced pressure to give a pale yellow solid. Residue was chromatographed [SiO₂ Hexane/EtOAc, 50-100%] to give pure product (0.286 g).

LC/MS Rt=3.73 mins [MH⁺] 515.

The following compounds were prepared in a similar manner to above using the appropriate amine and the appropriate acid chloride, with reactions stirring at room temperature for 2-18 hours. The compounds were purified by chromatography or using the MDAP.

| | | | LC/MS |
|---|---|---|---|
| Description | Structure | Name | Data |
| D68 | 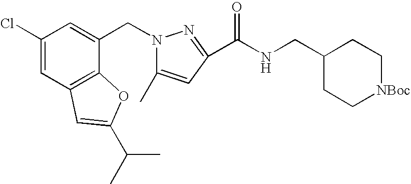 | 1,1-Dimethylethyl 4-({[(1-{[5-chloro-2-(1-methylethyl)-1-benzofuran-7-yl]methyl}-5-methyl-1H-pyrazol-3-yl)carbonyl]amino}methyl)-1-piperidinecarboxylate | Rt = 3.78mins [MH⁺]529, 531 |

### Example 1

### 5-{[5-Chloro-2-(1-methylethyl)-1-benzofuran-7-yl]methyl}-2-furancarboxylic Acid (E1)

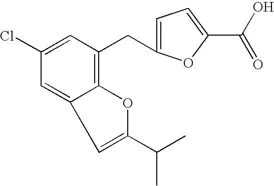

2M Sodium hydroxide (1 ml, 2 mmol) was added to a solution of methyl 5-{[5-chloro-2-(1-methylethyl)-1-benzofuran-7-yl]methyl}-2-furancarboxylate (50 mg, 0.15 mmol) in methanol (5 ml) and heated to reflux then left to cool for two hours. After evaporation the residue was dissolved in ethyl acetate/water/brine and the organic phase separated and washed with 2M hydrochloric acid then dried (magnesium sulphate), evaporated and triturated with ether/hexane (1:4) to give the title compound as a white solid (35 mg).

LC/MS: Rt=3.43 min, [MH⁺] 319.2

### Example 2

### 2-(5-{[5-Chloro-2-(1-methylethyl)-1-benzofuran-7-yl]methyl}-2-furanyl)-1H-benzimidazole Hydrochloride (E2)

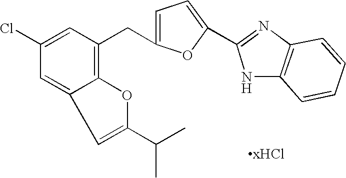

### (a) N-(2-Aminophenyl)-5-{[5-chloro-2-(1-methylethyl)-1-benzofuran-7-yl]methyl}-2-furancarboxamide Hydrochloride

A mixture of 1,2-phenylenediamine (32 mg, 0.3 mmol), 5-{[5-chloro-2-(1-methylethyl)-1-benzofuran-7-yl]methyl}-2-furancarboxylic acid (83 mg, 0.26 mmol), hydroxybenzotriazole (46 mg, 0.3 mmol) and EDAC (64 mg, 0.33 mmol) in dichloromethane (5 ml) was stirred at room temperature for four hours. The resulting solution was diluted with ether/water, washed with saturated sodium bicarbonate, dried (magnesium sulphate), evaporated and the residue purified by chromatography on silica gel eluting with ethyl acetate/hexane (1:2) to give the title compound as a gum which crystallised (76 mg).

LC/MS: Rt=3.56 min, [MH⁺] 409.1, 411.1

### (b) 2-(5-{[5-Chloro-2-(1-methylethyl)-1-benzofuran-7-yl]methyl}-2-furanyl)-1H-benzimidazole Hydrochloride

A solution of N-(2-aminophenyl)-5-{[5-chloro-2-(1-methylethyl)-1-benzofuran-7-yl]methyl}-2-furancarboxamide hydrochloride (70 mg, 0.17 mmol) in acetic acid (4 ml) was stirred and refluxed for one hour then cooled and evaporated to dryness. The residue was dissolved in ethyl acetate, washed with saturated sodium bicarbonate solution, dried (magnesium sulphate), and evaporated. The residue was triturated with ether/hexane (1:1), filtered off, dissolved in dichloromethane/methanol and 1M hydrogen chloride in ether (1 ml) was added. The solution was evaporated to dryness and the residue triturated with ether and the solvent removed by pipette to leave the title compound as a white solid (53 mg).

LC/MS: Rt=3.09 min, [MH⁺] 391.2, 393.2

### Example 3

### 1-{[5-Chloro-2-(1-methylethyl)-1-benzofuran-7-yl]methyl}-5-methyl-1H-pyrazole-3-carboxylic Acid (E3)

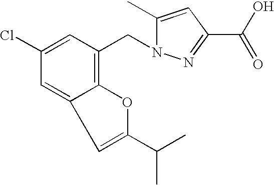

A solution of ethyl 1-{[5-chloro-2-(1-methylethyl)-1-benzofuran-7-yl]methyl}-5-methyl-1H-pyrazole-3-carboxylate (540 mg; may be prepared as described in D6) in 2M sodium hydroxide solution (3 ml) and ethanol (12 ml) was stirred at ambient temperature for 1.5 hours. It was concentrated in vacuo and water (20 ml) added. The mixture was treated to pH 1 with 5M hydrochloric acid and the precipitated solid filtered, washed with water and dried in vacuo at 50° to afford the title compound (477 mg, 96%) as a white solid.

LC/MS [MH+]=333/335, Rt=3.14 min.

### Example 4

### 4-Piperidinylmethyl (1-{[5-chloro-2-(1-methylethyl)-1-benzofuran-7-yl]methyl}-5-methyl-1H-pyrazol-3-yl)carbamate Hydrochloride (E4)

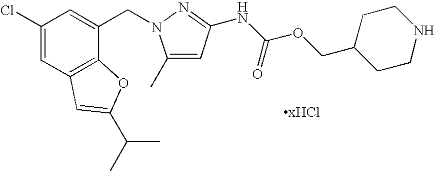

### (a). 1,1-Dimethylethyl 4-[({[(1-{[5-chloro-2-(1-methylethyl)-1-benzofuran-7-yl]methyl}-5-methyl-1H-pyrazol-3-yl)amino]carbonyl}oxy)methyl]-1-piperidinecarboxylate

To a solution of 1-{[5-chloro-2-(1-methylethyl)-1-benzofuran-7-yl]methyl}-5-methyl-1H-pyrazole-3-carboxylic acid (80 mg) in toluene (1 ml) was added triethylamine (40 μl) and diphenylphosphoryl azide (57 μl) and the solution stirred at 80° C. for 15 minutes. 1,1-dimethylethyl 4-(hydroxymethyl)-1-piperidinecarboxylate (129 mg) was added and the solution stirred at 80° C. for 3 hours. It was concentrated in vacuo and the residue was purified by mass-directed autopreparation to afford the title compound (68 mg, 52%) as a white solid.

LC/MS [MH+]=545/547, Rt=4.04 min.

### (b). 4-Piperidinylmethyl (1-{[5-chloro-2-(1-methylethyl)-1-benzofuran-7-yl]methyl}-5-methyl-1H-pyrazol-3-yl)carbamate Hydrochloride

A solution of 1,1-dimethylethyl 4-[({[(1-{[5-chloro-2-(1-methylethyl)-1-benzofuran-7-yl]methyl}-5-methyl-1H-pyrazol-3-yl)amino]carbonyl}oxy)-methyl]-1-piperidinecarboxylate (68 mg) in 4M hydrogen chloride in dioxan (0.8 ml) was stirred at ambient temperature for 1.5 hours. It was concentrated in vacuo and the residue washed with ether, filtered and dried in vacuo at 50° C. to afford the title compound (46 mg, 76%) as a white solid.

LC/MS [MH+]=445/447, Rt=2.03 min.

### Example 5

### 2-(1-{[5-Chloro-2-(1-methylethyl)-1-benzofuran-7-yl]methyl}-5-methyl-1H-pyrazol-3-yl)-1H-benzimidazole Hydrochloride (E5)

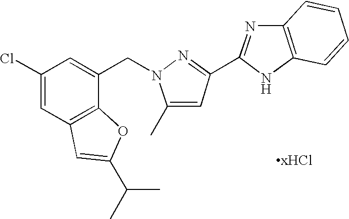

### (a). N-(2-Aminophenyl)-1-{[5-chloro-2-(1-methylethyl)-1-benzofuran-7-yl]methyl}-5-methyl-1H-pyrazole-3-carboxamide

A solution of 1-{[5-chloro-2-(1-methylethyl)-1-benzofuran-7-yl]methyl}-5-methyl-1H-pyrazole-3-carboxylic acid (83 mg), 1,2-phenylenediamine (32 mg), N-(3-dimethylaminopropyl)-N′-ethylcarbodiimide hydrochloride (64 mg) and 1-hydroxybenzotriazole hydrate (46 mg) in dichloromethane (5 ml) was stirred at ambient temperature for 4 hours. Ether (15 ml) and water (15 ml) were added and the organic layer separated and washed with saturated sodium bicarbonate solution (15 ml), dried (MgSO₄) and concentrated in vacuo. The residue was purified by Biotage chromatography eluting with 2:1 hexane:ethyl acetate to afford the title compound (86 mg, 82%) as a colourless gum.

LC/MS [MH+]=423/425, Rt=3.47 min.

### (b). 2-(1-{[5-Chloro-2-(1-methylethyl)-1-benzofuran-7-yl]methyl}-5-methyl-1H-pyrazol-3-yl)-1H-benzimidazole Hydrochloride

A solution of N-(2-aminophenyl)-1-{[5-chloro-2-(1-methylethyl)-1-benzofuran-7-yl]methyl}-5-methyl-1H-pyrazole-3-carboxamide (86 mg) in glacial acetic acid (4.5 ml) was stirred under reflux for 1 hour. After cooling, it was concentrated in vacuo and the residue dissolved in ethyl acetate (5 ml) and the solution washed with saturated sodium bicarbonate solution (5 ml), dried (MgSO₄) and concentrated in vacuo. The residue was purified by mass-directed autopreparation and the residue dissolved in a few mls of ethanol, 2M hydrogen chloride in ether (0.5 ml) added and solvent removed in vacuo. The residue was washed with ether, filtered and dried in vacuo at 50° to afford the title compound (56 mg, 62%) as a white solid.

LC/MS [MH+]=405/407, Rt=2.52 min.

### Example 6

### 1-{[5-Chloro-2-(1-methylethyl)-1-benzofuran-7-yl]methyl}-5-methyl-N-1-piperidinyl-1H-pyrazole-3-carboxamide (E6)

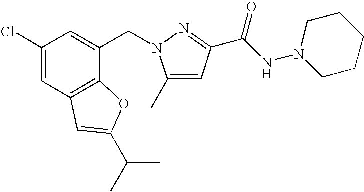

### (a). 1-{[5-Chloro-2-(1-methylethyl)-1-benzofuran-7-yl]methyl}-5-methyl-1H-pyrazole-3-carbonyl Chloride

A solution of 1-{[5-chloro-2-(1-methylethyl)-1-benzofuran-7-yl]methyl}-5-methyl-1H-pyrazole-3-carboxylic acid (166 mg) and thionyl chloride (182 μl) in dichloromethane (1.5 ml) was stirred under reflux for 4.5 hours. It was concentrated in vacuo and traces of thionyl chloride removed by azeotroping with toluene to afford the title compound (176 mg) as a colourless oil.

### (b). 1-{[5-Chloro-2-(1-methylethyl)-1-benzofuran-7-yl]methyl}-5-methyl-N-1-piperidinyl-1H-pyrazole-3-carboxamide

To an ice-cooled solution of 1-{[5-chloro-2-(1-methylethyl)-1-benzofuran-7-yl]methyl}-5-methyl-1H-pyrazole-3-carbonyl chloride (88 mg) in dichloromethane (1.2 ml) was added triethylamine (42 μl) and a solution of 1-aminopiperidine (33 μl) in dichloromethane (0.5 ml). The solution was stirred at 0° C. for 15 minutes then at ambient temperature for 30 minutes. It was concentrated in vacuo, ethyl acetate (5 ml) added and the solution washed with water (3 ml) and saturated sodium bicarbonate solution (3 ml), dried (MgSO₄) and concentrated in vacuo. The residue was purified by mass-directed autopreparation to afford the title compound (41 mg, 40%) as a white solid.

LC/MS [MH+]=415/417, Rt=3.38 min.

### Example 7

### 1-{[5-Chloro-2-(1-methylethyl)-1-benzofuran-7-yl]methyl}-5-methyl-N-4-morpholinyl-1H-pyrazole-3-carboxamide (E7)

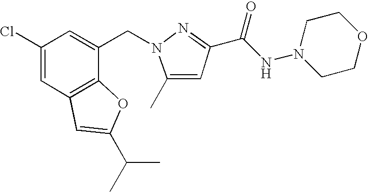

### (a) 1-{[5-Chloro-2-(1-methylethyl)-1-benzofuran-7-yl]methyl}-5-methyl-1H-pyrazole-3-carbonyl Chloride

A solution of 1-{[5-chloro-2-(1-methylethyl)-1-benzofuran-7-yl]methyl}-5-methyl-1H-pyrazole-3-carboxylic acid (166 mg) and thionyl chloride (182 μl) in dichloromethane (1.5 ml) was stirred under reflux for 4.5 hours. It was concentrated in vacuo and traces of thionyl chloride removed by azeotroping with toluene to afford the title compound (176 mg) as a colourless oil.

### (b) 1-{[5-Chloro-2-(1-methylethyl)-1-benzofuran-7-yl]methyl}-5-methyl-N-4-morpholinyl-1H-pyrazole-3-carboxamide

To an ice-cooled solution of 1-{[5-chloro-2-(1-methylethyl)-1-benzofuran-7-yl]methyl}-5-methyl-1H-pyrazole-3-carbonyl chloride (88 mg) in dichloromethane (1.2 ml) was added triethylamine (42 μl) and a solution of 4-aminomorpholine (29 μl) in dichloromethane (0.5 ml). The solution was stirred at 0° C. for 15 minutes then at ambient temperature for 30 minutes. It was concentrated in vacuo, ethyl acetate (5 ml) added and the solution washed with water (3 ml) and saturated sodium bicarbonate solution (3 ml), dried (MgSO₄) and concentrated in vacuo. The residue was purified by mass-directed autopreparation to afford the title compound (69 mg, 66%) as a white solid.

LC/MS [MH⁺]=417/419, Rt=2.99 min.

### Example 8

### 5-{[5-Chloro-2-(1-methylethyl)-1-benzofuran-7-yl]methyl}-N-(2,6-difluorophenyl)-2-furancarboxamide (E8)

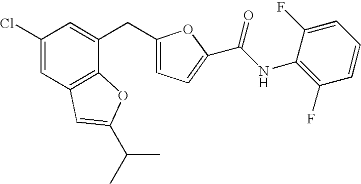

### (a). 5-{[5-Chloro-2-(1-methylethyl)-1-benzofuran-7-yl]methyl}-2-furan-carbonyl Chloride

To a solution of 5-{[5-chloro-2-(1-methylethyl)-1-benzofuran-7-yl]methyl}-2-furancarboxylic acid in dichloromethane (1 ml) and dimethylformamide (1 drop) was added oxalyl chloride (90 μl), and the solution stirred at ambient temperature for 1.5 hours. It was concentrated in vacuo and traces of oxalyl chloride removed by azeotroping with toluene to afford the title compound (58 mg) as a colourless oil.

### (b). 5-{[5-Chloro-2-(1-methylethyl)-1-benzofuran-7-yl]methyl}-N-(2,6-difluorophenyl)-2-furancarboxamide

To a solution of 2,6-difluoroaniline (29 μl) in dichloromethane (0.5 ml) and pyridine (0.1 ml) was added dropwise a solution of 5-{[5-chloro-2-(1-methylethyl)-1-benzofuran-7-yl]methyl}-2-furan-carbonyl chloride (58 mg) in dichloromethane (1 ml) and the mixture stirred at ambient temperature for 1 hour. It was washed with 2M hydrochloric acid (1 ml), dried (MgSO₄) and concentrated in vacuo. The residue was purified by mass-directed autopreparation to afford the title compound (38 mg, 51%) as a white solid.

LC/MS [MH+]=430/432, Rt=3.79 min.

### Example 9

### [2-(5-{[5-Chloro-2-(1-methylethyl)-1-benzofuran-7-yl]methyl}-2-furanyl)-1H-benzimidazol-5-yl]methanol Hydrochloride (E9)

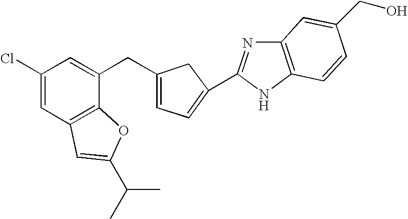

### (a). Methyl 4-amino-3-{[(5-{[5-chloro-2-(1-methylethyl)-1-benzofuran-7-yl]methyl}-2-furanyl)carbonyl]amino}benzoate

A mixture of 5-{[5-chloro-2-(1-methylethyl)-1-benzofuran-7-yl]methyl}-2-furancarboxylic acid (722 mg), methyl 3,4-diaminobenzoate (452 mg), N-(3-dimethylaminopropyl)-N′-ethylcarbodiimide hydrochloride (572 mg) and 1-hydroxybenzotriazole hydrate (416 mg) and dichloromethane (50 ml) was stirred at ambient temperature for 72 hours. Ethyl acetate (150 ml) and water (150 ml) were added and the organic layer separated and washed with saturated sodium bicarbonate solution (150 ml), dried (MgSO₄) and concentrated in vacuo. The residue was purified by Biotage® chromatography eluting with 2:1 hexane:ethyl acetate to afford the title compound (797 mg, 75%) as a pale buff solid.

LC/MS [MH+]=467/469, Rt=3.53 min.

### (b). Methyl 2-(5-{[5-chloro-2-(1-methylethyl)-1-benzofuran-7-yl]methyl}-2-furanyl)-1H-benzimidazole-5-carboxylate

A solution of methyl 4-amino-3-{[(5-{[5-chloro-2-(1-methylethyl)-1-benzofuran-7-yl]methyl}-2-furanyl)carbonyl]amino}benzoate (797 mg) in glacial acetic acid (40 ml) was stirred under reflux for 3 hours. After cooling, it was concentrated in vacuo and the residue dissolved in ethyl acetate (25 ml) and the solution washed with saturated sodium bicarbonate solution (15 ml), dried (MgSO₄) and concentrated in vacuo. The residue was triturated with hexane and dried in vacuo at 50° to afford the title compound (761 mg, 99%) as a pale buff solid.

LC/MS [MH+]=449/451, Rt=3.63 min.

### (c). [2-(5-{[5-Chloro-2-(1-methylethyl)-1-benzofuran-7-yl]methyl}-2-furanyl)-1H-benzimidazol-5-yl]methanol

To a solution of methyl 2-(5-{[5-chloro-2-(1-methylethyl)-1-benzofuran-7-yl]methyl}-2-furanyl)-1H-benzimidazole-5-carboxylate (761 mg) in tetrahydrofuran (20 ml) was added dropwise with ice-cooling a 1M solution of lithium aluminium hydride in tetrahydrofuran (3.2 ml) and the mixture stirred at ambient temperature for 2 hours. The mixture was quenched with a few mls of methanol and water, and ethyl acetate (30 ml) and 2M sodium hydroxide solution (25 ml) added. The organic layer was separated and washed with water (20 ml), dried (MgSO₄) and concentrated in vacuo. The residue was washed with hexane and dried in vacuo to afford the title compound (695 mg, 97%) as a pale buff solid.

LC/MS [MH+]=421/423, Rt=2.58 min.

### Example 10

### {[2-(5-{[5-Chloro-2-(1-methylethyl)-1-benzofuran-7-yl]methyl}-2-furanyl)-1H-benzimidazol-5-yl]methyl}methylamine Hydrochloride (E10)

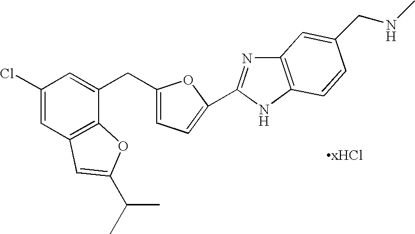

### (a). 2-(5-{[5-Chloro-2-(1-methylethyl)-1-benzofuran-7-yl]methyl}-2-furanyl)-1H-benzimidazole-5-carbaldehyde

To a solution of [2-(5-{[5-chloro-2-(1-methylethyl)-1-benzofuran-7-yl]methyl}-2-furanyl)-1H-benzimidazol-5-yl]methanol (655 mg) in dichloromethane (8 ml) was added under an argon atmosphere Dess-Martin periodinane (660 mg) and the solution stirred at ambient temperature for 1.25 hours. It was washed with saturated sodium bicarbonate solution (15 ml) and the aqueous layer extracted with dichloromethane (2×5 ml). The combined organic layers were washed with brine (8 ml), dried (MgSO₄) and concentrated in vacuo. The residue was purified by Biotage® chromatography eluting with 2:1 hexane:ethyl acetate to afford the title compound (512 mg, 79%) as a pale buff solid.

LC/MS [MH+]=419/421, Rt=3.51 min.

### (b). 1-[2-(5-{[5-chloro-2-(1-methylethyl)-1-benzofuran-7-yl]methyl}-2-furanyl)-1H-benzimidazol-5-yl]-N-methylmethanamine Hydrochloride

To a solution of 2-(5-{[5-chloro-2-(1-methylethyl)-1-benzofuran-7-yl]methyl}-2-furanyl)-1H-benzimidazole-5-carbaldehyde (64 mg) in tetrahydrofuran (2.5 ml) was added 33% methylamine in ethanol (78 μl) and the solution stirred at ambient temperature for 15 minutes. Sodium triacetoxyborohydride (130 mg) was added and stirring continued for 3 hours. Ethyl acetate (10 ml) and brine (10 ml) were added and the organic layer separated, dried (MgSO₄) and concentrated in vacuo. The residue was purified by mass-directed autopreparation and the residue dissolved in a few mls of ethanol, 2M hydrogen chloride in ether (0.5 ml) added and solvent removed in vacuo. The residue was washed with ether, filtered and dried in vacuo at 50° to afford the title compound (7.0 mg, 9%) as a pale buff solid.

LC/MS [MH+]=434/436, Rt=2.25 min.

### Example 11

### 1-[2-(5-{[5-Chloro-2-(1-methylethyl)-1-benzofuran-7-yl]methyl}-2-furanyl)-1H-benzimidazol-5-yl]-N,N-dimethylmethanamine Hydrochloride (E11)

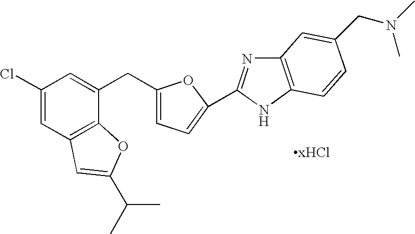

In a manner similar to E10, Step (e), 2-(5-{[5-chloro-2-(1-methylethyl)-1-benzofuran-7-yl]methyl}-2-furanyl)-1H-benzimidazole-5-carbaldehyde (130 mg), 33% dimethylamine in ethanol (110 μl) and sodium triacetoxyborohydride (130 mg) afforded the title compound (14 mg, 18%) as a pale buff solid.

LC/MS [MH+]=446/448, Rt=2.30 min.

### Example 12

### 2-(5-{[5-Chloro-2-(1-methylethyl)-1-benzofuran-7-yl]methyl}-2-furanyl)-5-(1-pyrrolidinylmethyl)-1H-benzimidazole Hydrochloride (E12)

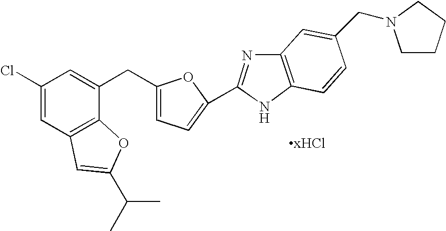

In a manner similar to E10, Step (e), 2-(5-{[5-chloro-2-(1-methylethyl)-1-benzofuran-7-yl]methyl}-2-furanyl)-1H-benzimidazole-5-carbaldehyde (64 mg), pyrrolidine (26 μl) and sodium triacetoxyborohydride (65 mg) afforded the title compound (46 mg, 55%) as a pale buff solid.

LC/MS [MH+]=474/476, Rt=2.43 min.

### Example 13

### 2-(5-{[5-Chloro-2-(1-methylethyl)-1-benzofuran-7-yl]methyl}-2-furanyl)-5-[(4-methyl-1-piperazinyl)methyl]-1H-benzimidazole Hydrochloride (E13)

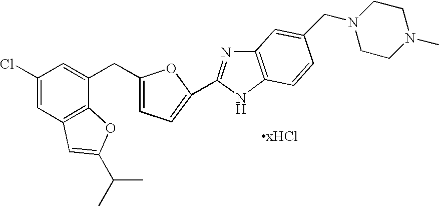

In a manner similar to E10, Step (e) 2-(5-{[5-chloro-2-(1-methylethyl)-1-benzofuran-7-yl]methyl}-2-furanyl)-1H-benzimidazole-5-carbaldehyde (64 mg; may be prepared as described in E10, Step (a)), 1-methylpiperazine (70 μl) and sodium triacetoxyborohydride (130 mg) afforded the title compound (44 mg, 47%) as an off-white solid.

LC/MS [MH+]=503/505, Rt=2.23 min.

### Example 14

### 1-{[5-Chloro-2-(1-methylethyl)-1-benzofuran-7-yl]methyl}-5-methyl-N-{4-[(methylamino)methyl]phenyl}-1H-pyrazole-3-carboxamide (E14)

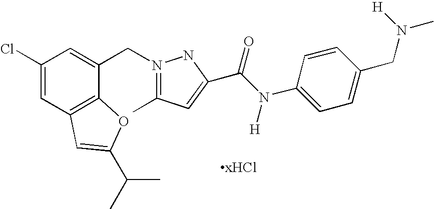

A solution of 1-{[5-chloro-2-(1-methylethyl)-1-benzofuran-7-yl]methyl}-N-(4-formylphenyl)-5-methyl-1H-pyrazole-3-carboxamide (0.06 g, 0.138 mmol) in dry DCM (1.4 ml) was stirred at room temperature under an atmosphere of argon. Methylamine (6.5 μl, 0.165 mmol) was added to the stirred solution. The reaction solution was stirred at room temperature for 30 minutes. Sodium triacetoxyborohydride (0.035 g, 0.165 mmol), acetic acid (0.1 ml) and dry DCM (0.1 ml) were added to the stirred solution. The reaction solution was left stirring at room temperature under an atmosphere of argon overnight. The reaction was monitored by LC-MS. After this time, further methylamine (4 equiv, 21.7 μl, 0.550 mmol), acetic acid (0.1 ml) and sodium triacetoxyborohydride (1 equiv, 0.029 g, 0.138 mmol) were added to the stirred solution. At this stage, in some cases dry THF (2 ml) was also added to the solution to aid solubility. The reaction solution was left stirring at room temperature for a further 2 hours. After this time, the reaction solution was diluted with water (3 ml) and basified to pH 11 using 2M NaOH (0.2 ml). Organics were extracted with DCM (×2). The combined organics were dried over magnesium sulfate, filtered and concentrated under reduced pressure. The resulting residue was purified using MDAP. The purified residue was stirred in HCl in diethyl ether (5 ml) for 15 minutes. After this time the mixture was concentrated under reduced pressure to give 1-{[5-chloro-2-(1-methylethyl)-1-benzofuran-7-yl]methyl}-5-methyl-N-{4-[(methylamino)methyl]phenyl}-1H-pyrazole-3-carboxamide hydrochloride (3 mg, 5%)

Rt=2.39, [M-NMe]: 420, 422

### Examples 15-17

### E15-E17

The following compounds were prepared in an analogous manner to that described in Example 14 (E14):

| Example No. | Structure | Name | LC/MSData |
|---|---|---|---|
| 15 | 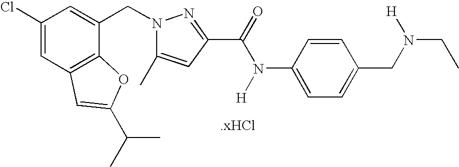 | 1-{[5-Chloro-2-(1-methylethyl)-1-benzofuran-7-yl]methyl}-N-{4-[(ethylamino)methyl]phenyl}-5-methyl-1H-pyrazole-3-carboxamide hydrochloride | Rt =2.42min,[MH⁺]:465 |
| 16 | 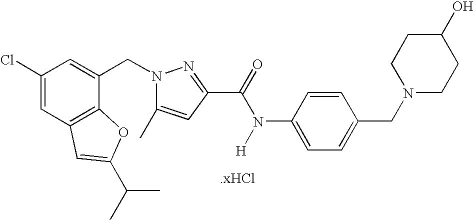 | 1-{[5-Chloro-2-(1-methylethyl)-1-benzofuran-7-yl]methyl}-N-{4-[(4-hydroxy-1-piperidinyl)methyl]phenyl}-5-methyl-1H-pyrazole-3-carboxamide hydrochloride | Rt =2.38min,[MH⁺]:521,523 |
| 17 | 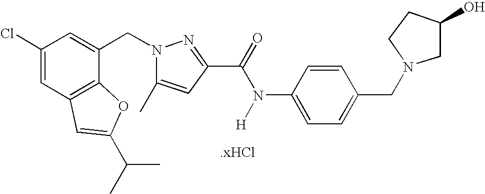 | 1-{[5-Chloro-2-(1-methylethyl)-1-benzofuran-7-yl]methyl}-N-(4-{[(3R)-3-hydroxy-1-pyrrolidinyl]methyl}phenyl)-5-methyl-1H-pyrazole-3-carboxamide hydrochloride | Rt =2.48min,[MH⁺]:507,509 |

### Example 18

### 1-{[5-Bromo-2-(1-methylethyl)-1-benzofuran-7-yl]methyl}-5-methyl-N-4-morpholinyl-1H-pyrazole-3-carboxamide (E18)

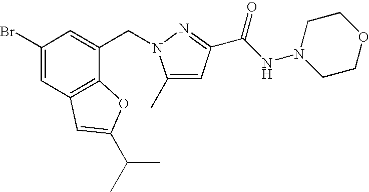

### (a) 1-{[5-Bromo-2-(1-methylethyl)-1-benzofuran-7-yl]methyl}-5-methyl-1H-pyrazole-3-carbonyl Chloride

A solution of 1-{[5-bromo-2-(1-methylethyl)-1-benzofuran-7-yl]methyl}-5-methyl-1H-pyrazole-3-carboxylic acid (189 mg, 0.50 mmol) and thionyl chloride (182 μl, 2.50 mmol) in dry dichloromethane (2 ml) was stirred under reflux for 4 hours. It was allowed to cool and the solvent removed under reduced pressure (u.r.p.). Traces of thionyl chloride were removed by azeotroping with toluene (2 times). This gave acid chloride title compound as a pale buff oil.

### (b) 1-{[5-Bromo-2-(1-methylethyl)-1-benzofuran-7-yl]methyl}-5-methyl-N-4-morpholinyl-1H-pyrazole-3-carboxamide

The acid chloride, 1-{[5-bromo-2-(1-methylethyl)-1-benzofuran-7-yl]methyl}-5-methyl-1H-pyrazole-3-carbonyl chloride was dissolved in dry dichloromethane (2 ml) and the solution cooled in an ice-salt bath. Triethylamine (84 μl, 0.60 mmol) was added followed by a solution of 4-aminomorpholine (58 μl, 0.60 mmol) in dry dichloromethane (0.5 ml). The solution was stirred at 0° C. for 15 minutes then at ambient temperature for 30 minutes. Solvent was removed under reduced pressure (u.r.p.) and ethyl acetate (15 ml) added. The solution was washed with water (5 ml) and saturated sodium bicarbonate solution (5 ml), dried over MgSO₄ and evaporated down under reduced pressure (u.r.p.). The residue was purified by chromatography on silica gel (Biotage SP4®) using 12-100% EtOAc in hexane. Further chromatography (using standard Biotage® apparatus) and 9:1 DCM:methanol took off the product (title compound) (174.0 mg, 75.3%) as a pale cream solid.

LC/MS [M+1]=461, 463.

### Example 19

### 1-{[5-Bromo-2-(1-methylethyl)-1-benzofuran-7-yl]methyl}-5-methyl-1H-pyrazole-3-carboxylic Acid (E19)

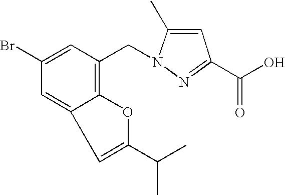

Ethyl 1-{[5-bromo-2-(1-methylethyl)-1-benzofuran-7-yl]methyl}-5-methyl-1H-pyrazole-3-carboxylate (2.52 g, 6.207 mmol), 2N sodium hydroxide solution (12.5 ml, 25 mmol) and ethanol (50 ml) were stirred at room temperature for 1.5 hours. Solvent was removed under reduced pressure (u.r.p.) The solution was acidified to pH 1 with 5N hydrochloric acid and the precipitated white solid filtered off, washed with water and dried in vacuo at 50° C. to yield the title compound (2.21 g, 94.0%) as a white solid.

LC/MS [M+1]=377/379.

### Example 20

### 5-Methyl-1-{[2-(1-methylethyl)-1-benzofuran-7-yl]methyl}-1H-pyrazole-3-carboxylic Acid (E20)

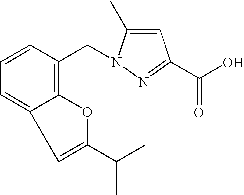

1-{[5-Bromo-2-(1-methylethyl)-1-benzofuran-7-yl]methyl}-5-methyl-1H-pyrazole-3-carboxylate (100 mg, 0.265 mmol) was stirred in dry toluene (0.5 ml). A suspension of Reike zinc in THF (0.5 ml) was added. Reaction solution stirred at room temperature under argon for 20 minutes. Sample removed for LC/MS analysis. (starting material 83%, product 17%). Heated at 40° C. under argon for 17 hours (overnight). Sample removed for LC/MS analysis—no change. Heated to 80° C. under argon and further Reike zinc (0.5 ml) added. Stirred for 2 hours then a sample was removed for LC/MS analysis—27% complete. Further Reike zinc (0.5 ml) added. Reaction stirred at 80° C. under argon for 2 hours. Sample removed for LC/MS analysis—50% complete. Reaction allowed to cool to room temperature then cooled to 0° C. using ice and quenched by the slow addition of water (10 ml). Diluted with EtOAc. Washed with brine (2×10 ml). EtOAc dried over MgSO₄, filtered to remove MgSO₄ and zinc and concentrated under reduced pressure to give a grey oil. Zinc appeared to be still present. Consequently the residue was diluted with EtOAc and filtered through celite. Organics concentrated under reduced pressure to give a grey oil. Dissolved in 50:50 methanol:DMSO and purified by MDAP to give the title compound as a white solid.

LC/MS Rt=2.99 min, [M+1]=299.

### Example 21

### 1-[(5-chloro-2-propyl-1-benzofuran-7-yl)methyl]-5-methyl-1H-pyrazole-3-carboxylic Acid (E21)

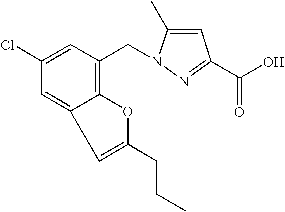

Ethyl 1-[(5-chloro-2-propyl-1-benzofuran-7-yl)methyl]-5-methyl-1H-pyrazole-3-carboxylate (1.32 g, 3.658 mmol), 2N sodium hydroxide solution (7.5 ml, 15.0 mmol) and ethanol (30 ml) were stirred at room temperature for 2 hours. Solvent was removed under reduced pressure (u.r.p.) The solution was acidified to pH 1 with 5N hydrochloric acid and the precipitated white solid filtered off, washed with water and dried in vacuo at 50° C. to yield the title compound (1.196 g, 98.2%) as a white solid.

LC/MS [M+1]=333, 335; [M−1] 331, 333.

### Example 22

### 5-methyl-1-{[2-(1-methylethyl)-5-(methylsulfonyl)-1-benzofuran-7-yl]methyl}-1H-pyrazole-3-carboxylic Acid (E22)

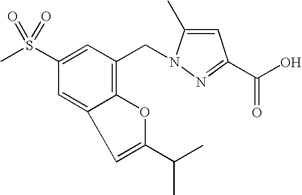

Prepared in a similar manner to 1-[(5-chloro-2-propyl-1-benzofuran-7-yl)methyl]-5-methyl-1H-pyrazole-3-carboxylic acid.

LC/MS Rt 2.54 min, [MH⁺]=377.1.

### Example 23

Sodium 1-[(5-chloro-2-cyclopropyl-1-benzofuran-7-yl)methyl]-5-methyl-1H-pyrazole-3-carboxylate (E23)

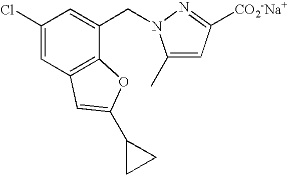

Solution of ethyl 1-[(5-chloro-2-cyclopropyl-1-benzofuran-7-yl)methyl]-5-methyl-1H-pyrazole-3-carboxylate (0.473 g, 1.32 mmol) in EtOH (5.3 ml) was stirred at room temperature under an atmosphere of argon. 2M NaOH aq. soln. (1.98 ml, 3.96 mmol) was added to the solution and the solution heated to 60° C. for 1 hour. Solution was allowed to cool to room temperature and the EtOH was removed under reduced pressure to give a pale yellow coloured solid. Residue was recrystallised using iso-propanol (iPrOH) and a few drops of hexane to give a white coloured solid (0.278 g)

LC/MS Rt=2.98 mins [MH⁺] 331, 333

### Example 1

### 5-{[5-Chloro-2-(1-methylethyl)-1-benzofuran-7-yl]methyl}-2-furancarboxylic Acid (E1)

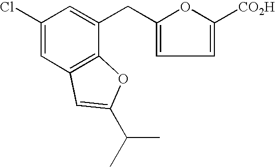

10 mL of 2M NaOH were added to a solution of ethyl 5-{[5-chloro-2-(1-methylethyl)-1-benzofuran-7-yl]methyl}-2-furancarboxylate (3.95 g) in ethanol (100 ml) and left to stand at room temperature for 2 hours. Solvent evaporated (vacced off), dissolved in water (100 ml) and washed with ether (50 ml). Aqueous layer separated, acidified with 2M HCl and extracted with ether (150 ml then 50 ml). Combined extracts dried (MgSO₄), evaporated and triturated with ether and filtered off to give the title compound (2.41 g) as an off-white solid.

LC/MS Rt 3.43 min, [M+H⁺] 319.2

The following compound was prepared in a similar manner.

| Example | Structure | Name | LC/MS data |
|---|---|---|---|
| Example 24(E24) | 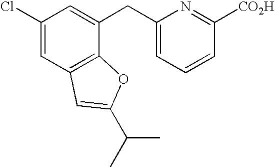 | 6-{[5-Chloro-1-(1-methylethyl)-1-benzofuran-7-yl]methyl}-2-pyridinecarboxylicacid | Rt = 3.23 min,[M + H]⁺ 330.1 |

### Example 25

### 1-{[5-Cyano-2-(1-methylethyl)-1-benzofuran-7-yl]methyl}-5-methyl-1H-pyrazole-3-carboxylic Acid (E25)

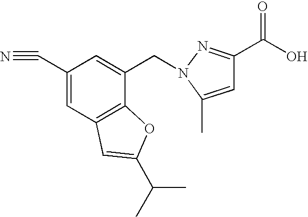

Ethyl 1-{[5-cyano-2-(1-methylethyl)-1-benzofuran-7-yl]methyl}-5-methyl-1H-pyrazole-3-carboxylate (1 g, 2.84 mmol) was dissolved in ethanol (15 ml), NaOH 2M (1.42 ml, 2.84 mmol) was added and the solution was stirred at room temperature for 5 hours. The solvent was evaporated and the residue was diluted with water (100 ml), extracted with diethyl ether (2×60 ml), acidified with 2M HCl and extracted with ethyl acetate (3×100 ml). The combined extracts were dried (MgSO₄), evaporated and azeotroped with toluene to give the title compound as white solid (700 mg).

LC/MS Rt=2.76 min, [MH⁺] 324.2, 325.2, [MH⁻] 322.1, 323.1

### Example 26

### 1-[(5-Chloro-2-cyclohexyl-1-benzofuran-7-yl)methyl]-5-methyl-1H-pyrazole-3-carboxylic Acid (E26)

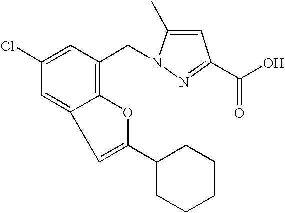

Ethyl 1-[(5-chloro-2-cyclohexyl-1-benzofuran-7-yl)methyl]-5-methyl-1H-pyrazole-3-carboxylate (366 mg, 0.914 mmol) dissolved in EtOH (2 ml). 2M sodium hydroxide solution (0.91 ml, 1.828 mmol) added and reaction stirred at 90° C. for 1 hour. Sample removed for LC/MS analysis—reaction complete. Reaction solution allowed to cool to room temp. Diluted with EtOAc and acidified to pH 1 using 1M hydrochloric acid. Aqueous layer washed further with EtOAc. Organics combined, dried over MgSO₄ and filtered and concentrated to give the title compound (389 mg) as a white solid.

LC/MS Rt 3.40 min, [M+H]=373, 375.

### Example 27

### 1-{[5-Chloro-2-(1,1-dimethylethyl)-1-benzofuran-7-yl]methyl}-5-methyl-1H-pyrazole-3-carboxylic Acid (E27)

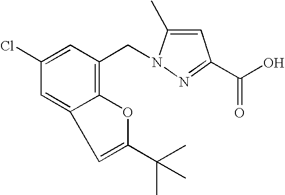

Prepared in a similar manner to 1-[(5-chloro-2-cyclohexyl-1-benzofuran-7-yl)methyl]-5-methyl-1H-pyrazole-3-carboxylic acid (example 26).

LC/MS Rt 3.12 min, [M+H]=347, 349.

### Example 28

### 1-[(5-Chloro-2-cyclohexyl-1-benzofuran-7-yl)methyl]-5-methyl-N-4-morpholinyl-1H-pyrazole-3-carboxamide (E28)

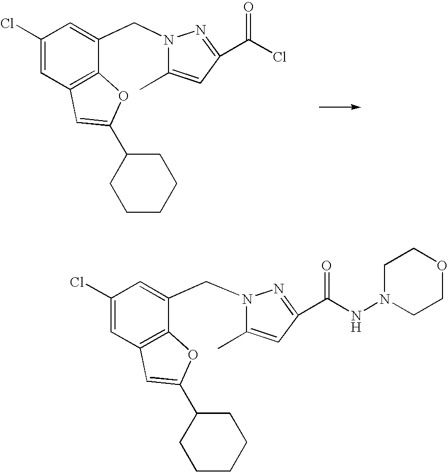

1-[(5-Chloro-2-cyclohexyl-1-benzofuran-7-yl)methyl]-5-methyl-1H-pyrazole-3-carbonyl chloride (158 mg, 0.404 mmol) dissolved in dry DCM (0.8 ml). 4-Aminomorpholine (47 uL) and Et₃N (135 ul, 0.970 mmol) added. Further 1.2 equivalents of 4-amino-morpholine and 2.4 equivalents of Et₃N added. Reaction solution stirred at room temperature under an atmosphere of argon for 17 hours (overnight). Sample removed for LC/MS analysis—no starting material present. Reaction solution diluted with EtOAc and washed with a saturated solution of NaHCO₃ to pH 11. Aqueous layer washed with further EtOAc. Combined organics were dried over MgSO₄filtered and concentrated to give a yellow oil.

The residue was chromatographed [SiO₂, Hex:EtOAc 40-70%] to give the title compound. (94 mg, 51%).

LC/MS Rt=3.39 mins [M+H] 457, 459.

The following example was prepared using similar conditions.

| Example | Structure | Name | LC/MS data |
|---|---|---|---|
| E29 | 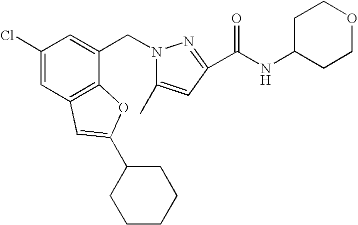 | 1-[(5-Chloro-2-cyclohexyl-1-benzofuran-7-yl)methyl]-5-methyl-N-(tetrahydro-2H-pyran-4-yl)-1H-pyrazole-3-carboxamide | Rt 3.62 min, [M + H]456, 458 |

### Example 30

### 1-[(5-chloro-2-propyl-1-benzofuran-7-yl)methyl]-5-methyl-N-4-morpholinyl-1H-pyrazole-3-carboxamide (E30)

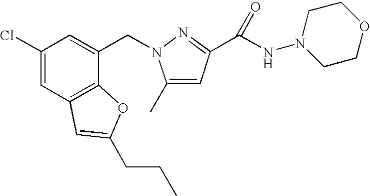

### (a) 1-[(5-chloro-2-propyl-1-benzofuran-7-yl)methyl]-5-methyl-1H-pyrazole-3-carbonyl Chloride

A solution of 1-[(5-chloro-2-propyl-1-benzofuran-7-yl)methyl]-5-methyl-1H-pyrazole-3-carboxylic acid (133 mg, 0.40 mmo) and thionyl chloride (145 μl, 2.0 mmol) in dichloromethane (2.5 ml) was stirred under reflux for 4 hours. It was allowed to cool and solvent removed under reduced pressure (u.r.p.). Traces of thionyl chloride were removed by azeotroping with toluene (2×). This gave the acid chloride as a white solid.

### (b) 1-[(5-chloro-2-propyl-1-benzofuran-7-yl)methyl]-5-methyl-N-4-morpholinyl-1H-pyrazole-3-carboxamide

The acid chloride (1-[(5-chloro-2-propyl-1-benzofuran-7-yl)methyl]-5-methyl-1H-pyrazole-3-carbonyl chloride) was dissolved in dry CH₂Cl₂ (2 ml) and the solution cooled in an ice-salt bath. Triethylamine (67 μl, 0.48 mmol) was added followed by a solution of 4-aminomorpholine (46 μl, 0.48 mmol) in dichloromethane (0.5 ml). The solution was stirred at 0° C. for 15 minutes then at ambient temperature for 1 hour. Solvent was removed under reduced pressure (u.r.p.) and EtOAc (10 ml) added. The solution was washed with water (3 ml) and saturated sodium bicarbonate solution (5 ml) and saturated NaHCO₃ (5 ml), dried over MgSO₄ and evaporated down under reduced pressure (u.r.p.). The residue was purified by MDAP (mass-directed autopreparation) to give the title compound (92.5 mg, 55.5%) as a white solid.

LC/MS [M+1]=417, 419.

### Example 31 & 32

### 1-[(5-Chloro-2-cyclopropyl-1-benzofuran-7-yl)methyl]-5-methyl-N-4-morpholinyl-1H-pyrazole-3-carboxamide (E31) and 4-({1-[(5-chloro-2-cyclopropyl-1-benzofuran-7-yl)methyl]-5-methyl-1H-pyrazol-3-yl}carbonyl)morpholine (E32)

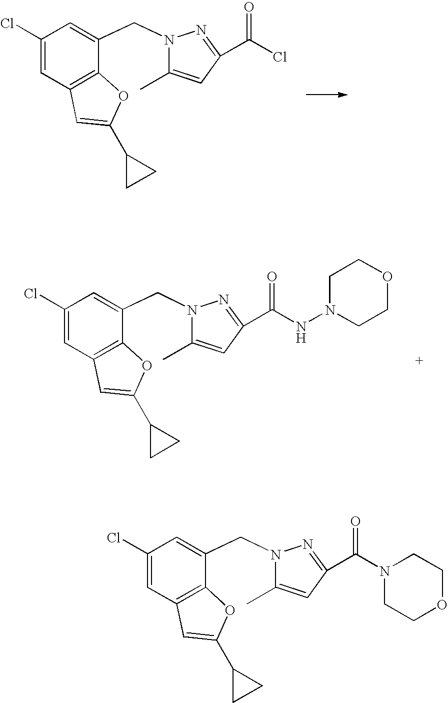

A solution of 1-[(5-chloro-2-cyclopropyl-1-benzofuran-7-yl)methyl]-5-methyl-1H-pyrazole-3-carbonyl chloride (0.41 mmol) in dry DCM (2.0 ml) was stirred at room temperature under an atmosphere of argon. Et₃N (0.114 ml, 0.82 mmol) and 4-amino-morpholine (0.06 ml, 0.62 mmol) were added and the mixture stirred at room temperature overnight. Mixture was diluted with DCM and washed with water (×2). Organics were dried over MgSO₄ Filtered and concentrated under reduced pressure to give a yellow oily coloured solid. The residue was chromatographed [SiO₂, Hex:EtOAc 50-100%] to give 1-[(5-chloro-2-cyclopropyl-1-benzofuran-7-yl)methyl]-5-methyl-N-4-morpholinyl-1H-pyrazole-3-carboxamide, example 31. (0.082 g)

LC/MS Rt=2.92 mins [MH⁺] 415,417

As second compound, identified as 4-({1-[(5-chloro-2-cyclopropyl-1-benzofuran-7-yl)methyl]-5-methyl-1H-pyrazol-3-yl}carbonyl)morpholine, example 32, was also obtained from the reaction. (0.013 g). LC/MS Rt 3.11 mins, [MH⁺] 400, 402

### Example 33

### 1-{[5-chloro-2-(1-methylethyl)-1-benzofuran-7-yl]methyl}-5-methyl-N-4-piperidinyl-1H-pyrazole-3-carboxamide Hydrochloride (E33)

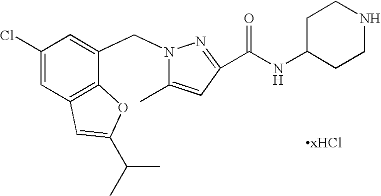

Solution of 1,1-dimethylethyl 4-{[(1-{[5-chloro-2-(1-methylethyl)-1-benzofuran-7-yl]methyl}-5-methyl-1H-pyrazol-3-yl)carbonyl]amino}-1-piperidinecarboxylate (0.286 g, 0.56 mmol) in HCl in Et₂O (4.0 ml, 1M) was stirred at room temperature for 4 hours. LC/MS showed removal of the tert-butoxy (BOC) group. Solvent was removed under reduced pressure and residue triturated using Et₂O to give a white solid (0.095 g).

LC/MS Rt=2.24 mins [MH⁺] 415, 417.

The following compounds was prepared in a similar manner from the appropriate amines.

| Example | Structure | Name | Data |
|---|---|---|---|
| E34 | 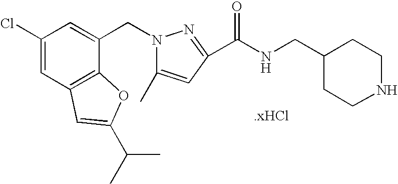 | 1-{[5-Chloro-2-(1-methylethyl)-1-benzofuran-7-yl]methyl}-5-methyl-N-(4-piperidinylmethyl)-1H-pyrazole-3-carboxamidehydrochloride | Rt = 2.28mins,[MH⁺] 429,431 |

### Example 35

### 1-{[5-chloro-2-(1-methylethyl)-1-benzofuran-7-yl]methyl}-N-(1-ethyl-4-piperidinyl)-5-methyl-1H-pyrazole-3-carboxamide Hydrochloride (E35)

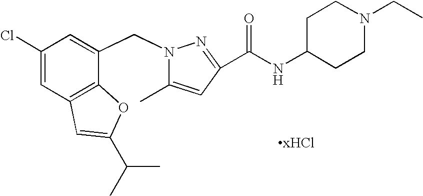

Solution of 1-{[5-chloro-2-(1-methylethyl)-1-benzofuran-7-yl]methyl}-5-methyl-N-4-piperidinyl-1H-pyrazole-3-carboxamide hydrochloride in EtOAc was treated with solid K₂CO₃ to neutralise HCl. Inorganics were filtered off and solvent removed under reduced pressure to give an oily solid (0.134 g).

The residue (0.134 g) was dissolved in dry DCM (1.3 ml) and the solution stirred at room temperature under an atmosphere of argon. Acetaldehyde (0.035 ml, 0.64 mmol) was added to the solution. 4 Å molecular sieve (MS) (0.050 g) were added to the solution. The mixture was stirred for 30 minutes. After this time, NaBH(OAc)₃ (0.101 g, 048 mmol) was added and the mixture stirred at room temperature for 18 hours (overnight). After this time LC/MS showed predominantly product. Water was added to the mixture and organics extracted into DCM (×2). The combined organics were dried over MgSO₄, filtered and concentrated under reduced pressure to give a brown coloured oil. The residue was chromatographed [SiO₂, DCM/MeOH, 0-5%). The residue was treated with HCl in Et₂O (1M) to give the title compound (0.032 g).

LC/MS Rt=2.33 mins, [MH⁺] 443, 445

The following compound was prepared in a similar manner, except that the residue was purified using MDAP.

| | | | LC/MS |
|---|---|---|---|
| Example | Structure | Name | Data |
| E36 | 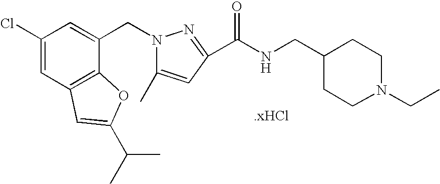 | 1-{[5-chloro-2-(1-methylethyl)-1-beenzofuran-7-yl]methyl}-N-[(1-ethyl-4-piperidinyl)methyl]-5-methyl-1H-pyrazole-3-carboxamidehydrochloride | Rt = 2.39mins,[MH⁺] 457,459 |

### Example 37

### 5-{[5-Chloro-2-(1-methylethyl)-1-benzofuran-7-yl]methyl}-2-furancarboxamide (E37)

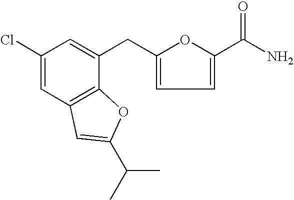

Oxalyl chloride (0.2 ml) was added to a solution of 5-{[5-chloro-2-(1-methylethyl)-1-benzofuran-7-yl]methyl}-2-furancarboxylic acid (100 mg) and one drop of DMF in DCM (5 ml) and left at room temperature for 30 minutes. Evaporated (vacced off), azeotroped with toluene (5 ml) then dissolved in ether (5 ml) and conc. aqueous ammonia (2 ml) added with stirring. Stirred for 30 minutes and the solid filtered off and washed with water and ether to give the title compound (62 mg).

LC/MS Rt 3.21 min, [M+H]⁺ 318.2, 320.2

The following compounds were prepared in a similar manner by reaction of the appropriate acid with oxalyl chloride then with isopropylamine or 0.88 aqueous ammonia or N,N-dimethylhydrazine.

| | | | LC/MS |
|---|---|---|---|
| Example | Structure | Name | data |
| E38 | 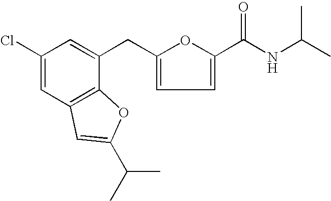 | 5-{[5-Chloro-2-(1-methylethyl)-1-benzofuran-7-yl]methyl}-N-(1-methylethyl)-2-furancarboxamide | Rt = 3.65min,[M + H]⁺360.2,362.2 |
| E39 | 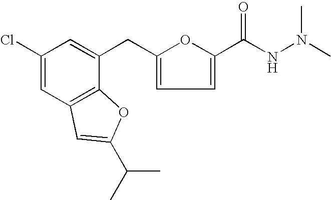 | 5-{[5-Chloro-2-(1-methylethyl)-1-benzofuran-7-yl]methyl}-N′,N′-dimethyl-2-furancarbohydrazide | Rt = 3.21min,[M + H]⁺361.2,363.2 |
| E40 | 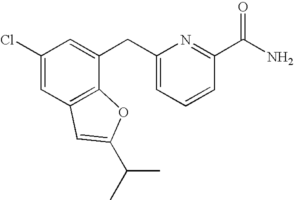 | 6-{[5-Chloro-2-(1-methylethyl)-1-benzofuran-7-yl]methyl}-2-pyridinecarboxamide | Rt = 3.46min,[M + H]⁺329.1 |
| E41 | 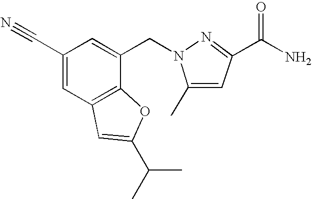 | 1-{[5-Cyano-2-(1-methylethyl)-1-benzofuran-7-yl]methyl}-5-methyl-1H-pyrazole-3-carboxamide | Rt = 2.72min[M + H]⁺323.3 |

### Example 42

5-{[5-Chloro-2-(1-methylethyl)-1-benzofuran-7-yl]methyl}-N-4-morpholinyl-2-furancarboxamide (E42)

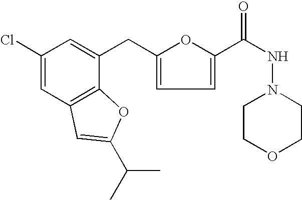

Oxalyl chloride (0.2 ml) was added to a solution of 5-{[5-chloro-2-(1-methylethyl)-1-benzofuran-7-yl]methyl}-2-furancarboxylic acid (106 mg) and one drop of DMF in DCM (5 ml) and left at room temperature for 30 minutes. Evaporated (vacced off) then dissolved in DCM (5 ml) and a solution of 4-aminomorpholine (51 mg, 0.5 mmol) in pyridine (0.5 ml) added. Stirred for 30 minutes then diluted with ether (30 ml) and washed with water (20 ml). Organic phase dried (MgSO₄), evaporated and chromatographed on silica gel (Biotage®) with 50-100% EtOAc in hexane. Triturated with ether and filtered off to give the title compound (52 mg) as a white solid.

LC/MS Rt 3.19 min, [M+H]⁺ 403.2, 405.2

The following compounds were prepared in a similar manner by reaction of the appropriate acid with oxalyl chloride then treatment with an amine and pyridine.

| GSK No. | Structure | Name | LC/MS data |
|---|---|---|---|
| E43 | 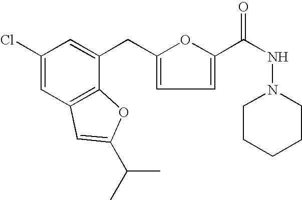 | 5-{[5-Chloro-2-(1-methylethyl)-1-benzofuran-7-yl]methyl}-N-1-piperidinyl-2-furancarboxamide | Rt = 3.55min,[M + H]⁺401.2,403.2 |
| E44 | 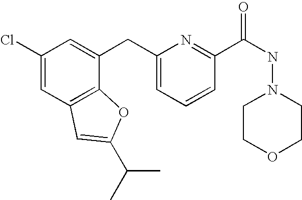 | 6-{[5-Chloro-2-(1-methylethyl)-1-benzofuran-7-yl]methyl}-N-4-morpholinyl-2-pyridinecarboxammide | Rt = 3.41min,[M + H]⁺414.1 |
| D69 | 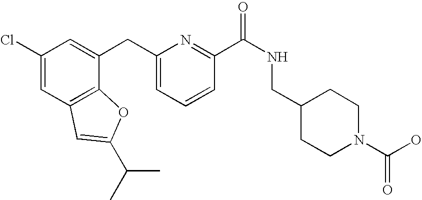 | 1,1-Dimethylethyl 4-({[(6-{[5-chloro-2-(1-methylethyl)-1-benzofuran-7-yl]methyl}-2-pyridinyl)carbonyl]amino}methyl)-1-piperidinecarboxylate | Rt = 3.80min,[M + H −BOC]⁺474.1 |
| E45 | 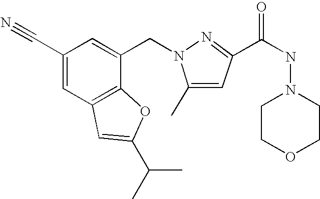 | 1-{[5-Cyano-2-(1-methylethyl)-1-benzofuran-7-yl]methyl}-5-methyl-N-4-morpholinyl-1H-pyrazole-3-carboxamide | Rt = 2.74min[M + H]⁺408.2 |
| D70 | 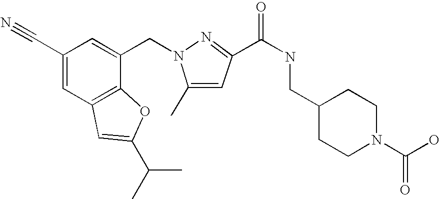 | 1,1-Dimethylethyl 4({[(1-{[5-cyano-2-(1-methylethyl)-1-benzofuran-7-yl]methyl}-5-methyl-1H-pyrazol-3-yl)carbonyl]amino}methyl)-1-piperidinecarboxylate | Rt = 3.45min[M + H −BOC]⁺420.3 |

### Example 46

### 1-{[5-Cyano-2-(1-methylethyl)-1-benzofuran-7-yl]methyl}-5-methyl-N-(4-piperidinylmethyl)-1H-pyrazole-3-carboxamide Hydrochloride (E46)

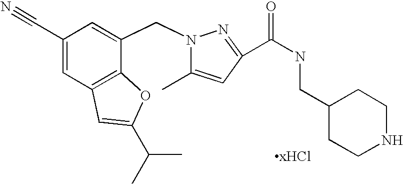

1,1-dimethylethyl 4-({[(1-{[5-cyano-2-(1-methylethyl)-1-benzofuran-7-yl]methyl}-5-methyl-1H-pyrazol-3-yl)carbonyl]amino}methyl)-1-piperidinecarboxylate (90 mg) and 4M HCl in dioxane (4 ml) were mixed together and stirred at room temperature for 2 hours. The mixture was evaporated, diluted with toluene (10 ml) and re-evaporated then triturated with ether to give the title compound (70 mg) as a white solid.

LC/MS Rt 2.13 min [M+H]⁺ 420.3

The following compound was prepared using a similar procedure.

| | | | |
|---|---|---|---|
| E47 | 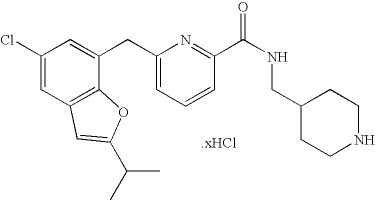 | 6-{[5-Chloro-2-(1-methylethyl)-1-benzofuran-7-yl]methyl}-N-(4-piperidinylmethyl)-2-pyridinecarboxamidehydrochloride | Rt = 2.51min[M + H]⁺426.2 |
| E48 | 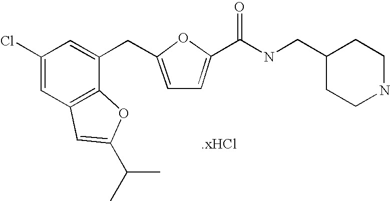 | 5-{[5-Chloro-2-(1-methylethyl)-1-benzofuran-7-yl]methyl}-N-(4-pyridinylmethyl)-2-furancarboxamidehydrochloride | Rt = 2.32min, [MH⁺]415.2,417.2 |

### Example 49

### 1-{[5-Bromo-2-(1-methylethyl)-1-benzofuran-7-yl]methyl}-N-{4-[(4-hydroxy-1-piperidinyl)methyl]phenyl}-5-methyl-1H-pyrazole-3-carboxamide Hydrochloride (E49)

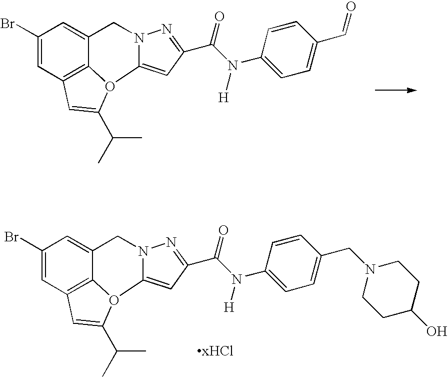

To a stirring solution of 1-{[5-bromo-2-(1-methylethyl)-1-benzofuran-7-yl]methyl}-N-(4-formylphenyl)-5-methyl-1H-pyrazole-3-carboxamide (96 mg, 0.2 mmol) in dry THF (4 ml) was added 4-piperidinol (84 mg, 0.83 mmol) and the solution stirred under argon for 1 hour. Sodium triacetoxyborohydride (170 g, 0.8 mmol) was added and the mixture stirred at 50° C. for 2 hours. EtOAc (15 ml) and brine (15 ml) were added and the aqueous layer separated and extracted with EtOAc (5 ml). The combined organic layers were dried over MgSO₄ and evaporated down under reduced pressure (u.r.p.). The residue was purified by MDAP and dissolved in DCM (˜3 ml). 1M HCl in ether (˜3 ml) was added and the solvent removed under reduced pressure (u.r.p.). the residue was washed with ether and dried in vacuo to give the title compound (35.3 mg, 29.3%) as a pale cream solid.

LC/MS [M+1] 565, 567.

### Examples 50-52

### E50-E52

The following compounds were prepared in an analogous manner to that described in Example 49 (E49). After MDAP all samples were impure. All recrystallised from isopropanol to give the title compounds.

| Example No. | Structure | Name | LC/MSData |
|---|---|---|---|
| E50 | 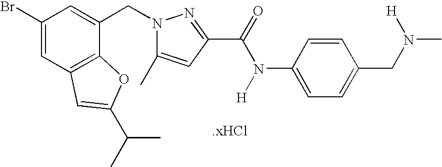 | 1-{[5-Bromo-2-(1-methylethyl)-1-benzofuran-7-yl]methyl}-5-methyl-N-{4-[(methylamino)methyl]phenyl}-1H-pyrazole-3-carboxamidehydrochloride | [M + 1] 464,466 (loss ofCH₃NH₂) |
| E51 | 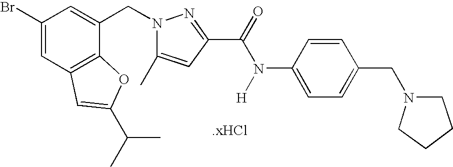 | 1-{[5-Bromo-2-(1-methylethyl)-1-benzofuran-7-yl]methyl}-5-methyl-N-[4-(1-pyrrolidinylmethyl)phenyl]-1H-pyrazole-3-carboxamidehydrochloride | [M + 1] 535,537 and464, 466 |
| E52 | 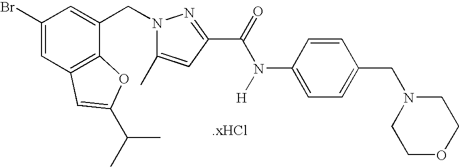 | 1-{[5-Bromo-2-(1-methylethyl)-1-benzofuran-7-yl]methyl}-5-mthyl-N-[4-(4-morpholinylmethyl)phenyl]-1H-pyrazole-3-carboxamidehydrochloride | [M + 1] 551,553 and464, 466 |

### Example 53

### 5-{[5-Chloro-2-(1-methylethyl)-1-benzofuran-7-yl]methyl}-N-[4-(1-pyrrolidinylmethyl)phenyl]-2-furancarboxamide (E53)

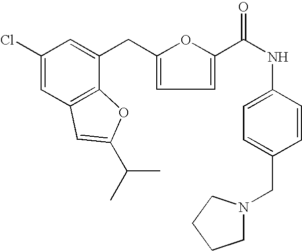

Sodium triacetoxyborohydride (85 mg, 0.4 mmol) was added to a stirred solution of 5-{[5-chloro-2-(1-methylethyl)-1-benzofuran-7-yl]methyl}-N-(4-formylphenyl)-2-furancarboxamide (84 mg, 0.2 mmol) and pyrrolidine (28 mg, 0.4 mmol) in THF (3 ml) and stirred at room temperature for 5 hours. The mixture was diluted with ethyl acetate/water (30 ml of each) and the organic phase dried (magnesium sulphate), evaporated, triturated with ether and the solid filtered off to give the title compound (57 mg).

LC/MS: Rt=2.61 min, [M+H]⁺ 477.2

### Example 54

### 5-{[5-Chloro-2-(1-methylethyl)-1-benzofuran-7-yl]methyl}-N-[4-(4-morpholinylmethyl)phenyl]-2-furancarboxamide Hydrochloride (E54)

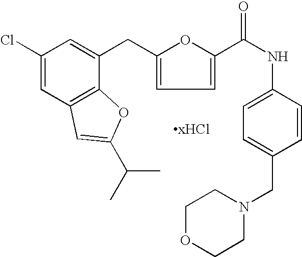

Sodium triacetoxyborohydride (85 mg, 0.4 mmol) was added to a stirred solution of 5-{[5-chloro-2-(1-methylethyl)-1-benzofuran-7-yl]methyl}-N-(4-formylphenyl)-2-furancarboxamide (84 mg, 0.2 mmol) and morpholine (34 mg, 0.4 mmol) in THF (3 ml) and stirred at room temperature for 5 hours. The mixture was diluted with ethyl acetate/water (30 ml of each) and the organic phase dried (magnesium sulphate), evaporated and purified by flash chromatography on a Biotage® column eluting with ethyl acetate. The product was dissolved in dichloromethane (2 ml) and treated with 1M hydrogen chloride in ether (0.5 ml). After evaporation and trituration with ether there was obtained the title compound as a pale yellow solid (29 mg).

LC/MS: Rt=2.57 min, [M+H]⁻ 491.1, 493.1

### Example 55

### 5-{[5-Chloro-2-(1-methylethyl)-1-benzofuran-7-yl]methyl}-N-{4-[(4-hydroxy-1-piperidinyl)methyl]phenyl}-2-furancarboxamide Hydrochloride (E55)

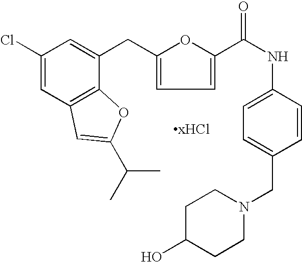

Sodium triacetoxyborohydride (85 mg, 0.4 mmol) was added to a stirred solution of 5-{[5-chloro-2-(1-methylethyl)-1-benzofuran-7-yl]methyl}-N-(4-formylphenyl)-2-furancarboxamide (84 mg, 0.2 mmol) and 4-hydroxypiperidine (40 mg, 0.4 mmol) in THF (3 ml) and stirred at room temperature for 5 hours. The mixture was diluted with ethyl acetate/water (30 ml of each) and the organic phase dried (magnesium sulphate), evaporated and purified by flash chromatography on a Biotage® column eluting with 1:1 ethyl acetate/hexane to remove impurities and changing to 1:4 methanol/dichloromethane to elute the product. The product was dissolved in dichloromethane (2 ml) and treated with 1M hydrogen chloride in ether (0.5 ml). After evaporation and trituration with ether there was obtained the title compound as a pale yellow solid (39 mg).

LC/MS: Rt=2.50 min, [M+H]⁻ 505.1, 507.1

### Example 56

### 5-{[5-Chloro-2-(1-methylethyl)-1-benzofuran-7-yl]methyl}-N-{4-[(ethylamino)methyl]phenyl}-2-furancarboxamide Hydrochloride (E56)

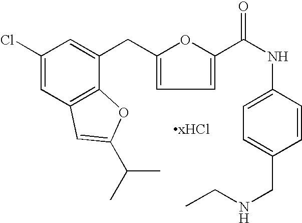

Sodium triacetoxyborohydride (85 mg, 0.4 mmol) was added to a stirred solution of 5-{[5-chloro-2-(1-methylethyl)-1-benzofuran-7-yl]methyl}-N-(4-formylphenyl)-2-furancarboxamide (84 mg, 0.2 mmol) and 2M ethylamine in THF (0.2 ml, 0.4 mmol) in THF (3 ml) and stirred at room temperature for 24 hours. Sodium borohydride (19 mg, 0.5 mmol) was added and after a further 5 hours ethanol (1 ml) was added and the mixture stirred for 16 hours. The mixture was diluted with ethyl acetate/water (30 ml of each) and the organic phase dried (magnesium sulphate), evaporated and purified by flash chromatography on a Biotage column eluting with 1:1 ethyl acetate/hexane to remove impurities and changing to 1:2 methanol/dichloromethane to elute the product. The product was dissolved in dichloromethane (3 ml) and treated with 1M hydrogen chloride in ether (1 ml). After evaporation and trituration with ether there was obtained the title compound as a white solid (60 mg).

LC/MS: Rt=2.54 min, [M+H]⁻ 449.1

### Example 57

### 1,1-Dimethylethyl (5-{[5-chloro-2-(1-methylethyl)-1-benzofuran-7-yl]methyl}-2-furanyl)carbamate (E57)

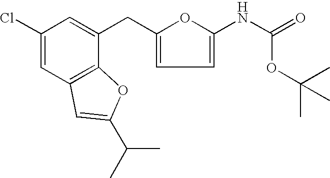

A mixture of 5-{[5-chloro-2-(1-methylethyl)-1-benzofuran-7-yl]methyl}-2-furancarboxylic acid (80 mg, 0.25 mmol), triethylamine (28 mg, 0.28 mmol) and diphenylphosphoryl azide (76 mg, 0.275 mmol) in t-butanol (3 ml) was stirred and heated at 90° C. for 5 hours then evaporated to dryness. The residue was purified by flash chromatography on a Biotage® column eluting with 1:19 ethyl acetate/hexane to give the title compound as a white solid (48 mg).

LC/MS: Rt=4.01 min, [M+H]⁺ 390.2

### Description 71

### 1,1-Dimethylethyl 4-({[(5-{[5-chloro-2-(1-methylethyl)-1-benzofuran-7-yl]methyl}-2-furanyl)carbonyl]amino}methyl)-1-piperidinecarboxylate (D71)

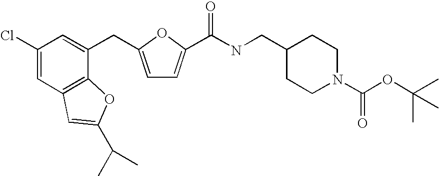

5-{[5-chloro-2-(1-methylethyl)-1-benzofuran-7-yl]methyl}-2-furancarboxylic acid (100 mg, 0.314 mmol), oxalyl chloride (0.2 ml) and DMF (1 drop) in DCM (4 ml) were stirred at room temperature for ½ hour. The solvent was then evaporated and azeotroped with toluene. To the residue were added 1,1-dimethylethyl 4-(aminomethyl)-1-piperidinecarboxylate (80 mg, 0.376 mmol), pyridine(0.1 ml), and DCM (2 ml).The resulting mixture was stirred at room temperature for 1 hour and washed with water (4 ml) using a phase separator cartridge. The organic phase was evaporated and the residue was purified on the Biotage SP4® to give the title compound as clear oil (124 mg).

LC/MS Rt=3.99 min, [MH⁺] 515.2

The following compounds were prepared in a similar manner to 1,1-Dimethylethyl 4-({[(5-{[5-chloro-2-(1-methylethyl)-1-benzofuran-7-yl]methyl}-2-furanyl)carbonyl]amino}methyl)-1-piperidinecarboxylate from the appropriate intermediates.

Hydrochloride salts were prepared by diluting the free base in MeOH (5 ml) and stirring it with 1.0M HCl in diethyl ether (2 ml). The solvent was evaporated and the products were obtained by trituration of the solid with diethyl ether.

| Example | Structure | Name | LC/MS data |
|---|---|---|---|
| E58 | 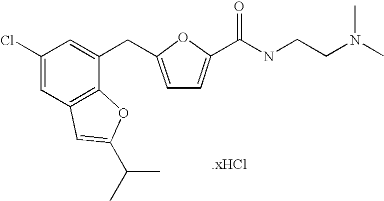 | 5-{[5-Chloro-2-(1-methylethyl)-1-benzofuran-7-yl]methyl}-N-[2-(dimethylamino)ethyl]-2-furancarboxamidehydrochloride | Rt = 2.32 min,[MH⁺] 389.2,391.2 |
| E59 | 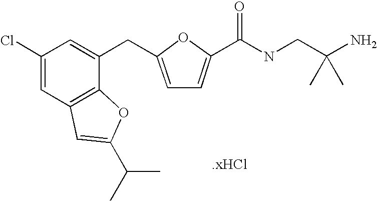 | N-(2-Amino-2-methylpropyl)-5-{[5-chloro-2-(1-methylethyl)-1-benzofuran-7-yl]methyl}-2-furancarboxamidehydrochloride | Rt = 2.36 min,[MH⁺] 389.2,391.2 |

### Example 60

### 1-{[5-chloro-2-(1-methylethyl)-1-benzofuran-7-yl]methyl}-5-methyl-N-(tetrahydro-2H-pyran-4-yl)-1H-pyrazole-3-carboxamide (E60)

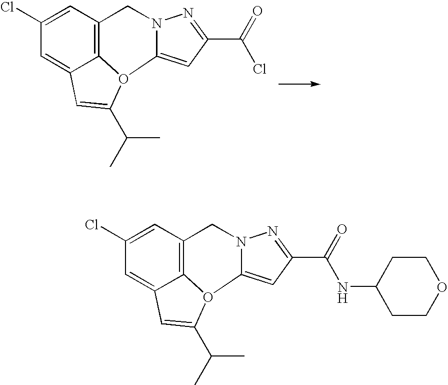

A solution of 1-{[5-chloro-2-(1-methylethyl)-1-benzofuran-7-yl]methyl}-5-methyl-1H-pyrazole-3-carbonyl chloride (0.159 g, 0.452 mmol), tetrahydro-2H-pyran-4-amine (0.055 g, 0.544 mmol) and triethylamine (0.075 ml, 0.542 mmol) in dry DCM (1 ml) was stirred at room temperature under an atmosphere of argon for 2 hours. The reaction was monitored by LC-MS. In some cases, further amine (0.5 equiv) and triethylamine (1 equiv) were added and stirring for a maximum of 17 hours (overnight) was required to progress the reaction further to completion. The reaction mixture was partitioned between EtOAc (50 ml) and NaHCO₃ (10 ml, sat. aq solution). The organics were dried over magnesium sulfate, filtered and concentrated under reduced pressure to give an oil. The resulting residue was triturated using hexane to give a white solid, 1-{[5-chloro-2-(1-methylethyl)-1-benzofuran-7-yl]methyl}-5-methyl-N-(tetrahydro-2H-pyran-4-yl)-1H-pyrazole-3-carboxamide (0.17 g, 92%).

LC/MS Rt=3.19 min, [MH⁺] 416,418

The following compound was prepared using the above procedure:

| Example | Structure | Name | LC/MS Data |
|---|---|---|---|
| E61 | 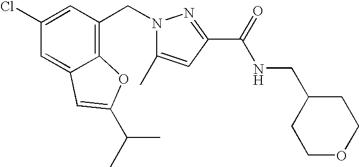 | 1-{[5-Chloro-2-(1-methylethyl)-1-benzofuran-7-yl]methyl}-5-methyl-N-(tetrahydro-2H-pyran-4-ylmethyl)-1H-pyrazole-3-carboxamide | Rt = 3.26 min,[MH⁺] 430,432 |

### Examples 62 & 63

### cis-N-(1-{[5-Chloro-2-(1-methylethyl)-1-benzofuran-7-yl]methyl}-5-methyl-1H-pyrazol-3-yl)-4-hydroxycyclohexanecarboxamide (E62) and trans-N-(1-{[5-chloro-2-(1-methylethyl)-1-benzofuran-7-yl]methyl}-5-methyl-1H-pyrazol-3-yl)-4-hydroxycyclohexanecarboxamide (E63)

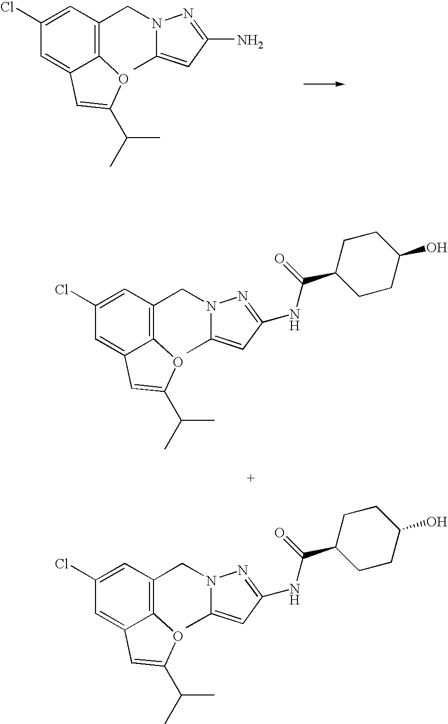

A mixture of 1-{[5-chloro-2-(1-methylethyl)-1-benzofuran-7-yl]methyl}-5-methyl-1H-pyrazol-3-amine (0.150 g, 0.495 mmol), EDAC (0.114 g, 0.594 mmol), HOBt (0.080 g, 0.594 mmol) and cis-4-hydroxycyclohexanecarboxylic acid (0.086 g, 0.594 mmol) in dry DMF (1.7 ml) was stirred at room temperature under an atmosphere of argon for 1 hour. The reaction was monitored by LC-MS. After this time, further cis-4-hydroxycyclohexanecarboxylic acid (0.5 equiv) was added and the reaction solution was stirred for 72 hours (over weekend). The reaction mixture was partitioned between EtOAc and NaHCO₃ (sat. aq solution). The organics were washed with water (3×20 ml), dried over magnesium sulfate, filtered and concentrated under reduced pressure to give a colourless oil. The residue was chromatographed [SiO₂, EtOAc:Hexane 50-75%] to give cis-N-(1-{[5-chloro-2-(1-methylethyl)-1-benzofuran-7-yl]methyl}-5-methyl-1H-pyrazol-3-yl)-4-hydroxycyclohexanecarboxamide (0.048 g) (E62) LC/MS Rt=3.02 min, [MH+] 430,432 and trans-N-(1-{[5-chloro-2-(1-methylethyl)-1-benzofuran-7-yl]methyl}-5-methyl-1H-pyrazol-3-yl)-4-hydroxycyclohexanecarboxamide (0.012 g) (E63) Rt=2.99 min, [MH⁺] 430, 432.

### Example 64

### 1-{[5-Chloro-2-(1-methylethyl)-1-benzofuran-7-yl]methyl}-5-methyl-N-[trans-4-(methyloxy)cyclohexyl]-1H-pyrazole-3-carboxamide (E64)

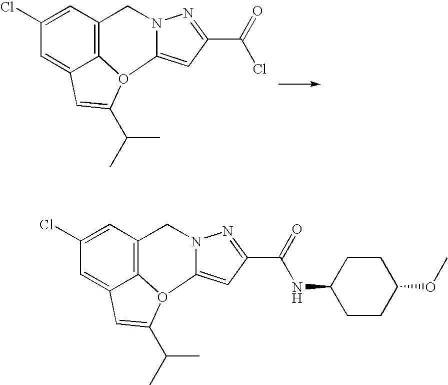

A solution of 1-{[5-chloro-2-(1-methylethyl)-1-benzofuran-7-yl]methyl}-5-methyl-1H-pyrazole-3-carbonyl chloride (0.159 g, 0.452 mmol), trans-4-(methyloxy)cyclohexanamine (0.091 g, 0.542 mmol) and triethylamine (0.126 ml, 1.084 mmol) in dry DCM (1 ml) was stirred at room temperature under an atmosphere of argon for 17 hours (overnight). The reaction was monitored by LC-MS. Further trans-4-(methyloxy)cyclohexanamine (0.038 g, 0.226 mmol) was added and the reaction was stirred for 17 hours (overnight). The reaction mixture was partitioned between EtOAc and NaHCO₃ (sat. aq soln). The organics were dried over magnesium sulfate, filtered and concentrated under reduced pressure to give a white solid. The solid was chromatographed [SiO₂, EtOAc:Hexane 25-50%] to give 1-{[5-chloro-2-(1-methylethyl)-1-benzofuran-7-yl]methyl}-5-methyl-N-[trans-4-(methyloxy)cyclohexyl]-1H-pyrazole-3-carboxamide (0.104 g, 52%).

LC/MS Rt=3.45 min, [MH⁺] 444,446

### Example 65

### 1-{[5-Chloro-2-(1-methylethyl)-1-benzofuran-7-yl]methyl}-N-[trans-4-(ethyloxy)cyclohexyl]-5-methyl-1H-pyrazole-3-carboxamide (E65)

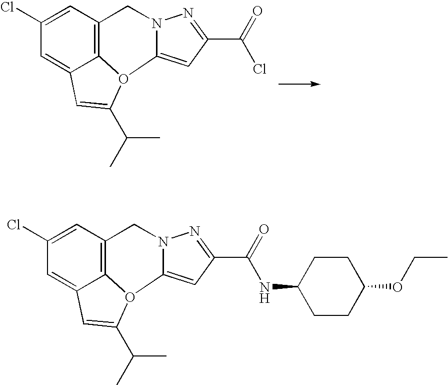

A solution of 1-{[5-chloro-2-(1-methylethyl)-1-benzofuran-7-yl]methyl}-5-methyl-1H-pyrazole-3-carbonyl chloride (0.072 g, 0.205 mmol), trans-4-(ethyloxy)cyclohexanamine hydrochloride (0.037 g, 0.205 mmol) and triethylamine (0.068 ml, 0.492 mmol) in dry DCM (0.4 ml) was stirred at room temperature under an atmosphere of argon for 17 hours (overnight). The reaction was monitored by LC-MS. Further triethylamine (0.028 ml, 0.205 mmol) was added and the reaction was stirred for 17 hours (overnight). The reaction mixture was partitioned between EtOAc and NaHCO₃ (sat. aq solution). The organics were dried over magnesium sulfate, filtered and concentrated under reduced pressure to give a white solid. The solid was chromatographed [SiO₂, EtOAc:Hexane 25-50%] to give 1-{[5-chloro-2-(1-methylethyl)-1-benzofuran-7-yl]methyl}-N-[trans-4-(ethyloxy)cyclohexyl]-5-methyl-1H-pyrazole-3-carboxamide (0.058 g, 62%).

LC/MS Rt=3.59 min, [MH⁺] 458,460

### Example 66

### N-(1-{[5-chloro-2-(1-methylethyl)-1-benzofuran-7-yl]methyl}-5-methyl-1H-pyrazol-3-yl)-4-[(ethylamino)methyl]benzamide (E66)

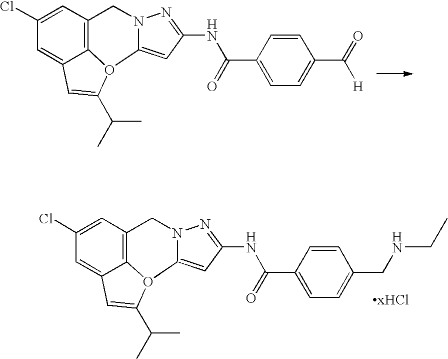

To a solution of N-(1-{[5-chloro-2-(1-methylethyl)-1-benzofuran-7-yl]methyl}-5-methyl-1H-pyrazol-3-yl)-4-formylbenzamide (0.175 g, 0.402 mmol) in dry DCM (4 ml), ethylamine (0.241 ml, 0.482 mmol) and acetic acid (0.1 ml) were added. The reaction solution was stirred at room temperature under an atmosphere of argon for 30 minutes. After this time, sodium triacetoxyborohydride (0.102 g, 0.482 mmol) was added. The reaction solution was stirred at room temperature for 1 hour. The reaction was monitored by LC-MS. In some cases, further amine (2 equiv), sodium triacetoxyborohydride (1.1 equiv) and 4 Å molecular sieves (0.050 g) were added and stirring for a maximum of 17 hours was required to progress the reaction further to completion. The reaction mixture was diluted with DCM (20 ml) and washed with sodium hydroxide (5 ml, 2M aq. soln). The organics were extracted using DCM (×2). The combined organics were dried over magnesium sulfate, filtered and concentrated under reduced pressure. The residue was chromatographed [SiO₂, EtOAc:Hexane 75-1% methanolic ammonia in EtOAc]. The resulting residue was stirred in hydrochloric acid in diethyl ether (1 ml, 1 M) for 30 minutes and then concentrated under reduced pressure to give a white solid, N-(1-{[5-chloro-2-(1-methylethyl)-1-benzofuran-7-yl]methyl}-5-methyl-1H-pyrazol-3-yl)-4-[(ethylamino)methyl]benzamide (0.122 g, 61%)

LC/MS Rt=2.64 min, [MH⁺] 465, 467

The following compound was prepared using the above procedure:

| Example | Structure | Name | LC/MS Data |
|---|---|---|---|
| E67 | 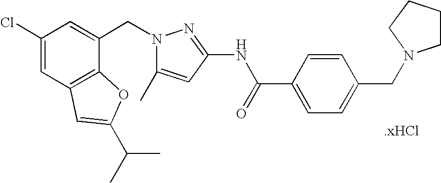 | N-(1-{[5-Chloro-2-(1-methylethyl)-1-benzofuran-7-yl]methyl}-5-methyl-1H-pyrazol-3-yl)-4-{[ethyl(methyl)amino]methyl}benzamidehydrochloride | Rt = 2.63min, [MH⁺]491, 493 |

### Example 68

### N-(1-{[5-Cyano-2-(1-methylethyl)-1-benzofuran-7-yl]methyl}-5-methyl-1H-pyrazol-3-yl)-2-methylpropanamide (E68)

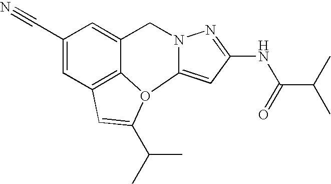

Prepared in a similar manner to N-(1-{[5-chloro-2-(4-cyanophenyl)-1-benzofuran-7-yl]methyl}-5-methyl-1H-pyrazol-3-yl)-2-methylpropanamide using 7-[(3-amino-5-methyl-1H-pyrazol-1-yl)methyl]-2-(1-methylethyl)-1-benzofuran-5-carbonitrile.

LC/MS Rt=3.12 min. Molecular ion observed [MH+] 365, consistent with molecular formula C₂₁H₂₄N₄O₂

### Example 69

### (2R)—N-(1-{[5-Cyano-2-(1-methylethyl)-1-benzofuran-7-yl]methyl}-5-methyl-1H-pyrazol-3-yl)tetrahydro-2-furancarboxamide (E69)

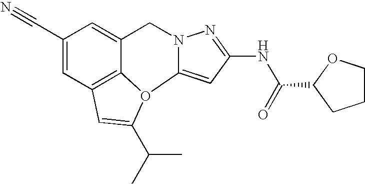

Prepared in a similar manner to (2R)—N-(1-{[5-chloro-2-(4-cyanophenyl)-1-benzofuran-7-yl]methyl}-5-methyl-1H-pyrazol-3-yl)tetrahydro-2-furancarboxamide using 7-[(3-amino-5-methyl-1H-pyrazol-1-yl)methyl]-2-(1-methylethyl)-1-benzofuran-5-carbonitrile.

LC/MS Rt=3.08 min. Molecular ion observed [MH+] 393, consistent with molecular formula C₂₂H₂₄N₄O₃

### Example 70

### N-(1-{[5-Cyano-2-(1-methylethyl)-1-benzofuran-7-yl]methyl}-5-methyl-1H-pyrazol-3-yl)tetrahydro-2H-pyran-4-carboxamide (E70)

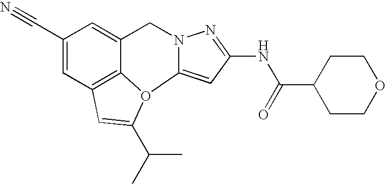

Prepared in a similar manner to N-(1-{[5-chloro-2-(4-cyanophenyl)-1-benzofuran-7-yl]methyl}-5-methyl-1H-pyrazol-3-yl)tetrahydro-2H-pyran-4-carboxamide using 7-[(3-amino-5-methyl-1H-pyrazol-1-yl)methyl]-2-(1-methylethyl)-1-benzofuran-5-carbonitrile except that the title compound was further purified by MDAP.

LC/MS Rt=2.81 min. Molecular ion observed [MH+] 407, consistent with molecular formula C₂₃H₂₆N₄O₃

### Example 71

### 1-{[5-Bromo-2-(1-methylethyl)-1-benzofuran-7-yl]methyl}-N-[4-hydroxymethyl)phenyl]-5-methyl-1H-pyrazole-3-carboxamide (E71)

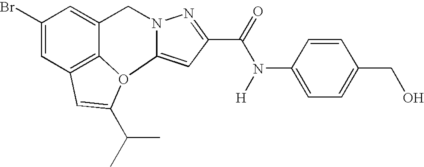

To a solution of 1-{[5-bromo-2-(1-methylethyl)-1-benzofuran-7-yl]methyl}-5-methyl-1H-pyrazole-3-carboxylic acid (755 mg, 2.0 mmol) in dry DMF (10 ml) was added HOBt (368 mg, 2.4 mmol), EDAC (459 mg, 2.4 mmol) and 4-aminobenzyl alcohol (296 mg, 2.4 mmol) and the solution stirred at room temperature under argon for 3.5 hours. EtOAc (60 ml) was added and the mixture extracted with water (60 ml), saturated NaHCO₃ (20 ml) and brine (2×15 ml), dried over magnesium sulphate and evaporated down under reduced pressure. The residue was recrystallised from ethanol to give the title compound as a pale buff solid (766 mg, 79.4%).

LC/MS [M+1]: 482, 484

### Example 72

### 5-{[5-Chloro-2-(1-methylethyl)-1-benzofuran-7-yl]methyl}-N-[4-(hydroxymethyl)phenyl]-2-furancarboxamide (E72)

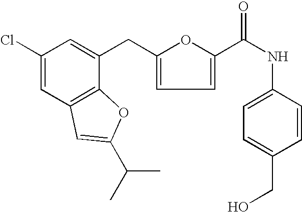

A mixture of 5-{[5-chloro-2-(1-methylethyl)-1-benzofuran-7-yl]methyl}-2-furancarboxylic acid (478 mg, 1.5 mmol), 4-aminobenzyl alcohol (369 mg, 3 mmol), hydroxybenzotriazole (252 mg, 1.65 mmol) and EDAC (383 mg, 2 mmol) in DMF (6 ml) was stirred at room temperature for 3 hours. The resulting solution was diluted with water/ether (50 ml of each) and the organic phase washed with saturated sodium bicarbonate (70 ml), 2M hydrochloric acid (30 ml) and water (3×20 ml) then dried (magnesium sulphate) and evaporated. The resulting white foam was dissolved in 1:2 ether/hexane (4 ml) and left to crystallise to give the title compound (565 mg).

LC/MS: Rt=3.46 min, [M+H]⁺ 424.1, 426.1

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (PCIN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

### Competition Assay Using [³H]-PGE₂

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10M indomethacin for 5 min. The cells are isolated by centrifugation at 250×g for 5mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20mins and the pellet containing the membrane fraction is washed (optional) three times by suspension and centrifugation at 48,000×g for 20mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

Alternatively a similar assay may be carried out using 3-{2-[5-Bromo-2-(2,4-difluoro-benzyloxy)-phenyl]-5-methyl-pyrrol-1-yl}-6-[³H₃-methoxy]methoxy-benzoic acid instead of [³H]-PGE₂.

For the binding assay using 3-{2-[5-Bromo-2-(2,4-difluoro-benzyloxy)-phenyl]-5-methyl-pyrrol-1-yl}-6-[³H₃-methoxy]methoxy-benzoic acid instead of [³H]-PGE₂ the assay is carried out using a similar procedure to that described above using [³H]-PGE₂ with the following changes:

The cell membranes, competing compounds and 3-{2-[5-Bromo-2-(2,4-difluoro-benzyloxy)-phenyl]-5-methyl-pyrrol-1-yl}-6-[³H₃-methoxy]methoxy-benzoic acid (0.2 nM final assay concentration) are incubated in a final volume of 400 μl for 45 min at 37° C. All reagents are prepared in assay buffer. Reactions are terminated by rapid vacuum filtration over GF/B filters using a Brandell cell harvester. The filters are washed with water at ambient temperature, dried and the radioactivity retained on the filters is measured by liquid scintillation counting in Packard TopCount scintillation counter.

The preparation of 3-{2-[5-Bromo-2-(2,4-difluoro-benzyloxy)-phenyl]-5-methyl-pyrrol-1-yl}-6-methoxy-benzoic acid is described in WO 03/101959 and in Hall et al, Biorg. Med. Chem. Lett., 2007, 17, 916-920. The tritiated version may be prepared via conventional routes, e.g. from 3-{2-[5-bromo-2-(2,4-difluoro-benzyloxy)-phenyl]-5-methyl-pyrrol-1-yl}-6-hydroxy-benzoic acid or 3-{2-[5-bromo-2-(2,4-difluoro-benzyloxy)-phenyl]-5-methyl-pyrrol-1-yl}-6-hydroxy-benzoic acid methyl ester.

### Biological Activity at TP Receptor

To determine if a compound has agonist or antagonist activity at the TP receptor a functional calcium mobilisation assay may be performed. Briefly, the antagonist properties of compounds are assessed by their ability to inhibit the mobilisation of intracellular calcium ([Ca²⁺]ᵢ) in response to activation of TP receptors by the stable TXA₂ mimetic U46619 (9,11-dideoxy-11α,9α-epoxy-methanoprostaglandin F2α; commercially available from e.g Sigma-Aldrich). Increasing concentrations of antagonist reduce the amount of calcium that a given concentration of U46619 can mobilise. The net effect is to displace the U46619 concentration-effect curve. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software. The agonist activity of the compounds are determined by their ability to cause an increase in intracellular mobilisation in the absence of U46619.

The human TP calcium mobilisation assay utilises Chinese hamster ovary-K1 (CHO-K1) cells into which a stable (pCIN; BioTechniques 20 (1996): 102-110) vector containing TP cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 96-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of U46619 are then added to the plate in order to assess the antagonist properties of the compounds.

The data so generated may be analysed by means of a computerised curve-fitting routine. The concentration of compound that elicits a half-maximal inhibition of the calcium mobilisation induced by U46619 (pIC₅₀) may then be estimated, and the percentage activation caused by the compounds directly can be used to determine if there is any agonism present.

### Results

The compounds of Examples 1-72 were tested in the binding assay for the human prostanoid EP₁ receptor. The compounds of Examples 1-17, 19, 33, 34, 60 and 61 were tested in the assay using [³H]PGE₂]. The remaining compounds were tested using the assay using 3-{2-[5-Bromo-2-(2,4-difluorobenzyloxy)-phenyl]-5-methyl-pyrrol-1-yl}-6-[³H₃-methoxy]methoxy-benzoic acid. The results are expressed as pIC₅₀ values. A pIC₅₀ is the negative logarithm₁₀ of the IC₅₀. The results given are averages of a number of experiments. The compounds of examples 1-72 had a pIC₅₀ value ≧6. More particularly, the compounds of examples 1-3, 5-7, 9, 14-19, 21-29, 37, 44, 48-53, 55, 60, 62, 63, 67, 68, 71 and 72 exhibited a pIC₅₀ value ≧7.

The compounds of examples 1-7, 9-22, 24-34, 36-48, 50-61, and 66-72 were tested in the human EP₁ calcium mobilisation assay. The results are expressed as functional pKᵢ values. A functional pKi is the negative logarithm₁₀ of the antagonist dissociation constant as determined in the human EP₁ calcium mobilisation assay. The results given are averages of a number of experiments. All compounds of the Examples tested exhibited a functional pKi value >6, except for the compounds of Examples 33, 36, 48 and 58 which exhibited a functional pKi value of >5.8 and ≦6. More particularly, the compounds of examples 1, 3-7, 9, 11-22, 24-29, 31, 32, 37, 39-45, 50-56, 60, 61, and 68-72 exhibited a functional pKi value of ≧6.5.

The compounds of examples 1-4, 6-47, 49-57, and 61-72 were tested in the human EP₃ calcium mobilisation assay. The results are expressed as functional pKᵢ values. A functional pKi is the negative logarithm₁₀ of the antagonist dissociation constant as determined in the human EP₃ calcium mobilisation assay. The results given are averages of a number of experiments. All compounds of the Examples tested exhibited a functional pKi value of ≦5, except for the compounds of Examples 1, 3, 4, 8, 21, 22, 26, 32, 38, 39, 44, 47, 51, 53, 55, 56, 71, and 72 which exhibited a functional pKᵢ value of >5 and ≦6.3.

The compounds of Examples 1-3, 8, 14-17 and 28 were tested in the human TP calcium mobilisation assay. The results are expressed as functional pKᵢ values. A functional pKi is the negative logarithms of the antagonist dissociation constant as determined in the human TP calcium mobilisation assay. The results given are averages of a number of experiments. Of the compounds tested, the compounds of Examples 1, 3, and 14 exhibited a functional pKᵢ value of >5.5. The compound of Example 1 showed a functional pKᵢ value of >7.5. The compounds of examples 2, 8, 15-17 and 28 exhibited a functional pKᵢ value of <5.5.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 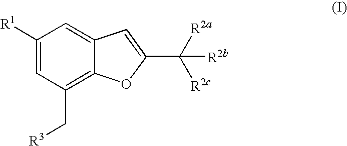
wherein
R¹ is hydrogen, halogen, CF₃, CN, SO₂CH₃ or CH₃;
R^{2a} is hydrogen, C₁₋₄ alkyl, or CF₃;
R^{2b} is C₁₋₄ alkyl or CF₃; and
R^{2c} is hydrogen or CH₃;
or R^{2a} and R^{2b} together with the carbon atom to which they are attached form a C₃₋₆ cycloalkyl group, and R^{2c} is hydrogen;
R³ is: 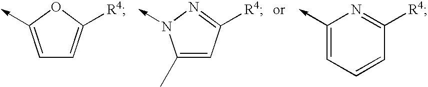
R⁴ is CO₂H, NHCO₂R⁵, CONR^{6a}R^{6b}D, CONHSO₂R⁷, NHCONR⁸R⁹, NHCOR¹⁰, imidazole or tetrazole;
or R⁴ is an imidazole ring fused to give an optionally substituted bicyclic or tricyclic ring system;
R⁵ is C₂₋₆ alkyl, optionally substituted CH₂phenyl or optionally substituted CH₂aliphatic heterocycle;
R^{6a} is hydrogen; and
R^{6b} is hydrogen; NR¹¹R¹²; C₁₋₆alkyl optionally substituted by NR¹¹R¹²; phenyl optionally substituted by halogen, CH₂OH, CH₂NR¹¹R¹², or optionally substituted CH₂aliphatic heterocycle; or optionally substituted (CH₂)ₙaliphatic heterocycle wherein n is 0, 1 or 2;
or R^{6a} and R^{6b} together with the nitrogen atom to which they are attached form an optionally substituted aliphatic heterocycle;
R⁷ is C₁₋₄alkyl, phenyl or heteroaryl;
R⁸ is hydrogen or C₁₋₄alkyl;
R⁹ is C₁₋₄alkyl;
R¹⁰ is C₃₋₆cycloalkyl optionally substituted by OH; C₁₋₆alkyl; or optionally substituted (CH₂)ₘaliphatic heterocycle wherein m is 0, or 1; or
R¹⁰ is 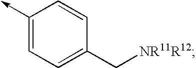
R¹¹ is hydrogen or C₁₋₄alkyl; and
R¹² is hydrogen or C₁₋₄alkyl;
or derivatives thereof;
provided that:
when R^{2c} is CH₃, then R⁴ is CO₂H; and
when R¹ is SO₂CH₃, then R⁴ is CO₂H.

2. A compound according to claim 1 wherein the compound of Formula (I) is a compound of Formula (Ib): 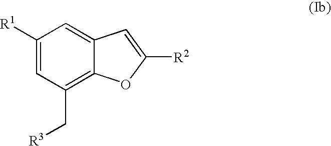
wherein
R¹ is hydrogen, Cl, Br, or CN;
R² is isopropyl, propyl or C₃₋₆ cycloalkyl;
R³ is 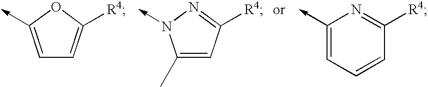
R⁴ is CO₂H, NHCO₂R⁵, CONR^{6a}R^{6b}, CONHSO₂R⁷, NHCONR⁸R⁹, NHCOR¹⁰, imidazole or tetrazole;
or R⁴ is an imidazole ring fused to give an optionally substituted bicyclic or tricyclic ring system;
R⁵ is C₂₋₆ alkyl or optionally substituted CH₂aliphatic heterocycle;
R^{6a} is hydrogen; and
R^{6b} is hydrogen; NR¹¹R¹²; C₁₋₆alkyl optionally substituted by NR¹¹R¹²; phenyl optionally substituted by halogen, CH₂OH, CH₂NR¹¹R¹², or optionally substituted CH₂aliphatic heterocycle; or optionally substituted (CH₂)ₙaliphatic heterocycle wherein n is 0, 1 or 2;
or R^{6a} and R^{6b} together with the nitrogen atom to which they are attached form an optionally substituted aliphatic heterocycle;
R¹⁰ is C₃₋₆cycloalkyl optionally substituted by OH; C₁₋₆alkyl; or optionally substituted (CH₂)ₘaliphatic heterocycle wherein m is 0, or 1; or
R¹⁰ is 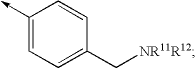
R¹¹ is hydrogen or C₁₋₄alkyl; and
R¹² is hydrogen or C₁₋₄alkyl;
or derivatives thereof.

3. A compound according to claim 1 wherein the compound is selected from the group consisting of
5-{[5-Chloro-2-(1-methylethyl)-1-benzofuran-7-yl]methyl}-2-furancarboxylic acid,
2-(5-{[5-Chloro-2-(1-methylethyl)-1-benzofuran-7-yl]methyl}-2-furanyl)-1H-benzimidazole,
1-{[5-Chloro-2-(1-methylethyl)-1-benzofuran-7-yl]methyl}-5-methyl-1H-pyrazole-3-carboxylic acid,
4-Piperidinylmethyl (1-{[5-chloro-2-(1-methylethyl)-1-benzofuran-7-yl]methyl}-5-methyl-1H-pyrazol-3-yl)carbamate,
2-(1-{[5-Chloro-2-(1-methylethyl)-1-benzofuran-7-yl]methyl}-5-methyl-1H-pyrazol-3-yl)-1H-benzimidazole,
1-{[5-Chloro-2-(1-methylethyl)-1-benzofuran-7-yl]methyl}-5-methyl-N-1-piperidinyl-1H-pyrazole-3-carboxamide,
1-{[5-Chloro-2-(1-methylethyl)-1-benzofuran-7-yl]methyl}-5-methyl-N-4-morpholinyl-1H-pyrazole-3-carboxamide,
5-{[5-Chloro-2-(1-methylethyl)-1-benzofuran-7-yl]methyl}-N-(2,6-difluorophenyl)-2-furancarboxamide,
[2-(5-{[5-Chloro-2-(1-methylethyl)-1-benzofuran-7-yl]methyl}-2-furanyl)-1H-benzimidazol-5-yl]methanol,
{[2-(5-{[5-Chloro-2-(1-methylethyl)-1-benzofuran-7-yl]methyl}-2-furanyl)-1H-benzimidazol-5-yl]methyl}methylamine,
1-[2-(5-{[5-Chloro-2-(1-methylethyl)-1-benzofuran-7-yl]methyl}-2-furanyl)-1H-benzimidazol-5-yl]-N,N-dimethylmethanamine,
2-(5-{[5-Chloro-2-(1-methylethyl)-1-benzofuran-7-yl]methyl}-2-furanyl)-5-(1-pyrrolidinylmethyl)-1H-benzimidazole,
2-(5-{[5-Chloro-2-(1-methylethyl)-1-benzofuran-7-yl]methyl}-2-furanyl)-5-[(4-methyl-1-piperazinyl)methyl]-1H-benzimidazole,
1-{[5-Chloro-2-(1-methylethyl)-1-benzofuran-7-yl]methyl}-5-methyl-N-{4-[(methylamino)methyl]phenyl}-1H-pyrazole-3-carboxamide,
1-{[5-Chloro-2-(1-methylethyl)-1-benzofuran-7-yl]methyl}-N-{4-[(ethylamino)methyl]phenyl}-5-methyl-1H-pyrazole-3-carboxamide,
1-{[5-Chloro-2-(1-methylethyl)-1-benzofuran-7-yl]methyl}-N-{4-[(4-hydroxy-1-Piperidinyl)methyl]phenyl}-5-methyl-1H-pyrazole-3-carboxamide,
1-{[5-Chloro-2-(1-methylethyl)-1-benzofuran-7-yl]methyl}-N-(4-{[(3R)-3-hydroxy-1-pyrrolidinyl]methyl}phenyl)-5-methyl-1H-pyrazole-3-carboxamide,
1-{[5-Bromo-2-(1-methylethyl)-1-benzofuran-7-yl]methyl}-5-methyl-N-4-morpholinyl-1H-pyrazole-3-carboxamide,
1-{[5-Bromo-2-(1-methylethyl)-1-benzofuran-7-yl]methyl}-5-methyl-1H-pyrazole-3-carboxylic acid,
5-Methyl-1-{[2-(1-methylethyl)-1-benzofuran-7-yl]methyl}-1H-pyrazole-3-carboxylic acid
1-{[5-chloro-2-propyl-1-benzofuran-7-yl]methyl}-5-methyl-1H-pyrazole-3-carboxylic acid,
5-methyl-1-{[2-(1-methylethyl)-5-(methylsulfonyl)-1-benzofuran-7-yl]methyl}-1H-pyrazole-3-carboxylic acid,
Sodium 1-[(5-chloro-2-cyclopropyl-1-benzofuran-7-yl]methyl)-5-methyl-1H-pyrazole-3-carboxylate,
5-{[5-Chloro-2-(1-methylethyl)-1-benzofuran-7-yl]methyl}-2-furancarboxylic acid,
6-{[5-Chloro-2-(1-methylethyl)-1-benzofuran-7-yl]methyl}-2-pyridinecarboxylic acid,
1-{[5-Cyano-2-(1-methylethyl)-1-benzofuran-7-yl]methyl}-5-methyl-1H-pyrazole-3-carboxylic acid,
1-[(5-Chloro-2-cyclohexyl-1-benzofuran-7-yl)methyl]-5-methyl-1H-pyrazole-3-carboxylic acid,
1-{[5-Chloro-2-(1′-dimethylethyl)-1-benzofuran-7-yl]methyl}-5-methyl-1H-pyrazole-3-carboxylic acid,
1-[(5-Chloro-2-cyclohexyl-1-benzofuran-7-yl)methyl]-5-methyl-N-4-morpholinyl-1H-pyrazole-3-carboxamide,
1-[(5-Chloro-2-cyclohexyl-1-benzofuran-7-yl)methyl]-5-methyl-N-(tetrahydro-2H-pyran-4-yl)-1H-pyrazole-3-carboxamide,
1-[(5-chloro-2-propyl-1-benzofuran-7-yl)methyl]-5-methyl-N-4-morpholinyl-1H-pyrazole-3-carboxamide,
1-[(5-Chloro-2-cyclopropyl-1-benzofuran-7-yl)methyl]-5-methyl-N-4-morpholinyl-1H-pyrazole-3-carboxamide,
4-({1-[(5-chloro-2-cyclopropyl-1-benzofuran-7-yl)methyl]-5-methyl-1H-pyrazol-3-yl}carbonyl)morpholine,
1-{[5-chloro-2-(1-methylethyl)-1-benzofuran-7-yl]methyl}-5-methyl-N-4-piperidinyl-1H-pyrazole-3-carboxamide,
1-{[5-Chloro-2-(1-methylethyl)-1-benzofuran-7-yl]methyl}-5-methyl-N-(4-piperidinylmethyl)-1H-pyrazole-3-carboxamide,
1-{[5-chloro-2-(1-methylethyl)-1-benzofuran-7-yl]methyl}-N-(1-ethyl-4-piperidinyl)-5-methyl-1H-pyrazole-3-carboxamide,
5-{[5-Chloro-2-(1-methylethyl)-1-benzofuran-7-yl]methyl}-N-(1-methylethyl)-2-furancarboxamide,
5-{[5-Chloro-2-(1-methylethyl)-1-benzofuran-7-yl]methyl}-N′,N′-dimethyl-2-furancarbohydrazide,
6-{[5-Chloro-2-(1-methylethyl)-1-benzofuran-7-yl]methyl}-2-pyridinecarboxamide,
1-{[5-Cyano-2-(1-methylethyl)-1-benzofuran-7-yl]lmethyl}-5-methyl-1H-pyrazole-3-carboxamide,
5-{[5-Chloro-2-(1-methylethyl)-1-benzofuran-7-yl]methyl}-N-4-morpholinyl-2-furancarboxamide,
5-{[5-Chloro-2-(1-methylethyl)-1-benzofuran-7-yl]methyl}-N-1-piperidinyl-2-furancarboxamide,
6-{[5-Chloro-2-(1-methylethyl)-1-benzofuran-7-yl]methyl}-N-4-morpholinyl-2-Pyridinecarboxamide,
1,1-Dimethylethyl 4-({[(6-{[5-chloro-2-(1-methylethyl)-1-benzofuran-7-yl]methyl}-2-pyridinyl)carbonyl]amino}methyl)-1-piperidinecarboxylate,
1-{[5-Cyano-2-(1-methylethyl)-1-benzofuran-7-yl]methyl}-5-methyl-N-4-morpholinyl-1H-pyrazole-3-carboxamide,
1,1-Dimethylethyl 4-({[(1-{[5-cyano-2-(1-methylethyl)-1-benzofuran-7-yl]methyl}-5-methyl-1H-pyrazol-3-yl)carbonyl]amino}methyl)-1-piperidinecarboxylate,
1-{[5-Cyano-2-(1-methylethyl)-1-benzofuran-7-yl]methyl}-5-methyl-N-(4-piperidinylmethyl)-1H-pyrazole-3-carboxamide,
6-{[5-Chloro-2-(1-methylethyl)-1-benzofuran-7-yl]methyl}-N-(4-piperidinylmethyl)-2-pyridinecarboxamide,
5-{[5-Chloro-2-(1-methylethyl)-1-benzofuran-7-yl]methyl}-N-(4-piperidinylmethyl)-2-furancarboxamide,
1-{[5-Bromo-2-(1-methylethyl)-1-benzofuran-7-yl]methyl}-N-{4-[(4-hydroxy-1-piperidinyl)methyl]phenyl}-5-methyl-1H-pyrazole-3-carboxamide,
1-{[5-Bromo-2-(1-methylethyl)-1-benzofuran-7-yl]methyl}-5-methyl-N-{4-[(methylamino)methyl]phenyl}-1H-pyrazole-3-carboxamide,
1-{[5-Bromo-2-(1-methylethyl)-1-benzofuran-7-yl]methyl}-5-methyl-N-[4-(1-pyrrolidinylmethyl)phenyl]-1H-pyrazole-3-carboxamide,
1-{[5-Bromo-2-(1-methylethyl)-1-benzofuran-7-yl]methyl}-5-methyl-N-[4-(4-morpholinylmethyl)phenyl]-1H-pyrazole-3-carboxamide
5-{[5-Chloro-2-(1-methylethyl)-1-benzofuran-7-yl]methyl}-N-[4-(1-pyrrolidinylmethyl)phenyl]-2-furancarboxamide,
5-{[5-Chloro-2-(1-methylethyl)-1-benzofuran-7-yl]methyl}-N-[4-(4-morpholinylmethyl)phenyl]-2-furancarboxamide,
5-{[5-Chloro-2-(1-methylethyl)-1-benzofuran-7-yl]methyl}-N-{4-[(4-hydroxy-1-piperidinyl)methyl]phenyl}-2-furancarboxamide,
5-{[5-Chloro-2-(1-methylethyl)-1-benzofuran-7-yl]methyl}-N-{4-[(ethylamino)methyl]phenyl}-2-furancarboxamide,
1,1-Dimethylethyl (5-{[5-chloro-2-(1-methylethyl)-1-benzofuran-7-yl]methyl}-2-furanyl)carbamate,
5-{[5-Chloro-2-(1-methylethyl)-1-benzofuran-7-yl]methyl}-N-[2-(dimethylamino) ethyl]-2-furancarboxamide,
N-(2-Amino-2-methylpropyl)-5-{[5-chloro-2-(1-methylethyl)-1-benzofuran-7-yl]methyl}-2-furancarboxamide,
1-{[5-chloro-2-(1-methylethyl)-1-benzofuran-7-yl]methyl}-5-methyl-N-(tetrahydro-2H-pyran-4-yl)-1H-pyrazole-3-carboxamide,
1-{[5-Chloro-2-(1-methylethyl)-1-benzofuran-7-yl]methyl}-5-methyl-N-(tetrahydro-2H-pyran-4-ylmethyl)-1H-pyrazole-3-carboxamide,
cis-N-(1-{[5-Chloro-2-(1-methylethyl)-1-benzofuran-7-yl]methyl}-5-methyl-1H-pyrazol-3-yl)-4-hydroxycyclohexanecarboxamide,
trans-N-(1-{[5-chloro-2-(1-methylethyl)-1-benzofuran-7-yl]methyl}-5-methyl-1H-pyrazol-3-yl)-4-hydroxycyclohexanecarboxamide,
1-{[5-Chloro-2-(1-methylethyl)-1-benzofuran-7-yl]methyl}-5-methyl-N-[trans-4-(methyloxy)cyclohexyl]-1H-pyrazole-3-carboxamide,
1-{[5-Chloro-2-(1-methylethyl)-1-benzofuran-7-yl]methyl}-N-[trans-4-(ethyloxy)cyclohexyl]-5-methyl-1H-pyrazole-3-carboxamide,
N-(1-{[5-chloro-2-(1-methylethyl)-1-benzofuran-7-yl]methyl}-5-methyl-1H-pyrazol-3-yl)-4-[(ethylamino)methyl]benzamide,
N-(1-{[5-Chloro-2-(1-methylethyl)-1-benzofuran-7-yl]methyl}-5-methyl-1H-pyrazol-3-yl)-4-{[ethyl(methyl)amino]methyl}benzamide,
N-(1-{[5-Cyano-2-(1-methylethyl)-1-benzofuran-7-yl]methyl}-5-methyl-1H-pyrazol-3-yl)-2-methylpropanamide,
(2R)—N-(1-{[5-Cyano-2-(1-methylethyl)-1-benzofuran-7-yl]methyl}-5-methyl-1H-pyrazol-3-yl)tetrahydro-2-furancarboxamide,
N-(1-{[5-Cyano-2-(1-methylethyl)-1-benzofuran-7-yl]methyl}-5-methyl-1H-pyrazol-3-yl)tetrahydro-2H-pyran-4-carboxamide,
1-{[5-Bromo-2-(1-methylethyl)-1-benzofuran-7-yl]methyl}-N-[4-hydroxymethyl)phenyl]-5-methyl-1H-pyrazole-3-carboxamide, and
5-{[5-Chloro-2-(1-methylethyl)-1-benzofuran-7-yl]methyl}-N-[4-(hydroxymethyl)phenyl]-2-furancarboxamide, and pharmaceutically acceptable derivatives thereof.

4. A pharmaceutical composition comprising a compound according to claim 1 or a pharmaceutically acceptable derivative thereof together with a pharmaceutical carrier and/or excipient.

5. (canceled)

6. (canceled)

7. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

8. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

9. A method of treating a human or animal subject suffering from inflammatory pain, neuropathic pain or visceral pain which method comprises administering to said subject an effective amount of a compound according to claim 1 or a pharmaceutically acceptable derivative thereof.

10. (canceled)

11. (canceled)

12. (canceled)
